SEQUENCE LISTING (85 sequences)

SEQ ID NO: 1 - RNA, 22 nt, Mus <mouse, genus>
  microRNA (up)
aaugcacccgggcaaggauuug

SEQ ID NO: 2 - RNA, 22 nt, Mus <mouse, genus>
  microRNA (up)
uccgucucaguuacuuuauagc

SEQ ID NO: 3 - RNA, 21 nt, Mus <mouse, genus>
  microRNA (up)
acuggacuuggagucagaagg

SEQ ID NO: 4 - RNA, 22 nt, Mus <mouse, genus>
  microRNA (up)
acccgucccguucguccccgga

SEQ ID NO: 5 - RNA, 23 nt, Mus <mouse, genus>
  microRNA (up)
ucucacacagaaaucgcacccgu

SEQ ID NO: 6 - RNA, 22 nt, Mus <mouse, genus>
  microRNA (up)
ucagugcacuacagaacuuugu

SEQ ID NO: 7 - RNA, 22 nt, Mus <mouse, genus>
  microRNA (up)
agaucgaccguguuauauucgc

SEQ ID NO: 8 - RNA, 21 nt, Mus <mouse, genus>
  microRNA (up)
aauauaacacagauggccugu

SEQ ID NO: 9 - RNA, 21 nt, Mus <mouse, genus>
  microRNA (up)
ugucuugcaggccgucaugca

SEQ ID NO: 10 - RNA, 22 nt, Mus <mouse, genus>
  microRNA (up)
ucagugcaucacagaacuuugu

SEQ ID NO: 11 - RNA, 22 nt, Mus <mouse, genus>
  microRNA (up)
uaacagucuccagucacggcca

SEQ ID NO: 12 - RNA, 22 nt, Mus <mouse, genus>
  microRNA (up)
uauggcacugguagaauucacu

SEQ ID NO: 13 - RNA, 21 nt, Mus <mouse, genus>
  mciroRNA (up)
uaguagaccguauagcguacg

SEQ ID NO: 14 - RNA, 22 nt, Mus <mouse, genus>
  microRNA (up)
ucggauccgucugagcuuggcu

SEQ ID NO: 15 - RNA, 23 nt, Mus <mouse, genus>
  microRNA (up)
accuuggcucuagacugcuuacu

SEQ ID NO: 16 - RNA, 22 nt, Mus <mouse, genus>
  microRNA (up)
ugagaacugaauuccauaggcu

SEQ ID NO: 17 - RNA, 23 nt, Mus <mouse, genus>
  mciroRNA (down)
aacauucaacgcugucggugagu

SEQ ID NO: 18 - RNA, 23 nt, Mus <mouse, genus>
  microRNA (down)
uacccuguagauccgaauuugug

SEQ ID NO: 19 - RNA, 23 nt, Mus <mouse, genus>
  microRNA (down)
aacauucauugcugucggugggu

SEQ ID NO: 20 - RNA, 21 nt, Mus <mouse, genus>
  microRNA (down)
aauggcgccacuaggguugug

SEQ ID NO: 21 - RNA, 22 nt, Mus <mouse, genus>
  microRNA (down)
ugagaacugaauuccauggguu

SEQ ID NO: 22 - RNA, 21 nt, Mus <mouse, genus>
  microRNA (down)
cuagacugaggcuccuugagg

SEQ ID NO: 23 - RNA, 21 nt, Mus <mouse, genus>
  microRNA (down)
uagcagcacagaaauauuggc

SEQ ID NO: 24 - RNA, 22 nt, Mus <mouse, genus>
  microRNA (down)
gaguauuguuuccacugccugg

SEQ ID NO: 25 - RNA, 22 nt, Mus <mouse, genus>
  microRNA (down)
gugaaauguuuaggaccacuag

SEQ ID NO: 26 - RNA, 21 nt, Mus <mouse, genus>
  microRNA (down)
ccguccugagguuguugagcu

SEQ ID NO: 27 - RNA, 22 nt, Mus <mouse, genus>
  microRNA (down)
acaggcugucugaucccacggu

SEQ ID NO: 28 - RNA, 22 nt, Mus <mouse, genus>
  microRNA (down)
guaaacauccgacugaaagcuc

SEQ ID NO: 29 - protein, 5 aa, Artificial Sequence
  peptide specific for lung - Seq ID No. 1 WO 2015/018860
GHGYF

SEQ ID NO: 30 - protein, 7 aa, Artificial Sequence
  lung specifice peptide - Seq ID No. 2 WO 2015/018860
ESGHGYF

SEQ ID NO: 31 - protein, 744 aa, Artificial Sequence
  Seq ID No. 9 WO 2015/018860
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGQRGESGHGYFAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 32 - DNA, 1774 bp, Artificial Sequence
  CMV-mir181a-scAAV Double stranded AAV vector genome for simultaneous expression of a cDNA (eGFP) and a miRNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgaaacattcaacgctgtcggtgagttagtgaagccacagatgtaaccatcgaccgttgattgtaccgtgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 33 - DNA, 1719 bp, Artificial Sequence
  CMV-mir181a-mir181b-mir10a-scAAV Double stranded AAV vector genome for simultaneous expression of three miRNAs
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaaggcgcgcctcgacttcttaacccaacagaaggctcgagaaggtatattgctgttgacagtgagcgaaacattcaacgctgtcggtgagttagtgaagccacagatgtaaccatcgaccgttgattgtaccgtgcctactgcctcggacttcaaggggctagaattcgatcgacttcttaacccaacagaaggctcgagaaggtatattgctgttgacagtgagcgaaacattcattgctgtcggtgggtttagtgaagccacagatgtactcactgatcaatgaatgcaaagtgcctactgcctcggacttcaaggggctagaattcgatcgacttcttaacccaacagaaggctcgagaaggtatattgctgttgacagtgagcgataccctgtagatccgaatttgtgtagtgaagccacagatgtacaaattcgtatctaggggaatagtgcctactgcctcggacttcaaggggctagaattcgagaacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggagaacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 34 - RNA, 22 nt, Homo sapiens
  microRNA (up)
aaugcacccgggcaaggauucu

SEQ ID NO: 35 - RNA, 22 nt, Homo sapiens
  miRNA (up)
acuggacuuggagucagaaggc

SEQ ID NO: 36 - RNA, 21 nt, Homo sapiens
  miRNA (up)
uaacagucuccagucacggcc

SEQ ID NO: 37 - RNA, 21 nt, Homo sapiens
  miRNA (down)
cuagacugaagcuccuugagg

SEQ ID NO: 38 - RNA, 21 nt, Homo sapiens
  miRNA (down)
uagcagcacagaaauauuggc

SEQ ID NO: 39 - RNA, 23 nt, Homo sapiens
  miRNA (down)
gggguauuguuuccgcugccagg

SEQ ID NO: 40 - DNA, 159 bp, Artificial Sequence
  >mir-10a-5p 23 nt, miR-E backbone, Passenger position
tcgacttcttaacccaacagaaggctcgagaaggtatattgctgttgacagtgagcgtaccctgtagatccgaatttgtgtagtgaagccacagatgtacacaaattcggatctacagggtctgcctactgcctcggacttcaaggggctagaattcga

SEQ ID NO: 41 - DNA, 159 bp, Artificial Sequence
  >mir-10a-5p 23 nt, miR-E backbone, Guide position
tcgacttcttaacccaacagaaggctcgagaaggtatattgctgttgacagtgagcgaacaaattcggatctacagggtatagtgaagccacagatgtataccctgtagatccgaatttgtgtgcctactgcctcggacttcaaggggctagaattcga

SEQ ID NO: 42 - DNA, 157 bp, Artificial Sequence
  >mir-10a-5p 22 nt, miR-E backbone, Passenger position
tcgacttcttaacccaacagaaggctcgagaaggtatattgctgttgacagtgagcgtaccctgtagatccgaatttgttagtgaagccacagatgtaacaaattcggatctacagggtctgcctactgcctcggacttcaaggggctagaattcga

SEQ ID NO: 43 - DNA, 157 bp, Artificial Sequence
  >mir-10a-5p 22 nt, miR-E backbone, Guide position
tcgacttcttaacccaacagaaggctcgagaaggtatattgctgttgacagtgagcgccaaattcggatctacagggtatagtgaagccacagatgtataccctgtagatccgaatttgttgcctactgcctcggacttcaaggggctagaattcga

SEQ ID NO: 44 - DNA, 184 bp, Artificial Sequence
  >mir-10a-5p, natural pre-miRNA in miR-E backbone, Human (hsa-mir-10a MI0000266)
tcgacttcttaacccaacagaaggctcgagaaggtatattgctgttggatctgtctgtcttctgtatataccctgtagatccgaatttgtgtaaggaattttgtggtcacaaattcgtatctaggggaatatgtagttgacataaacactccgctctctcggacttcaaggggctagaattcga

SEQ ID NO: 45 - DNA, 184 bp, Artificial Sequence
  >mir-10a-5p, natural pre-miRNA in miR-E backbone, Mouse (mmu-mir-10a MI0000685)
tcgacttcttaacccaacagaaggctcgagaaggtatattgctgttggacctgtctgtcttctgtatataccctgtagatccgaatttgtgtaaggaattttgtggtcacaaattcgtatctaggggaatatgtagttgacataaacactccgctcactcggacttcaaggggctagaattcga

SEQ ID NO: 46 - DNA, 159 bp, Artificial Sequence
  >mir-181a-5p 23 nt, miR-E backbone, Passenger position
tcgacttcttaacccaacagaaggctcgagaaggtatattgctgttgacagtgagcgaacattcaacgctgtcggtgagttagtgaagccacagatgtaactcaccgacagcgttgaatgtgtgcctactgcctcggacttcaaggggctagaattcga

SEQ ID NO: 47 - DNA, 159 bp, Artificial Sequence
  >mir-181a-5p 23 nt, miR-E backbone, Guide position
tcgacttcttaacccaacagaaggctcgagaaggtatattgctgttgacagtgagcgcctcaccgacagcgttgaatgtttagtgaagccacagatgtaaacattcaacgctgtcggtgagttgcctactgcctcggacttcaaggggctagaattcga

SEQ ID NO: 48 - DNA, 157 bp, Artificial Sequence
  >mir-181a-5p 22 nt, miR-E backbone, Passenger position
tcgacttcttaacccaacagaaggctcgagaaggtatattgctgttgacagtgagcgaacattcaacgctgtcggtgagtagtgaagccacagatgtactcaccgacagcgttgaatgtgtgcctactgcctcggacttcaaggggctagaattcga

SEQ ID NO: 49 - DNA, 157 bp, Artificial Sequence
  >mir-181a-5p 22 nt, miR-E backbone, Guide position
tcgacttcttaacccaacagaaggctcgagaaggtatattgctgttgacagtgagcgatcaccgacagcgttgaatgtttagtgaagccacagatgtaaacattcaacgctgtcggtgagtgcctactgcctcggacttcaaggggctagaattcga

SEQ ID NO: 50 - DNA, 184 bp, Artificial Sequence
  >mir-181a-5p, natural pre-miRNA in miR-E backbone, Human (hsa-mir-181a-1 MI0000289)
tcgacttcttaacccaacagaaggctcgagaaggtatattgctgttgtgagttttgaggttgcttcagtgaacattcaacgctgtcggtgagtttggaattaaaatcaaaaccatcgaccgttgattgtaccctatggctaaccatcatctactccactcggacttcaaggggctagaattcga

SEQ ID NO: 51 - DNA, 161 bp, Artificial Sequence
  >mir-181a-5p, natural pre-miRNA in miR-E backbone, Mouse (mmu-mir-181a-1 MI0000697)
tcgacttcttaacccaacagaaggctcgagaaggtatattgctgttgggttgcttcagtgaacattcaacgctgtcggtgagtttggaattcaaataaaaaccatcgaccgttgattgtaccctatagctaaccctcggacttcaaggggctagaattcga

SEQ ID NO: 52 - DNA, 159 bp, Artificial Sequence
  >mir-181b-5p 23 nt, miR-E backbone, Passenger position
tcgacttcttaacccaacagaaggctcgagaaggtatattgctgttgacagtgagcgaacattcattgctgtcggtgggttagtgaagccacagatgtaacccaccgacagcaatgaatgtgtgcctactgcctcggacttcaaggggctagaattcga

SEQ ID NO: 53 - DNA, 159 bp, Artificial Sequence
  >mir-181b-5p 23 nt, miR-E backbone, Guide position
tcgacttcttaacccaacagaaggctcgagaaggtatattgctgttgacagtgagcgccccaccgacagcaatgaatgtttagtgaagccacagatgtaaacattcattgctgtcggtgggttgcctactgcctcggacttcaaggggctagaattcga

SEQ ID NO: 54 - DNA, 157 bp, Artificial Sequence
  >mir-181b-5p 22 nt, miR-E backbone, Passenger position
tcgacttcttaacccaacagaaggctcgagaaggtatattgctgttgacagtgagcgaacattcattgctgtcggtgggtagtgaagccacagatgtacccaccgacagcaatgaatgtgtgcctactgcctcggacttcaaggggctagaattcga

SEQ ID NO: 55 - DNA, 157 bp, Artificial Sequence
  >mir-181b-5p 22 nt, miR-E backbone, Guide position
tcgacttcttaacccaacagaaggctcgagaaggtatattgctgttgacagtgagcgaccaccgacagcaatgaatgtttagtgaagccacagatgtaaacattcattgctgtcggtgggtgcctactgcctcggacttcaaggggctagaattcga

SEQ ID NO: 56 - DNA, 184 bp, Artificial Sequence
  >mir-181b-5p, natural pre-miRNA in miR-E backbone, Human (hsa-mir-181b-1 MI0000270)
tcgacttcttaacccaacagaaggctcgagaaggtatattgctgttgcctgtgcagagattattttttaaaaggtcacaatcaacattcattgctgtcggtgggttgaactgtgtggacaagctcactgaacaatgaatgcaactgtggccccgcttctcggacttcaaggggctagaattcga

SEQ ID NO: 57 - DNA, 154 bp, Artificial Sequence
  >mir-181b-5p, natural pre-miRNA in miR-E backbone, Mouse (mmu-mir-181b-1 MI0000723)
tcgacttcttaacccaacagaaggctcgagaaggtatattgctgttgaggtcacaatcaacattcattgctgtcggtgggttgaactgtgtagaaaagctcactgaacaatgaatgcaactgtggccctcggacttcaaggggctagaattcga

SEQ ID NO: 58 - DNA, 159 bp, Artificial Sequence
  >mir-212-5p 23 nt, miR-E backbone, Passenger position
tcgacttcttaacccaacagaaggctcgagaaggtatattgctgttgacagtgagcgaccttggctctagactgcttacttagtgaagccacagatgtaagtaagcagtctagagccaaggctgcctactgcctcggacttcaaggggctagaattcga

SEQ ID NO: 59 - DNA, 159 bp, Artificial Sequence
  >mir-212-5p 23 nt, miR-E backbone, Guide position
tcgacttcttaacccaacagaaggctcgagaaggtatattgctgttgacagtgagcgcgtaagcagtctagagccaaggttagtgaagccacagatgtaaccttggctctagactgcttacttgcctactgcctcggacttcaaggggctagaattcga

SEQ ID NO: 60 - DNA, 157 bp, Artificial Sequence
  >mir-212-5p 22 nt, miR-E backbone, Passenger position
tcgacttcttaacccaacagaaggctcgagaaggtatattgctgttgacagtgagcgaccttggctctagactgcttactagtgaagccacagatgtagtaagcagtctagagccaaggctgcctactgcctcggacttcaaggggctagaattcga

SEQ ID NO: 61 - DNA, 157 bp, Artificial Sequence
  >mir-212-5p 22 nt, miR-E backbone, Guide position
tcgacttcttaacccaacagaaggctcgagaaggtatattgctgttgacagtgagcgataagcagtctagagccaaggttagtgaagccacagatgtaaccttggctctagactgcttactgcctactgcctcggacttcaaggggctagaattcga

SEQ ID NO: 62 - DNA, 184 bp, Artificial Sequence
  >mir-212-5p, natural pre-miRNA in miR-E backbone, Human (hsa-mir-212 MI0000288)
tcgacttcttaacccaacagaaggctcgagaaggtatattgctgttgcggggcaccccgcccggacagcgcgccggcaccttggctctagactgcttactgcccgggccgccctcagtaacagtctccagtcacggccaccgacgcctggccccgccctcggacttcaaggggctagaattcga

SEQ ID NO: 63 - DNA, 165 bp, Artificial Sequence
  >mir-212-5p, natural pre-miRNA in miR-E backbone, Mouse (mmu-mir-212 MI0000696)
tcgacttcttaacccaacagaaggctcgagaaggtatattgctgttggggcagcgcgccggcaccttggctctagactgcttactgcccgggccgccttcagtaacagtctccagtcacggccaccgacgcctggcccctcggacttcaaggggctagaattcga

SEQ ID NO: 64 - DNA, 1773 bp, Artificial Sequence
  >scAAV-CMV-eGFP-mir181b-5p(23 nt in miR-E backbone)-SV40pA, Passenger position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgaacattcattgctgtcggtgggttagtgaagccacagatgtaacccaccgacagcaatgaatgtgtgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 65 - DNA, 1773 bp, Artificial Sequence
  >scAAV-CMV-eGFP-mir181b-5p(23 nt in miR-E backbone)-SV40pA, Guide position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgccccaccgacagcaatgaatgtttagtgaagccacagatgtaaacattcattgctgtcggtgggttgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 66 - DNA, 1771 bp, Artificial Sequence
  >scAAV-CMV-eGFP-mir181b-5p(22 nt in miR-E backbone)-SV40pA, Passenger position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgaacattcattgctgtcggtgggtagtgaagccacagatgtacccaccgacagcaatgaatgtgtgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 67 - DNA, 1771 bp, Artificial Sequence
  >scAAV-CMV-eGFP-mir181b-5p(22 nt in miR-E backbone)-SV40pA, Guide position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgaccaccgacagcaatgaatgtttagtgaagccacagatgtaaacattcattgctgtcggtgggtgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 68 - DNA, 1798 bp, Artificial Sequence
  >scAAV-CMV-eGFP-mir181b-5p(natural pre-miRNA, human)-SV40pA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgcctgtgcagagattattttttaaaaggtcacaatcaacattcattgctgtcggtgggttgaactgtgtggacaagctcactgaacaatgaatgcaactgtggccccgcttctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 69 - DNA, 1768 bp, Artificial Sequence
  >scAAV-CMV-eGFP-mir181b-5p(natural pre-miRNA, mouse)-SV40pA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgaggtcacaatcaacattcattgctgtcggtgggttgaactgtgtagaaaagctcactgaacaatgaatgcaactgtggccctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 70 - DNA, 2091 bp, Artificial Sequence
  >scAAV-CMV-eGFP-mir-181a-mir181b-mir10a(all 23 nt in miR-E backbone), Passenger position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgaacattcaacgctgtcggtgagttagtgaagccacagatgtaactcaccgacagcgttgaatgtgtgcctactgcctcggacttcaaggggctagaattcgatcgacttcttaacccaacagaaggctcgagaaggtatattgctgttgacagtgagcgaacattcattgctgtcggtgggttagtgaagccacagatgtaacccaccgacagcaatgaatgtgtgcctactgcctcggacttcaaggggctagaattcgatcgacttcttaacccaacagaaggctcgagaaggtatattgctgttgacagtgagcgtaccctgtagatccgaatttgtgtagtgaagccacagatgtacacaaattcggatctacagggtctgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 71 - DNA, 2091 bp, Artificial Sequence
  >scAAV-CMV-eGFP-mir-181a-mir181b-mir10a(all 23 nt in miR-E backbone), Guide position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgcctcaccgacagcgttgaatgtttagtgaagccacagatgtaaacattcaacgctgtcggtgagttgcctactgcctcggacttcaaggggctagaattcgatcgacttcttaacccaacagaaggctcgagaaggtatattgctgttgacagtgagcgccccaccgacagcaatgaatgtttagtgaagccacagatgtaaacattcattgctgtcggtgggttgcctactgcctcggacttcaaggggctagaattcgatcgacttcttaacccaacagaaggctcgagaaggtatattgctgttgacagtgagcgaacaaattcggatctacagggtatagtgaagccacagatgtataccctgtagatccgaatttgtgtgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 72 - DNA, 2085 bp, Artificial Sequence
  >scAAV-CMV-eGFP-mir-181a-mir181b-mir10a(all 22 nt in miR-E backbone), Passenger position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgaacattcaacgctgtcggtgagtagtgaagccacagatgtactcaccgacagcgttgaatgtgtgcctactgcctcggacttcaaggggctagaattcgatcgacttcttaacccaacagaaggctcgagaaggtatattgctgttgacagtgagcgaacattcattgctgtcggtgggtagtgaagccacagatgtacccaccgacagcaatgaatgtgtgcctactgcctcggacttcaaggggctagaattcgatcgacttcttaacccaacagaaggctcgagaaggtatattgctgttgacagtgagcgtaccctgtagatccgaatttgttagtgaagccacagatgtaacaaattcggatctacagggtctgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 73 - DNA, 2085 bp, Artificial Sequence
  >scAAV-CMV-eGFP-mir-181a-mir181b-mir10a(all 22 nt in miR-E backbone), Guide position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgatcaccgacagcgttgaatgtttagtgaagccacagatgtaaacattcaacgctgtcggtgagtgcctactgcctcggacttcaaggggctagaattcgatcgacttcttaacccaacagaaggctcgagaaggtatattgctgttgacagtgagcgaccaccgacagcaatgaatgtttagtgaagccacagatgtaaacattcattgctgtcggtgggtgcctactgcctcggacttcaaggggctagaattcgatcgacttcttaacccaacagaaggctcgagaaggtatattgctgttgacagtgagcgccaaattcggatctacagggtatagtgaagccacagatgtataccctgtagatccgaatttgttgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 74 - DNA, 2091 bp, Artificial Sequence
  >scAAV-CMV-eGFP-mir-212-5p-mir181b-mir10a(all 23 nt in miR-E backbone), Passenger position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgaccttggctctagactgcttacttagtgaagccacagatgtaagtaagcagtctagagccaaggctgcctactgcctcggacttcaaggggctagaattcgatcgacttcttaacccaacagaaggctcgagaaggtatattgctgttgacagtgagcgaacattcattgctgtcggtgggttagtgaagccacagatgtaacccaccgacagcaatgaatgtgtgcctactgcctcggacttcaaggggctagaattcgatcgacttcttaacccaacagaaggctcgagaaggtatattgctgttgacagtgagcgtaccctgtagatccgaatttgtgtagtgaagccacagatgtacacaaattcggatctacagggtctgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 75 - DNA, 2091 bp, Artificial Sequence
  >scAAV-CMV-eGFP-mir-212-5p-mir181b-mir10a(all 23 nt in miR-E backbone), Guide position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgcgtaagcagtctagagccaaggttagtgaagccacagatgtaaccttggctctagactgcttacttgcctactgcctcggacttcaaggggctagaattcgatcgacttcttaacccaacagaaggctcgagaaggtatattgctgttgacagtgagcgccccaccgacagcaatgaatgtttagtgaagccacagatgtaaacattcattgctgtcggtgggttgcctactgcctcggacttcaaggggctagaattcgatcgacttcttaacccaacagaaggctcgagaaggtatattgctgttgacagtgagcgaacaaattcggatctacagggtatagtgaagccacagatgtataccctgtagatccgaatttgtgtgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 76 - DNA, 2085 bp, Artificial Sequence
  >scAAV-CMV-eGFP-mir-212-5p-mir181b-mir10a(all 22 nt in miR-E backbone), Passenger position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgaccttggctctagactgcttactagtgaagccacagatgtagtaagcagtctagagccaaggctgcctactgcctcggacttcaaggggctagaattcgatcgacttcttaacccaacagaaggctcgagaaggtatattgctgttgacagtgagcgaacattcattgctgtcggtgggtagtgaagccacagatgtacccaccgacagcaatgaatgtgtgcctactgcctcggacttcaaggggctagaattcgatcgacttcttaacccaacagaaggctcgagaaggtatattgctgttgacagtgagcgtaccctgtagatccgaatttgttagtgaagccacagatgtaacaaattcggatctacagggtctgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 77 - DNA, 2085 bp, Artificial Sequence
  >scAAV-CMV-eGFP-mir-212-5p-mir181b-mir10a(all 22 nt in miR-E backbone), Guide position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgctaagcagtctagagccaaggttagtgaagccacagatgtaaccttggctctagactgcttactgcctactgcctcggacttcaaggggctagaattcgatcgacttcttaacccaacagaaggctcgagaaggtatattgctgttgacagtgagcgaccaccgacagcaatgaatgtttagtgaagccacagatgtaaacattcattgctgtcggtgggtgcctactgcctcggacttcaaggggctagaattcgatcgacttcttaacccaacagaaggctcgagaaggtatattgctgttgacagtgagcgccaaattcggatctacagggtatagtgaagccacagatgtataccctgtagatccgaatttgttgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 78 - DNA, 2166 bp, Artificial Sequence
  >scAAV-CMV-eGFP-mir-181a-mir181b-mir10a(natural pre-miRNAs in miR-E backbone), Human
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgtgagttttgaggttgcttcagtgaacattcaacgctgtcggtgagtttggaattaaaatcaaaaccatcgaccgttgattgtaccctatggctaaccatcatctactccactcggacttcaaggggctagaattcgatcgacttcttaacccaacagaaggctcgagaaggtatattgctgttgcctgtgcagagattattttttaaaaggtcacaatcaacattcattgctgtcggtgggttgaactgtgtggacaagctcactgaacaatgaatgcaactgtggccccgcttctcggacttcaaggggctagaattcgatcgacttcttaacccaacagaaggctcgagaaggtatattgctgttggatctgtctgtcttctgtatataccctgtagatccgaatttgtgtaaggaattttgtggtcacaaattcgtatctaggggaatatgtagttgacataaacactccgctctctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 79 - DNA, 2113 bp, Artificial Sequence
  >scAAV-CMV-eGFP-mir-181a-mir181b-mir10a(natural pre-miRNAs in miR-E backbone), Mouse
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgggttgcttcagtgaacattcaacgctgtcggtgagtttggaattcaaataaaaaccatcgaccgttgattgtaccctatagctaaccctcggacttcaaggggctagaattcgatcgacttcttaacccaacagaaggctcgagaaggtatattgctgttgaggtcacaatcaacattcattgctgtcggtgggttgaactgtgtagaaaagctcactgaacaatgaatgcaactgtggccctcggacttcaaggggctagaattcgatcgacttcttaacccaacagaaggctcgagaaggtatattgctgttggacctgtctgtcttctgtatataccctgtagatccgaatttgtgtaaggaattttgtggtcacaaattcgtatctaggggaatatgtagttgacataaacactccgctcactcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 80 - DNA, 2166 bp, Artificial Sequence
  >scAAV-CMV-eGFP-mir-212-5p-mir181b- mir10a(natural pre-miRNAs in miR-E backbone), Human
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgcggggcaccccgcccggacagcgcgccggcaccttggctctagactgcttactgcccgggccgccctcagtaacagtctccagtcacggccaccgacgcctggccccgccctcggacttcaaggggctagaattcgatcgacttcttaacccaacagaaggctcgagaaggtatattgctgttgcctgtgcagagattattttttaaaaggtcacaatcaacattcattgctgtcggtgggttgaactgtgtggacaagctcactgaacaatgaatgcaactgtggccccgcttctcggacttcaaggggctagaattcgatcgacttcttaacccaacagaaggctcgagaaggtatattgctgttggatctgtctgtcttctgtatataccctgtagatccgaatttgtgtaaggaattttgtggtcacaaattcgtatctaggggaatatgtagttgacataaacactccgctctctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 81 - DNA, 2117 bp, Artificial Sequence
  >scAAV-CMV-eGFP-mir-212-5p-mir181b- mir10a(natural pre-miRNAs in miR-E backbone), Mouse
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttggggcagcgcgccggcaccttggctctagactgcttactgcccgggccgccttcagtaacagtctccagtcacggccaccgacgcctggcccctcggacttcaaggggctagaattcgatcgacttcttaacccaacagaaggctcgagaaggtatattgctgttgaggtcacaatcaacattcattgctgtcggtgggttgaactgtgtagaaaagctcactgaacaatgaatgcaactgtggccctcggacttcaaggggctagaattcgatcgacttcttaacccaacagaaggctcgagaaggtatattgctgttggacctgtctgtcttctgtatataccctgtagatccgaatttgtgtaaggaattttgtggtcacaaattcgtatctaggggaatatgtagttgacataaacactccgctcactcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 82 - protein, 736 aa, Artificial Sequence
  AAV6.2 VP1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 83 - DNA, 157 bp, Artificial Sequence
  mir- Ren713, neutral control, mirE backbone
tcgacttcttaacccaacagaaggctcgagaaggtatattgctgttgacagtgagcgcaggaattataatgcttatctatagtgaagccacagatgtatagataagcattataattcctatgcctactgcctcggacttcaaggggctagaattcga

SEQ ID NO: 84 - DNA, 182 bp, Artificial Sequence
  mir-181a stem-loop, miR-E context
tcgacttcttaacccaacagaaggctcgagaaggtatattgctgtttgagttttgaggttgcttcagtgaacattcaacgctgtcggtgagtttggaattaaaatcaaaaccatcgaccgttgattgtaccctatggctaaccatcatctactccatcggacttcaaggggctagaattcga

SEQ ID NO: 85 - DNA, 182 bp, Artificial Sequence
  mir-212 stem-loop, miR-E context
tcgacttcttaacccaacagaaggctcgagaaggtatattgctgttcggggcaccccgcccggacagcgcgccggcaccttggctctagactgcttactgcccgggccgccctcagtaacagtctccagtcacggccaccgacgcctggccccgcctcggacttcaaggggctagaattcga

(19) United States
(12) Patent Application Publication
(10) Pub. No.: US 2022/0213503 A1
(43) Pub. Date: Jul. 7, 2022

(54) VIRAL VECTORS AND NUCLEIC ACIDS FOR USE IN THE TREATMENT OF PF-ILD AND IPF

(71) Applicant: Boehringer Ingelheim International GmbH, Ingelheim am Rhein (DE)

(72) Inventors: Sebastian Kreuz, Ingelheim am Rhein (DE); Holger Klein, Ingelheim am Rhein (DE); Benjamin Strobel, Ingelheim am Rhein (DE); Stephan Klee, Ingelheim am Rhein (DE); Thorsten Lamla, Ingelheim am Rhein (DE); Marc Kaestle, Ingelheim am Rhein (DE)

(21) Appl. No.: 17/608,328

(22) PCT Filed: Apr. 30, 2020
(86) PCT No.: PCT/EP2020/062174

§ 371 Date: Nov. 2, 2021

Related U.S. Application Data
(63) Continuation of application No. PCT/EP2019/061323, filed on May 2, 2019.

(30) Foreign Application Priority Data
May 2, 2019 (EP) PCT/EP2019/061323

(51) Int. Cl. C12N 15/86; C12N 15/113

(52) U.S. Cl. CPC C12N 15/86; C12N 15/113; C12N 2310/113; C12N 2750/14143; C12N 2320/31; C12N 2320/32; C12N 2330/51; C12N 2310/141

(57) ABSTRACT

Viral vector comprising: a capsid and a packaged nucleic acid, wherein the nucleic acid either augments the miRNA downregulated in a Bleomycin-induced lung fibrosis model or in an AAV-TGFβ1-induced lung fibrosis model, or wherein the nucleic acid inhibits the miRNA up-regulated in a Bleomycin-induced lung fibrosis model or in an AAV-TGFβ1-induced lung fibrosis model.

### BACKGROUND OF THE INVENTION

Progressive fibrosing interstitial lung diseases (PF-ILD), such as idiopathic pulmonary fibrosis (IPF), connective tissue disease (CTD)-associated interstitial lung disease (ILD), systemic sclerosis ILD, rheumatoid arthritis ILD, chronic fibrosing hypersensitivity pneumonitis (HP), idiopathic non-specific interstitial pneumonia (iNSIP), unclassifiable idiopathic interstitial pneumonia (IIP), environmental/occupational lung disease and sarcoidosis, encompass a variety of different clinical settings that include a fibrosing pulmonary phenotype. Idiopathic pulmonary fibrosis (IPF), the most common and severe condition, is a disabling, progressive, and ultimately fatal disease, which is characterized by fibrosis of the lung parenchyma and loss of pulmonary function (Raghu G et al., 2011). The etiology of IPF is still unknown; however various irritants including smoking, occupational hazards, viral and bacterial infections as well as radiotherapy and chemotherapeutic agents (like e.g. Bleomycin) have been described as potential risk factors for the development of IPF. Due to changes in IPF diagnostic criteria over the past years, the prevalence of IPF varies considerably in the literature. According to recent data, the prevalence of IPF ranges from 14.0 to 63.0 cases per 100,000 while the incidence lies between 6.8 and 17.4 new annual cases per 100,000 (Ley B et al., 2013). IPF is usually diagnosed in elderly people with an average age of disease onset of 66 (Hopkins R B et al., 2016). After initial diagnosis IPF progresses rapidly with a mortality rate of approximately 60 percent within 3 to 5 years. In contrast to IPF, only some of the patients with CTD (including e.g. rheumatoid arthritis (RA), Sjögren's syndrome and systemic sclerosis (SSc)) or sarcoidosis display a PF-ILD phenotype, with about 10-20% of RA patients, 9-24% of Sjögren's syndrome, >70% of SSc (Mathai S C and Danoff S K, 2016) and 20-25% of sarcoidosis patients (Spagnolo P et al., 2018) developing pulmonary fibrosis.

There are two main histopathological characteristics observed in PF-ILDs, namely nonspecific interstitial pneumonia (NSIP) and usual interstitial pneumonitis (UIP). The histopathological hallmarks of IPF are UIP and progressive interstitial fibrosis caused by excessive extracellular matrix deposition. UIP is characterized by a heterogeneous appearance with areas of subpleural and paraseptal fibrosis alternating with areas of less affected or normal lung parenchyma. Areas of active fibrosis, so-called fibroblastic foci, are characterized by fibroblast accumulation and excessive collagen deposition. Fibroblastic foci are frequently located between the vascular endothelium and the alveolar epithelium, thereby causing disruption of lung architecture and formation of characteristic “honeycomb”-like structures. Clinical manifestations of IPF are dramatically compromised oxygen diffusion, progressive decline of lung function, cough and severe impairments in quality of life. UIP is also the main histopathological hallmark in RA-ILD and late-stage sarcoidosis; however, other CTDs, such as SSc or Sjögren's, are mainly characterized by non-specific interstitial pneumonia (NSIP).

NSIP is characterized by less spatial heterogeneity, i.e. pathological anomalies are rather uniformly spread across the lung. In the cellular NSIP subtype, histopathology is characterized by inflammatory cells, whereas in the more common fibrotic subtype, additional areas of pronounced fibrosis are evident. However, pathological manifestations can be diverse, thereby complicating correct diagnosis and differentiation from other types of fibrosis, such as UIP/IPF.

Due to the unknown disease cause of IPF, the knowledge regarding pathological mechanisms on the cellular and molecular level is still limited. However, recent advances in translational research using experimental disease models (in vitro and in vivo) for functional studies as well as tissue samples from IPF patients for genomics/proteomics analyses enabled valuable insights into key disease mechanisms. According to our current understanding, IPF is initiated through repeated alveolar epithelial cell (AEC) micro-injuries, which finally result in an uncontrolled and persistent wound healing response. In more detail, AEC damage induces an aberrant activation of neighboring epithelial cells, thereby leading to the recruitment of immune cells and stem or progenitor cells to the sites of injury. By secreting various cytokines, chemokines and growth factors, infiltrating cells produce a pro-inflammatory environment, which finally results in the expansion and activation of fibroblasts. Under physiological conditions these so-called myofibroblasts produce extracellular matrix (ECM) components to stabilize and repair damaged tissue. Moreover, myofibroblasts contribute to tissue contraction and wound closure in later stages of the wound healing process via their inherent contractile function. In contrast to physiological wound healing, inflammation and ECM production are not self-limiting in IPF. As a consequence this leads to a continuous deposition of ECM, which finally results in progressive lung stiffening and the destruction of lung architecture. Indeed, ECM biomarkers can be used to determine the onset of the treatment of PF-ILD, see WO2017/207643. On the molecular level the pathogenesis of IPF is orchestrated by a multitude of pro-fibrotic mediators and signaling pathways. Besides TGFβ, which plays a central role in IPF due to its potent pro-fibrotic effects, tyrosine kinase signaling and elevation of various corresponding growth factors like e.g. platelet-derived growth factor (PDGF) and fibroblast growth factor (FGF) contribute to the pathogenesis of IPF.

In recent years several drugs have been clinically tested for the treatment of IPF. However, so far only two drugs, Pirfenidone (Esbriet®; Roche/Genentech) and Nintedanib (Ofev®; Boehringer Ingelheim), showed convincing therapeutic efficacy by slowing down disease progression as demonstrated by reduced rates of lung function decline. Despite these encouraging results, the medical need in IPF is still high and additional therapies with improved efficacy and ideally disease modifying potential are urgently needed. While investigations on the efficacy of Nintedanib in PF-ILDs other than IPF are ongoing, current treatment strategies mainly include corticosteroids and T- and B-cell targeted drugs (e.g. azathioprine, cyclophosphamide, methotrexate, mycophenolate mofetil), however, with limited success, again demonstrating a high demand for innovative therapeutic approaches.

### FIELD OF THE INVENTION

Due to the plethora of pathways involved in the pathogenesis of IPF and other fibrosing ILDs, multi-target therapies aiming to simultaneously modulate various disease mechanisms are likely to be most effective. However, respective approaches are difficult to implement by classical pharmacological strategies using small molecule compounds (NCEs) or biologicals (NBEs) like e.g. monoclonal antibodies, since both modalities are typically designed to specifically inhibit or activate a single drug target or a small set of closely related molecules. To enable multi-targeted therapies for PF-ILDs, microRNAs (miRNAs) represent a novel and highly attractive target class based on their ability to control and fine-tune entire signaling pathways or cellular mechanisms under physiological and pathophysiological conditions by regulating mRNA expression levels of a specific set of target genes. miRNAs are small non-coding RNAs, which are transcribed as pre-cursor molecules (pri-miRNAs). Inside the nucleus pri-miRNAs undergo a first maturation step to produce so called pre-miRNAs, which are characterized by a smaller hairpin structure. Following nuclear export, pre-miRNAs undergo a second processing step mediated by the Dicer enzyme, thereby generating two single strands of fully maturated miRNAs of approximately 22 nucleotides in length. To exert their gene regulatory function, mature miRNAs are incorporated into the RNA Induced Silencing Complex (RISC) to enable binding to miRNA binding sites positioned within the 3′-UTR of target mRNAs. Upon binding, miRNAs induce destabilization and cleavage of target mRNAs and/or modulate gene expression by inhibition of protein translation of respective mRNAs. To date more than 2000 miRNAs have been discovered in humans, which potentially regulate up to 30% of the transcriptome (Hammond S M, 2015).

The present invention discloses the identification of miRNAs involved in the pathogenesis of fibrosing lung disease and methods for the treatment of PF-ILD by functional modulation of respective miRNAs in PF-ILD patients, in particular IPF patients, using viral vectors, in particular an Adeno-associated virus (AAV). The present invention focusses on the treatment of humans though mammals of any kind, especially companion animal mammals, such as horses, dogs and cats are also within the realm of the invention.

### BRIEF SUMMARY OF THE INVENTION

Treatment of patients with moderate (Child Pugh B) and severe (Child Pugh C) hepatic impairment with Ofev is not recommended (see EPAR). Esbriet must not be used by patients already taking fluvoxamine (a medicine used to treat depression and obsessive compulsive disorder) or patients with severe liver or kidney problems (see EPAR). Thus, there is still a high medical need for PF-ILD patients, and in particular for IPF patients that have severe liver and kidney problems. It is an object of this invention to provide treatment alternatives. An alternative object of the invention is to provide treatment alternatives that may be eligible even for the patient group that cannot benefit from the existing therapies. While Esbriet and Ofev have shown convincing efficacy in clinical trials, also side effects are associated that potentially limit the options for a combined therapy of both drugs (see both EPARs). Thus, there is still a high medical need for PF-ILD and in particular IPF treatments with less side effects or at least with side effects different from those seen with Ofev or Esbriet, so that combined therapy with either Esbriet or Ofev may be viable option to increase the overall treatment efficacy. It is an object of the invention to provide treatment alternatives with a different risk/benefit profile compared to the established treatment options, e.g. with lesser side effects or with different side effects compared to the established treatment options. While Esbriet and Ofev are intended for oral, i.e. systemic use, there is still a need for a treatment option that can be administered by local administration or both via local and systemic routes. It is an alternative object of the invention to pros vide a treatment option that can be administered by local administration or both via local and systemic routes.

The present invention relates in one aspect to therapeutic agents, i.e. viral vectors and miRNA inhibitors or miRNA mimetics, for the treatment of PF-ILD in general and IPF in particular.

The viral vectors according to the invention stop or slow one or more aspects of the tissue transformation seen in PF-ILD and in particular IPF, such as the ECM deposits, by modulating miRNA function and thus stop or slow the decline in forced vital capacity seen in these diseases (see WO2017/207643 and references).

The viral vectors according to the invention may be administered to the patient via local (intranasal, intratracheal, inhalative) or systemic (intravenous) routes. Especially AAV vectors can target the lung quite efficiently, have a low antigenic potential and are thus particularly suitable also for systemic administration.

From a therapeutic perspective, miRNA function can be modulated by delivering miRNA mimetics to increase effects of endogenous miRNAs, which are downregulated under fibrotic conditions, or by delivering molecules to block miRNAs or to reduce their availability by so-called anti-miRs or miRNA sponges, thus inhibiting functionality of endogenous miRNAs, which are upregulated under pathological conditions.

Moreover, miRNAs described in the present invention, which are upregulated, might also exert protective functions as part of a natural anti-fibrotic response. However, this effect is apparently not sufficient to resolve the pathology on its own. Therefore, in specific cases, delivery of a miRNA mimetic for a sequence, which is already elevated under fibrotic conditions, can potentially further enhance its anti-fibrotic effect, thereby offering an additional model for therapeutic interventions.

Based on the fact that miRNAs orchestrate the simultaneous regulation of multiple target genes, viral vector mediated modulation of miRNA function represents an attractive strategy to enable multi-targeted therapies by affecting different disease pathways. The lung-fibrosis associated miRNAs described in the present invention distinguish from previously identified miRNAs by modulating different sets of target genes, thereby offering potential for improved therapeutic efficacy.

In the present invention a set of miRNAs associated with lung fibrosis has been identified by in-depth characterization and computational analysis of two disease-relevant animal models, in particular, Bleomycin-induced lung injury, characterized by a patchy, acute inflammation-driven fibrotic phenotype and AAV-TGFβ1 induced fibrosis that is reminiscent of the more homogenous NSIP pattern. Longitudinal transcriptional profiles of miRNAs and mRNAs as well as functional data have been generated to enable the identification of disease-associated miRNAs. Additionally, high confidence miRNA-mRNA regulatory relationships have been built based on sequence and expression anti-correlation, allowing for characterization of miRNAs in the context of the disease models based on their target sets. To further substantiate these findings, synthetic RNA oligonucleotide mimetics of selected miRNA candidates (mir-10a, mir-181a, mir-181b, mir-212-5p) were generated and used for transient transfection experiments in cellular fibrosis models in primary human lung fibroblasts, primary human bronchial airway epithelial cells and A549 cells. By investigating the effect of transiently transfected miRNAs on major aspects of TGFβ-induced fibrotic remodeling (inflammation, proliferation, fibroblast to myofibroblast transition (FMT), epithelial to mesenchymal transition (EMT)) the predicted anti-fibrotic effects of the selected miRNAs could be confirmed. Finally, to translate these findings into clinical applications, novel therapeutic approaches for fibrosing lung diseases to enable modulation of PF-ILD associated miRNAs by using viral gene delivery based on Adeno-associated virus (AAV) vectors are described.

The miRNA inhibitors or miRNA mimetics according to the invention stop or slow one or more aspects of the tissue transformation seen in PF-ILD and in particular IPF, such as the ECM deposits, by modulating miRNA function and thus stop or slow the decline in forced vital capacity seen in these diseases (see WO2017/207643 and references). Compared to viral vectors according to the invention, they have a different profile of side effects, such as a potentially lower antigenicity, thereby potentially allowing multiple treatments without immunosuppressive combined treatment.

By conducting a longitudinal in depth analysis of two disease-relevant animal models, namely the Bleomycin- and the AAV-TGFβ1-induced lung fibrosis model in mice, a novel set of 28 miRNAs has been identified. To select the most relevant miRNAs, the inventors developed a hit selection strategy based on systematic correlation analyses between gene expression profiling data and key functional disease parameters. Under consideration of the chronic nature of PF-ILDs the inventors describe expression of miRNAs, anti-miRs or miRNA sponges by viral vectors especially those based on Adeno-associated virus (AAV) as a novel therapeutic concept to enable long lasting expression of therapeutic nucleic acids for functional modulation of fibrosis-associated miRNAs.

### BRIEF DESCRIPTION OF THE DRAWINGS

FIG. 1 illustrates the study design. A total of 130 C57Bl/6 mice either received NaCl, 1 mg/kg Bleomycin or 2.5×10¹¹ vector genomes (vg) of either AAV6.2-stuffer control or AAV6.2-CMV-TGFβ1 vector by intratracheal administration. At each readout and sampling (RS) time point illustrated in the scheme, lung function measurement was performed and the wet lung weight was determined. The left lung was then used for histological assessment of fibrosis development and the right lung was lysed for the isolation of total lung RNA. RNA was applied to next generation sequencing in order to profile gene expression changes correlating with disease manifestation.

FIG. 2 shows data on the functional characterization of lung pathology. Mice were treated as described in FIG. 1 and fibrosis development was monitored. (A) Masson trichrome-stained histological lung sections from day 21 after administration demonstrate fibrosis manifestation evident from alveolar septa thickening, increased extracellular matrix deposition and presence of immune cells. The lower panel of images shows 10× magnified details of the upper panel of micrographs. (B) An increase in wet lung weight in AAV-TGFβ1 and Bleomycin treated animals indicates increased ECM deposition, leading to (C) strong impairment of lung function in fibrotic animals. Mean+/−SD, **p<0.01, ***p<0.001, relative to respective control treatment.

FIG. 3 summarizes results from the gene expression analysis. By performing parallel mRNA- and miRNA-sequencing, up- and down-regulated mRNAs (A) and miRNAs (B) were identified in both models at every time point analyzed. Cut-off criteria for identification of differentially expressed genes: P adj. (FDR)≤0.05, abs(log 2FC)≥0.5 (FC≥1.414). (C) mRNAs showing differential expression exclusively in one of the models were separated from mRNAs that were differentially expressed in both models (commonly DE) at each time point and applied to KEGG pathway enrichment analysis. The data show enrichment for acute inflammation (“cytokine-cytokine receptor interaction”) at early time points in the Bleomycin model but not the AAV model, whereas enrichment for fibrosis development (“ECM receptor interaction”) was observed in a time-dependent fashion in both models.

FIG. 4 provides an overview of the filtering process applied for identification of fibrosis-associated miRNAs. In a first step miRNAs correlating (C) or anti-correlating (AC) with lung function and/or lung weight in at least one of the two models were identified. Subsequently, correlated and anti-correlated miRNAs were filtered for candidates showing differential gene expression. By definition miRNAs were regarded as differentially expressed when expression level changes (P adj. (FDR)≤0.05, abs(log 2FC)≥0.5; up- or downregulation) were observed in at least one of the animal models at one or more time points. In a final step filtered miRNAs were assessed with regard to species conservation. miRNAs showing sequence identity in the seed region and an alignment score of at least 20 for the mature miRNA sequence between mouse and human were regarded as homologs, whereas the remaining miRNAs were categorized as mouse-specific and thus nonconserved. Finally, the resulting hit list was hand-curated by e.g. eliminating candidates with dissimilar or strongly fluctuating expression profiles, previously patented miRNAs and non-conserved upregulated miRNAs, because those could not be targeted in humans.

FIG. 5 A shows fibrosis-associated miRNAs identified by applying the filtering process as described in FIG. 4. Except for mmu-miR-30f and mmu-miR-7656-3p, for which no human homologs were identified, all miRNAs shown are species conserved (highly similar or identical). Mismatches to the human homolog are shown in bold face and underlined. Depicted sequences represent the processed and fully maturated miRNAs.

The closest human homologs of the mouse sequences that are highly similar (albeit not identical) are shown in FIG. 5 B.

The shown sequences are also compiled in a sequence listing. In case of contradictions between the sequence listing and FIGS. 5 A and B, FIG. 5 represents the authentic sequence.

FIG. 6 schematically illustrates the target prediction workflow. For the miRNA candidates listed in FIG. 5, mRNA targets were predicted by querying DIANA, MiRanda, PicTar, TargetScan and miRDB databases. mRNAs predicted by at least two out of five databases were considered and filtered further by the anticorrelation of expression between miRNA and mRNA measurements in the animal models. Predicted mRNAs whose longitudinal expression was anti-correlated (rho≤−0.6) with the expression of its corresponding miRNA were called putative targets. Subsequently, target lists were subjected to pathway enrichment analysis for functional characterization of the miRNA target spectrum.

FIG. 7 shows the characterization of miRNA function based on enrichment of predicted target sets. Predicted target sets for each miRNA underwent enrichment tests vs. reference gene sets from different sources. The table shows −log(p adj) of a subset of the selected set of miRNAs for a small subset of selected gene sets that are relevant in the context of pulmonary fibrosis. Higher values indicate stronger enrichment.

FIG. 8 describes vector designs to enable expression of miRNAs or miRNA targeting constructs. (A) Single miRNAs or combinations of miRNAs, which are downregulated under fibrotic conditions, can be expressed from vectors using Polymerase-II (Pol-II) or Polymerase-III (Pol-III) promoters. miRNA sequences can be expressed by using the natural backbone of a respective miRNA or embedded into a foreign miRNA backbone, thereby generating an artificial miRNA. In both cases miRNAs are expressed as precursor miRNAs (pri-miRNAs), which are processed inside the cell into mature miRNAs. Mechanistically, processed miRNAs selectively bind to miRNA binding sites positioned in the 3′-UTR of target genes thereby leading to reduced expression levels of fibrosis-associated genes via mRNA degradation and/or inhibition of protein translation. (B) Inhibition of endogenous miRNAs, which are upregulated under fibrotic conditions, can be achieved by expression of antisense-like molecules, so called anti-miRs. Respective sequences can be expressed from a shRNA backbone or from an artificial miRNA backbone by using Pol-II or Pol-III promoters. After intracellular processing, anti-miRs bind to pro-fibrotic target miRNAs, thereby blocking their functionality. (C) An alternative approach to inhibit pro-fibrotic miRNAs is the expression of mRNAs harboring miRNA-specific targeting sequences, so-called sponges. Upon expression using a Pol-II promoter, miRNA sponges lead to the sequestration of pro-fibrotic miRNAs, thereby inhibiting their pathological function.

FIG. 9 illustrates the generation of Adeno-associated virus (AAV) vectors for delivery of miRNA-expressing or miRNA-targeting constructs to the lung. Flanking of expression constructs by AAV inverted terminal repeats (ITRs) at the 5′- and the 3′-end enables packaging into AAV vectors. Various natural serotypes (AAV5, AAV6) or modified capsid variants (AAV2-L1, AAV6.2) have been described previously as highly potent vectors to enable efficient gene delivery to the lung via both, local (intranasal, intratracheal, inhalative) or systemic (intravenous) routes of administration.

FIG. 10 provides examples of AAV-mediated gene delivery to the lung by different AAV serotypes or capsid variants. (A) Immuno-histological staining of green fluorescent protein (GFP) expression in lung sections from C57BL/6J mice 2 weeks after intravenous injection of AAV2-L1-GFP (3×10¹¹ vg/mouse), a recently described AAV2 variant harboring a peptide insertion motive to enable lung-specific gene delivery following systemic administration (Körbelin J et al., 2016). No specific signals beyond background staining were observed in the PBS control group. Representative images from two mice (ms 1, ms 2) out of n=6 animals per group are shown. (B) Assessment of AAV2-L1 bio-distribution by in vivo imaging in FVB/N mice (Published data: Körbelin J et al., 2016). Lung-specific expression of firefly luciferase (fLuc) was observed 2 weeks after intravenous injection of fLuc-expressing AAV2-L1 vector at a dose of 5×10¹⁰ vg/mouse. (C) Ex vivo imaging of mouse lungs prepared from C57BL/6J mice 2 weeks after intra-tracheal instillation of fLuc-expressing AAV5 vectors (2.9×10¹⁰ vg/mouse) or PBS as a negative control. Quantitative lung transduction was observed in AAV5-fLuc treated animals by detecting light emission resulting from fLuc-positive cells in the luminescence (Lum) channel. Brightfield (BF) images of prepared lungs are shown in the upper panel. Representative images from two mice (ms 1, ms 2) out of n=4 animals per group are shown (D) Analysis of AAV6.2-mediated lung delivery in Balb/c mice three weeks after intratracheal application of GFP-expressing AAV6.2 vectors at a dose of 3×10¹¹ vg/mouse. Micrographs of histological lung sections show direct GFP fluorescence (right) and immuno-histological analysis of GFP expression (left). No specific signals beyond background staining were observed in the PBS control group. Representative images of n=5 animals per group are shown.

FIG. 11 provides examples of different miRNA expression cassettes. A) Vector map of CMV-mir181a-scAAV (Double stranded AAV vector genome for simultaneous expression of a cDNA (eGFP) and a miRNA) and CMV-mir181a-mir181b-mir10a-scAAV (Double stranded AAV vector genome for simultaneous expression of three miRNAs). B) Illustration of different miRNA-designs in the miR-E backbone using mir-181b-5p as an example. The first two examples show mir-181b-5p integrated as fully matured miRNA (23 nt) at the passenger or guide position in the miR-E backbone using perfectly matched complementary strands. The second example illustrates a construct design integrating mir-181b as naturally occurring pre-miRNA into the miR-E backbone. Predicted 2D-structure of mir181b derived from mirBase (http://mirbase.org/).

FIG. 12 shows knock-down efficiencies of miR181a-5p and miR212-5p in the mir-E backbone on GFP expression construct having the corresponding target sequences in the 3′UTR. HEK-293 cells were transiently transfected with the GFP expression construct in combination with a plasmid encoding one of the miRNAs. GFP fluorescence was measured 72 h after transfection. Positive control is an optimal mir-E construct whereas the 3′UTR of the GFP construct is lacking the target sequence for the negative control.

FIG. 13 shows the basal miRNA expression of human orthologues of the murine candidate miRNAs in normal human lung fibroblasts (NHLFs), measured by using small RNA-sequencing with n=6 replicates. Expression levels are depicted as counts per million (cpm). Arrows mark miRNA candidates of particular interest, which were selected for further functional characterization.

FIG. 14 shows the effect of miRNAs on inflammatory IL6 expression in unstimulated or TGFβ1-stimulated A549 cells. (A) IL6 expression was assessed by transfection of cells with either miRNA control constructs (Ctrl) or mimetic of the depicted miRNA candidates at 2 nM concentration. 24 hours after transfection cells were stimulated with 5 ng/mL TGFβ1 for another 24 hours. Extracted RNA was then reversely transcribed to cDNA and IL6 gene expression was measured by qPCR. (B) Cells were transfected and stimulated as described in (A) and secreted IL6 protein was detected by ELISA measurements in the cell supernatant. Expression levels are expressed relative to the unstimulated miRNA control construct (Ctrl). Triple=co-transfection of miR-10a-5p, miR-181a-5p and miR-181b-5p. n=3 experiments, mean±SD. *p<0.05, **p<0.01, ***p<0.001 (miRNA candidate vs. Ctrl).

FIG. 15A shows the effect of single miRNAs and their combination on the epithelial-mesenchymal transition (EMT) of normal human bronchial epithelial cells (NHBECs). EMT was assessed by transfection of cells with either miRNA control constructs (Ctrl), mimetic of the depicted miRNA candidates at 2 nM concentration or their combination at 4 nM or 12 nM, as illustrated, followed by stimulation with 5 ng/mL TGFβ1. E-cadherin (a marker of epithelial cells) was immuno-stained 72 h later, quantified by high-content cellular imaging, normalized by the number of detected cells and depicted here as fold change between miRNA candidates and control. An increase in E-cadherin is indicative of the maintenance of epithelial characteristics and therefore considered anti-fibrotic. n=4 replicates, mean±SD. *p<0.05, **p<0.01 (miRNA candidate vs. Ctrl). SSMD: strictly standardized mean difference; #: |SSMD|>2, ##: |SSMD|>3, ###: |SSMD|>5.

FIG. 15B provides dose/response experiments of single miRNAs and their combination on the epithelial-mesenchymal transition (EMT) of normal human bronchial epithelial cells (NHBECs). EMT was assessed by transfection of cells with either miRNA control constructs (Ctrl), mimetic of the depicted miRNA candidates at rising concentrations (0.25 nM, 0.5 nM, 1 nM, 2 nM 4 nM, 8 nM, 16 nM). The given concentrations are total concentrations. For double or triple miRNA combinations, the total concentration has to be divided by two or three, respectively, to gain the concentration of involved single miRNA mimetic. Cells were stimulated with 5 ng/mL TGFβ1. E-cadherin (a marker of epithelial cells) was immuno-stained 72 h later, quantified by high-content cellular imaging, normalized by the number of detected cells and depicted here as fold change between miRNA candidates and control. An increase in E-cadherin is indicative of the maintenance of epithelial characteristics and therefore considered anti-fibrotic. n=4 replicates, mean±SD. *p<0.05, **p<0.01 (miRNA candidate vs. Ctrl).

FIG. 16 shows the effect of miRNAs on inflammatory IL6 expression in unstimulated or TGFβ1-stimulated normal human lung fibroblasts (NHLFs). IL6 expression was assessed by transfection of cells with either miRNA control constructs (Ctrl) or mimetic of the depicted miRNA candidates at 2 nM concentration. 24 hours after transfection cells were stimulated with 5 ng/mL TGFβ1 for another 24 hours. Extracted RNA was then reversely transcribed to cDNA and IL6 gene expression was measured by qPCR. n=3 replicates, mean±SD. *p<0.05, **p<0.01, ***p<0.001 (miRNA candidate vs. Ctrl).

FIG. 17 shows the effect of miRNAs on the proliferation of unstimulated or TGFβ1-stimulated normal human lung fibroblasts (NHLFs). Proliferation was assessed by transfection of cells with either miRNA control constructs (Ctrl) or mimetic of the depicted miRNA candidates at 2 nM concentration, followed by stimulation with 5 ng/mL TGFβ1. Proliferation was measured using a spectrophotometric enzymatic WST-1 proliferation assay that measures cellular metabolic activity (mitochondrial dehydrogenase) as a direct correlate of the number of cells. n=3 replicates, mean±SD. *p<0.05, **p<0.01 (miRNA candidate vs. Ctrl).

FIG. 18 shows the effect of single miRNAs and their combination on the fibroblast-to-myofibroblast transition (FMT) of normal human lung fibroblast (NHLFs). FMT was assessed by transfection of cells with either miRNA control constructs (Ctrl), mimetic of the depicted miRNA candidates at 2 nM concentration or their combination at 4 nM or 12 nM, as illustrated, followed by stimulation with 5 ng/mL TGFβ1. Collagen type 1 α1 (a marker of myofibroblasts), was immuno-stained 72 h later, quantified by high-content cellular imaging, normalized by the number of detected cells and depicted here as fold change between miRNA candidates and control. A decrease in collagen is indicative of a loss of myofibroblast characteristics and therefore considered anti-fibrotic. n=2 donors (4 replicates each), mean±SD. *p<0.05, **p<0.01 (miRNA candidate vs. Ctrl). SSMD: strictly standardized mean difference; #: |SMD|>2.

FIG. 19 shows the effect of single miRNA-181a and miR-212-5p on collagen 1 deposition of normal and IPF human lung fibroblasts. Collagen 1 deposition was assessed by transfection of cells with either miRNA control constructs (Ctrl), mimetic of the depicted miRNA candidates at rising concentrations (0.25 nM, 0.5 nM, 1 nM, 2 nM 4 nM, 8 nM, 16 nM). Cells were stimulated with 5 ng/ml TGFβ1. Collagen type 1 α1, was immunostained 72 h later, quantified by high-content cellular imaging, normalized by the number of detected cells and depicted here as fold change between miRNA candidates and control. A decrease in collagen is indicative of a loss of myofibroblast characteristics and therefore considered anti-fibrotic. n=7 donors, mean±SD. Two-way ANOVA, Dunnett's multiple comparison.

FIG. 20 shows the effect of miRNA 181a-5p and miR212-5p on the expression of different collagen sub-types in lung fibroblasts. A) Col1a1 and B) Col5a1 protein expression and C) Col3a1 mRNA expression was assessed by transfection of cells with either miRNA control constructs (Ctrl), mimetic of the depicted miRNA candidates at 2 nM (single miRNA) or miRNA combination with 2+2 nM. Cells were stimulated with 5 ng/ml TGFβ1. Collagen type 1α1 and 5α1, was immuno-stained with Western Blot technique, 72 h later and quantified by densitometry. Collagen expression was normalized to GAPDH expression. Col3a1 was quantified 24 h later via RT-qPCR. Col 3a1 mRNA expression was normalized with the delta/delta cT method to HPRT mRNA. A decrease in collagens is indicative for fibrosis reduction. Depicted are fold changes between miRNA candidates and control+TGF β1 for A (n=5) and B (n=3) or fold changes between miRNA candidates and miRNA control+TGF β1 for C (n=4). Depicted are means±SD. * p<0.05, ** p<0.01, One-way-ANOVA, Tukey's multiple comparisons test.

FIG. 21 shows the effect of miRNA 181a-5p and miR212-5p on the mRNA expression of Col1a1 on lung fibroblasts in an A549 epithelial-fibroblast co-culture. Col1a1 mRNA expression was assessed by transfection of cells with either miRNA control constructs (Ctrl), mimetic of the depicted miRNA candidates at 2 nM. A549 cells were seeded to 100% confluence on a permeable stimulated cell filter, with sub-cultured lung fibroblasts. A549 cells and fibroblast were separated by the filter, but allowing the flow of A549 secreted factors to the fibroblasts. Only A549 cells were stimulated with 5 ng/ml TGFβ1, whereas sub-seeded lung fibroblasts were not stimulated with exogenous TGFβ1. Collagen type 1a1 mRNA was quantified in lung fibroblasts 24 h later via RT-qPCR. Col 1a1 mRNA expression was normalized with the delta/delta cT method to HPRT mRNA. A decrease in collagens is indicative for fibrosis reduction. Depicted are fold changes between miRNA candidates and miRNA control+TGF β1 (n=3). Depicted are means±SD. * p<0.05, ** p<0.01, One-way-ANOVA, Tukey's multiple comparisons test.

### SUMMARY OF THE INVENTION

The invention relates to a viral vector comprising: a capsid and a packaged nucleic acid, wherein the nucleic acid augments either (i) the miRNA of Seq ID No. 15 or (ii) miRNA downregulated in a Bleomycin-induced lung fibrosis model or in an AAV-TGFβ1-induced lung fibrosis model, wherein the miRNA comprises miRNA of Seq ID 17, 18 or 19, or (iii) both (i) and (ii). In one embodiment, the miRNA(s) that are downregulated in a Bleomycin-induced lung fibrosis model or in an AAV-TGFβ1-induced lung fibrosis model and which are augmented by the packaged nucleic acid comprise the miRNA of Seq ID No. 19. In another embodiment, the one or more miRNAs which are augmented by the packaged nucleic acid comprise the miRNA of Seq ID No. 19 and the miRNA of Seq ID No. 18 or the miRNA of Seq ID No. 19 and the miRNA of Seq ID No. 17. Augmentation in this context means that the level of the respective miRNA in the transduced cell is increased as a result of the transduction of the target cell, which is preferably a lung cell.

The invention further relates to a viral vector comprising: a capsid and a packaged nucleic acid, wherein the nucleic acid augments either (i) the miRNA of Seq ID No. 15 or (ii) miRNA downregulated in a Bleomycin-induced lung fibrosis model or in an AAV-TGFβ1-induced lung fibrosis model, wherein the miRNA comprises miRNA of Seq ID 17, 18 or 19, or (iii) both (i) and (ii) and wherein the nucleic acid further inhibits miRNA selected form the group consisting of miRNAs of Seq ID No 1, 2, 3, 4, 5, 6, 7, 8, 9, 10, 11, 12, 13, 14, and 16 or the closest human homolog of respective sequences in case of miRNAs with partial sequence conservation.

Inhibition in this context means that the function of the respective miRNA in the transduced cell is reduced or abolished by complementary binding as a result of the transduction of the target cell.

In one embodiment of the invention relates to a viral vector comprising: a capsid and a packaged nucleic acid that codes for one or more miRNA that are downregulated in a Bleomycin-induced lung fibrosis model or in an AAV-TGFβ1-induced lung fibrosis model:

a) In one preferred embodiment, the one or more miRNA encoded by the packaged nucleic acid comprise the miRNA of Seq ID No. 15. In another embodiment, the one or more miRNAs encoded by the packaged nucleic acid comprise (i) the miRNA of Seq ID No. 15 and the miRNA of Seq ID No. 17 or (ii) the miRNA of Seq ID No. 15 and the miRNA of Seq ID No. 19 or (iii) the miRNA of Seq ID No. 15 and the miRNA of Seq ID No. 19 and the miRNA of Seq ID No. 18, or (iv) the miRNA of Seq ID No. 15 and the miRNA of Seq ID No. 17 and the miRNA of Seq ID No. 18, or (v) the miRNA of Seq ID No. 15 and the miRNA of Seq ID No. 17 and the miRNA of Seq ID No. 19.
b) In one embodiment, the one or more miRNA encoded by the packaged nucleic acid comprise the miRNA of Seq ID No. 19. In another embodiment, the one or more miRNAs encoded by the packaged nucleic acid comprise (i) the miRNA of Seq ID No. 19 and the miRNA of Seq ID No. 18 or (ii) the miRNA of Seq ID No. 19 and the miRNA of Seq ID No. 17 or (iii, preferred) the miRNA of Seq ID No. 19 and the miRNA of Seq ID No. 17 and the miRNA of Seq ID No. 18.
c) In one embodiment, the one or more miRNA encoded by the packaged nucleic acid comprise the miRNA of Seq ID No. 17. In another embodiment, the one or more miRNAs encoded by the packaged nucleic acid comprise (i) the miRNA of Seq ID No. 17 and the miRNA of Seq ID No. 18 or (ii) the miRNA of Seq ID No. 17 and the miRNA of Seq ID No. 19.

It is understood that the nucleic acid usually comprises coding and non-coding regions and that the encoded miRNA downregulated in a Bleomycin-induced lung fibrosis model or in an AAV-TGFβ1-induced lung fibrosis model results from transcription and subsequent maturation steps in target cell transduced by the viral vector.

It is understood that the nucleic acid usually comprises coding and non-coding regions and that the encoded RNA inhibiting the function of one or more miRNA that is upregulated in a Bleomycin-induced lung fibrosis model or in an AAV-TGFβ1-induced lung fibrosis model results from transcription and potentially, but not necessarily, subsequent maturation steps in target cell transduced by the viral vector.

Viral vectors according to the present invention are selected so that they have the potential to transduce lung cells. Non-limiting examples of viral vectors that transduce lung cells include, but are not limited to lentivirus vectors, adenovirus vectors, adeno-associated virus vectors (AAV vectors), and paramyxovirus vectors. Among these, the AAV vectors are particularly preferred, especially those with an AAV-2, AAV-5 or AAV-6.2 serotype. AAV vectors having a recombinant capsid protein comprising Seq ID No. 29, 30 or 31 are particularly preferred (see WO 2015/018860). In one embodiment, the AAV vector is of the AAV-6.2 serotype and comprises a capsid protein of the sequence of Seq ID No. 82.

The sequence coding for the miRNA thereby augmenting its function and the sequence coding for an RNA that inhibits the function of one or more miRNA may or may not be within the same transgene.

In one embodiment, the invention relates to viral vector comprising: a capsid and a packaged nucleic acid comprising one or more transgene expression cassettes comprising:
a transgene that codes for one or more miRNAs selected from the group consisting of the miRNAs of Seq ID Nos. 15, 17, 18 and 19,
and a transgene that codes for an RNA that inhibits the function of one or more miRNAs selected form the group consisting of the miRNAs of Seq ID Nos. 1, 2, 3, 4, 5, 6, 7, 8, 9, 10, 11, 12, 13, 14, 16, 34, 35 and 36.

Accordingly, the transgene that codes for a miRNA thereby augmenting its level and the transgene that codes for an RNA that inhibits the function of one or more miRNA are contained in different expression cassettes.

In one embodiment, the invention relates to a viral vector comprising: a capsid and a packaged nucleic acid comprising one or more transgene expression cassettes comprising a transgene that codes
for one or more miRNAs selected from the group consisting of the miRNAs of Seq ID Nos. 15, 17, 18 and 19, and further codes
for an RNA that inhibits the function of one or more miRNAs selected from the group consisting of the miRNAs of Seq ID Nos. 1, 2, 3, 4, 5, 6, 7, 8, 9, 10, 11, 12, 13, 14, 16, 34, 35 and 36.

Accordingly, one transgene codes for both a miRNA thereby augmenting its function and for a RNA that inhibits the function of one or more miRNA.

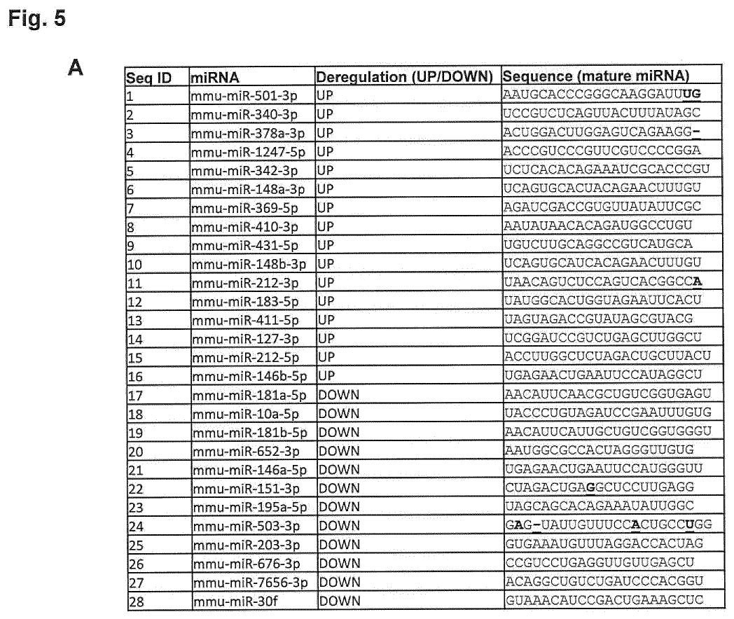

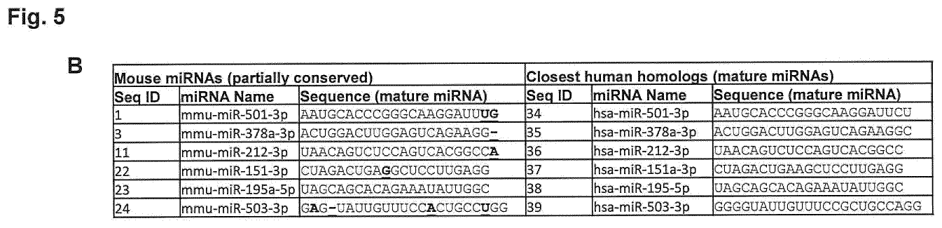

In another embodiment of the invention a viral vector is provided, wherein the miRNA that is downregulated in a Bleomycin-induced lung fibrosis model or in an AAV-TGFβ1-induced lung fibrosis model is selected from the group consisting of miRNAs of Seq ID No. 17, 18, 19, 20, 21, 22, 23, 24, 25, 26, 27 and 28 or the closest human homolog of respective sequences in case of miRNAs with partial sequence conservation. In this group, the conserved miRNA, namely 17, 18, 19, 20, 21, 22, 24, 25, 26 or their closest human homolog are most preferred. The closest human homolog of the respective sequences is shown in FIG. 5 B.

In another preferred embodiment of the invention a viral vector is provided, wherein the miRNA that is downregulated in a Bleomycin-induced lung fibrosis model or in an AAV-TGFβ1-induced lung fibrosis model, is selected from the group consisting of miRNAs of Seq ID No. 17, 18, 19, and 20 (mmu-miR-181a-5p, mmu-miR-10a-5p, mmu-miR-181b-5p, and mmu-miR-652-3p, respectively) and most preferred is Seq ID No. 17 (mmu-miR-181a-5p).

In a further embodiment of the invention a viral vector is provided, wherein the packaged nucleic acid codes for a miRNA having the sequence of Seq ID No. 17, and for a miRNA having the sequence of Seq ID No. 18 and for a miRNA having the sequence of Seq ID No. 19.

In a further embodiment of the invention a viral vector is provided, wherein the packaged nucleic acid codes for four miRNA having the sequence of Seq ID No. 17, 18, 19, and 20.

In a further embodiment of the invention a viral vector is provided, wherein the nucleic acid has an even number of transgene expression cassettes and optionally the transgene expression cassettes comprising (or consisting of) a promotor, a transgene and a polyadenylation signal, wherein promotors or the polyadenylation signals are positioned opposed to each other.

The viral vector is a recombinant AAV vector in one embodiment of the invention and has either the AAV-2 serotype, AAV-5 serotype or the AAV-6.2 serotype in other embodiments of the invention.

In a different embodiment of the invention a viral vector is provided, wherein the capsid comprises a first protein that comprises the sequence of Seq ID No. 29 or 30 (see WO 2015/018860).
i) In a further embodiment of the invention a viral vector is provided, wherein the capsid comprises a first protein that is 80% identical, more preferably 90%, most preferred 95% to a second protein having the sequence of Seq ID No. 82, whereas one or more gaps in the alignment between the first protein and the second are allowed
ii) In a different embodiment of the invention a viral vector is provided, wherein the capsid comprises a first protein that is 80% identical, more preferably 90%, most preferred 95% identical to a second protein of Seq ID No. 82 whereas a gap in the alignment between the first protein and the second protein is counted as a mismatch.
iii) In a different embodiment of the invention a viral vector is provided, wherein the capsid comprises a first protein that is 80% identical, more preferably 90%, most preferred 95% identical to a second protein of Seq ID No. 82, whereas no gaps in the alignment between the first protein and the second protein are allowed.

For all embodiments (i) to (iii): For the determination of the identity between a first protein and a reference protein, any amino acid that has no identical counterpart in the alignment between the two proteins counts as mismatch (including overhangs with no counterpart). For the determination of identity, the alignment is used which gives the highest identity score.

The packaged nucleic acid may be single or double stranded. An alternative especially for AAV vectors is to use self-complementary design, in which the vector genome is packaged as a double-stranded nucleic acid. Although the onset of expression is more rapid, the packaging capacity of the vector will be reduced to approximately 2.3 kb, see Naso et al. 2017, with references.

A further aspect of the invention is one of the described viral vectors for use in the treatment of a disease selected from the group consisting of PF-ILD, IPF, connective tissue disease (CTD)-associated ILD, rheumatoid arthritis ILD, chronic fibrosing hypersensitivity pneumonitis (HP), idiopathic non-specific interstitial pneumonia (iNSIP), unclassifiable idiopathic interstitial pneumonia (IIP), environmental/occupational lung disease, systemic sclerosis ILD and sarcoidosis, and fibrosarcoma.

Delivery Strategies for Recombinant AAV Therapeutics are also referred in e.g. Naso et al, 2017.

A double stranded plasmid vector comprising said AAV vector genome is a further embodiment of the invention.

A further embodiment of the invention relates to this miRNA inhibitor for use as a medicinal product.

A further embodiment of the invention is a miRNA mimetic for use in a method of prevention and/or treatment of a fibroproliferative disorder, wherein miRNA has a sequence selected from the group consisting of Seq ID No. 15, 17, 18, 19, 20, 21, 22, 23, 24, 25, 26, 27, 37, 38 and 39, preferably selected from the group consisting of Seq ID No. 15, 17 and 19, and most preferred has the sequence of Seq ID No. 15 or 19. In one embodiment, a miRNA mimetic is provided for use in a method of prevention and/or treatment of a fibroproliferative disorder, such as IPF or PF-ILD, wherein miRNA has the sequence of Seq ID No. 19. The prevention and/or treatment preferably further comprises the administration of a mimetic for a miRNA having the sequence of Seq ID No. 17 or of a mimetic for a miRNA having the sequence of Seq ID No. 18. Even more preferably, the prevention and/or treatment comprises the administration of a mimetic for a miRNA having the sequence of Seq ID No. 19, a mimetic for a miRNA having the sequence of Seq ID No. 17 and of mimetic for a miRNA having the sequence of Seq ID No. 18.

Likewise, in a further embodiment a miRNA mimetic is provided for use in a method of prevention and/or treatment of a fibroproliferative disorder, such as IPF or PF-ILD, wherein the miRNA has the Seq ID No. 15. The prevention and/or treatment preferably further comprises the administration of a mimetic for a miRNA having the sequence of Seq ID No. 17 or of a mimetic for a miRNA having the sequence of Seq ID No. 19. Even more preferably,
the prevention and/or treatment comprises the administration of a mimetic for a miRNA having the sequence of Seq ID No. 15, a mimetic for a miRNA having the sequence of Seq ID No. 19 and of a mimetic for a miRNA having the sequence of Seq ID No. 18, or
the prevention and/or treatment comprises the administration of a mimetic for a miRNA having the sequence of Seq ID No. 15, a mimetic for a miRNA having the sequence of Seq ID No. 17 and of a mimetic for a miRNA having the sequence of Seq ID No. 18, or
the prevention and/or treatment comprises the administration of a mimetic for a miRNA having the sequence of Seq ID No. 15, a mimetic for a miRNA having the sequence of Seq ID No. 17 and of a mimetic for a miRNA having the sequence of Seq ID No. 19.

A further embodiment of the invention is (i) a miRNA mimetic of a miRNA having the sequence of Seq ID No. 15, or (ii) a miRNA mimetic of a miRNA having the sequence of Seq ID No. 17, or (iii) a miRNA mimetic of a miRNA having the sequence of Seq ID No. 18, or (iv) a miRNA mimetic of a miRNA having the sequence of Seq ID No. 19, for the treatment of a fibroproliferative disorder such as IPF or PF-IL, and a pharmaceutical composition comprising one or more of said miRNA mimetics (i) to (iv) and a pharmaceutical-acceptable carrier or diluent.

Further embodiments of the invention are miRNA mimetics of miRNA 212-5p (Seq ID No. 15), miRNA 181a-5p (Seq ID No. 17), miRNA 181b-5p (Seq ID No. 19), and miRNA 10a (Seq ID No. 18), respectively for use in the treatment of a fibroproliferative disorder, and wherein the miRNA mimetic is an oligomer of nucleotides that consists of the sequence selected form the group consisting of Seq ID No. 15, Seq ID No. 17, Seq ID No. 19, and Seq ID No. 18, respectively with the following proviso:
the oligomer optionally comprises nucleotides with chemical modifications leading to non-naturally occurring nucleotides that show the base-pairing behavior at the corresponding position (AU and GC) as determined by the sequence of the respective miRNA;
the oligomer optionally comprises nucleotide analogues that show the basepairing behavior at the corresponding position (AU and GC) as determined by the sequence of the respective miRNA;
the oligomer is optionally lipid conjugated to facilitate drug delivery.

Further embodiments of the invention are miRNA mimetics of miRNA 212-5p (Seq ID No. 15), miRNA 181a-5p (Seq ID No. 17), miRNA 181b-5p (Seq ID No. 19), and miRNA 10a (Seq ID No. 18), respectively for use in the treatment of a fibroproliferative disorder, and wherein the miRNA mimetic is an oligomer of nucleotides that consists of the sequence selected form the group consisting of Seq ID No. 15, Seq ID No. 17, Seq ID No. 19, and Seq ID No. 18, respectively with the following proviso:
the oligomer optionally comprises nucleotides with chemical modifications leading to non-naturally occurring nucleotides that show the base-pairing behavior at the corresponding position (AU and GC) as determined by the sequence of the respective miRNA;
the oligomer is optionally lipid conjugated to facilitate drug delivery.

Further embodiment of the invention are miRNA mimetics of miRNA 212-5p (Seq ID No. 15), miRNA 181a-5p (Seq ID No. 17), miRNA 181b-5p (Seq ID No. 19), and miRNA 10a (Seq ID No. 18), respectively for use in the treatment of a fibroproliferative disorder, and wherein the miRNA mimetic is an oligomer of nucleotides that consists of the sequence selected form the group consisting of Seq ID No. 15, Seq ID No. 17, Seq ID No. 19, and Seq ID No. 18, respectively with the following proviso:
the oligomer optionally comprises nucleotide analogues that show the basepairing behavior at the corresponding position (AU and GC) as determined by the sequence of the respective miRNA;
the oligomer is optionally lipid conjugated to facilitate drug delivery.

In case, the miRNA mimetics are not delivered being packed in lipid based nano particles (LNPs), it is preferred that the oligomer mentioned in the proviso is lipid conjugated to facilitate drug delivery.

Further embodiments of the invention are miRNA mimetics of miRNA 212-5p (Seq ID No. 15), miRNA 181a-5p (Seq ID No. 17), miRNA 181b-5p (Seq ID No. 19), and miRNA 10a (Seq ID No. 18), respectively for use in the treatment of a fibroproliferative disorder, and wherein the miRNA mimetic is an oligomer of nucleotides that consists of the sequence selected form the group consisting of Seq ID No. 15, Seq ID No. 17, Seq ID No. 19, and Seq ID No. 18, respectively with the following proviso:
the oligomer optionally comprises nucleotides with chemical modifications leading to non-naturally occurring nucleotides that show the base-pairing behavior at the corresponding position (AU and GC) as determined by the sequence of the respective miRNA;
the oligomer optionally comprises nucleotide analogues that show the basepairing behavior at the corresponding position (AU and GC) as determined by the sequence of the respective miRNA.

Further embodiments of the invention are miRNA mimetics of miRNA 212-5p (Seq ID No. 15), miRNA 181a-5p (Seq ID No. 17), miRNA 181b-5p (Seq ID No. 19), and miRNA 10a (Seq ID No. 18), respectively for use in the treatment of a fibroproliferative disorder, and wherein the miRNA mimetic is an oligomer of nucleotides that consists of the sequence selected form the group consisting of Seq ID No. 15, Seq ID No. 17, Seq ID No. 19, and Seq ID No. 18, respectively, with the following proviso:
the oligomer optionally comprises nucleotides with chemical modifications leading to non-naturally occurring nucleotides that show the base-pairing behavior at the corresponding position (AU and GC) as determined by the sequence of the respective miRNA.

Further embodiments of the invention are miRNA mimetics of miRNA 212-5p (Seq ID No. 15), miRNA 181a-5p (Seq ID No. 17), miRNA 181b-5p (Seq ID No. 19), and miRNA 10a (Seq ID No. 18), respectively for use in the treatment of a fibroproliferative disorder, and wherein the miRNA mimetic is an oligomer of nucleotides that consists of the sequence selected form the group consisting of Seq ID No. 15, Seq ID No. 17, Seq ID No. 19, and Seq ID No. 18, respectively.

These embodiments are preferred in case, the miRNA mimetics are delivered being packed in lipid based nano particles (LNPs). If LNP particles are used for delivery, the dose might be between 0.01 and 5 mg/kg of the mass of miRNA mimetics per kg of subject to be treated, preferably 0.03 and 3 mg/kg, more preferably 0.1 and 0.4 mg/kg, most preferably 0.3 mg/kg. The administration is of the LNP particles preferably systemic, more preferably intravenous.

The miRNA mimetic can be bound to one or more oligonucleotides that are fully or partially complimentary to the miRNA mimetic and that may or may not form with these oligonucleotides overhang with single stranded regions.

A further embodiment of the invention relates to a pharmaceutical composition as defined herein above wherein the composition is an inhalation composition.

A further embodiment of the invention relates to a pharmaceutical composition as defined herein above wherein the composition is intended for systemic, preferably intravenous administration.

A further embodiment of the invention is a method of treating or preventing of a fibroproliferative disorder, such as IPF or PF-ILD, in a subject in need thereof comprising administering to the subject a pharmaceutical composition as defined above.

For example, the use of a miRNA inhibitor or a miRNA mimetic can be effected by the aerosol route for inhibiting fibrogenesis in the pathological respiratory epithelium in subjects suffering from pulmonary fibrosis and thus restoring the integrity of the pathological tissue so as to restore full functionality.

The viral vector is preferably administered as in an amount corresponding to a dose of virus in the range of 1.0×10¹⁰ to 1.0×10¹⁴ vg/kg (virus genomes per kg body weight), although a range of 1.0×10¹¹ to 1.0×10¹² vg/kg is more preferred, and a range of 5.0×10¹¹ to 5.0×10¹² vg/kg is still more preferred, and a range of 1.0×10¹² to 5.0×10¹¹ is still more preferred. A virus dose of approximately 2.5×10¹² vg/kg is most preferred. The amount of the viral vector to be administered, such as the AAV vector according to the invention, for example, can be adjusted according to the strength of the expression of one or more transgenes.

A further aspect of the invention is the use of viral vectors, miRNA inhibitors and miRNA mimetics according to the invention for combined therapy with either Nintedanib or Pirfenidone.

### USED TERMS AND DEFINITIONS

An expression cassette comprises a transgene and usually a promotor and a polyadenylation signal. The promotor is operably linked to the transgene. A suitable promoter may be selectively or constitutively active in a lung cell, such as an epithelial alveolar cell. Specific non-limiting examples of suitable promoters include constitutively active promoters such as the cytomegalovirus immediate early gene promoter, the Rous sarcoma virus long terminal repeat promoter, the human elongation factor 1a promoter, and the human ubiquitin c promoter. Specific non-limiting examples of lung-specific promoters include the surfactant protein C gene promoter, the surfactant protein B gene promoter, and the Clara cell 10 kD (“CC 10”) promoter.

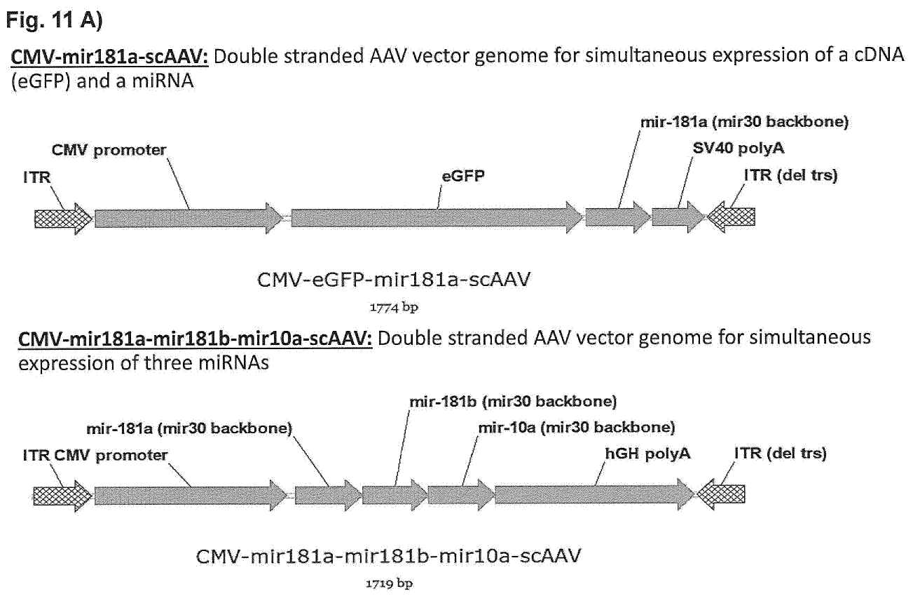

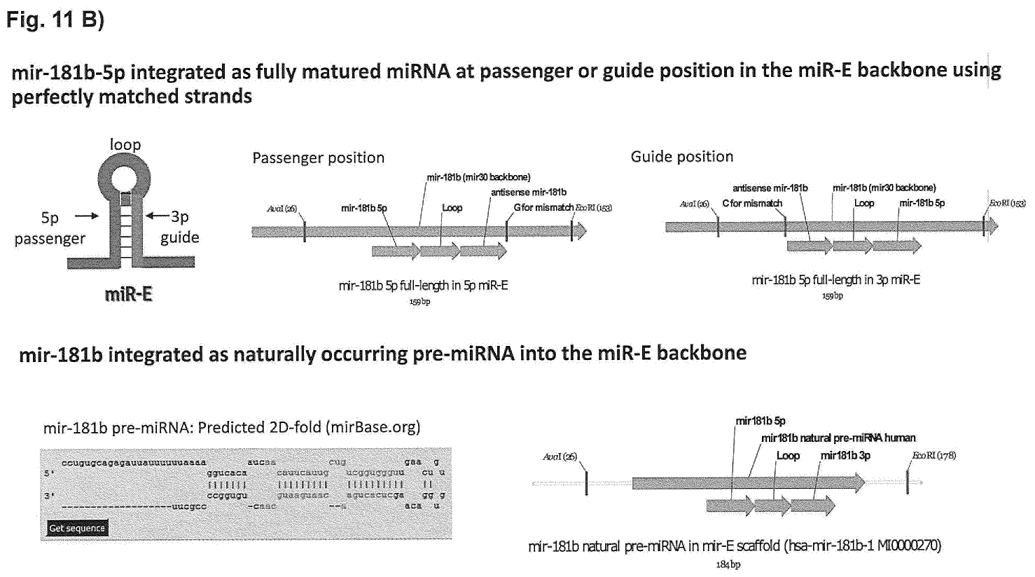

A transgene, depending on the embodiment of the invention, either codes for (i) one or more miRNA e.g. a miRNA having the sequence of Seq ID No. 15 or one or more miRNA that are downregulated in a Bleomycin-induced lung fibrosis model or in an AAV-TGFβ1-induced lung fibrosis model}, or (ii) for an RNA that inhibits the function of one or more miRNA that is upregulated in a Bleomycin-induced lung fibrosis model and in an AAV-TGFβ1-induced lung fibrosis model, or for both alternatives (i) and (ii). The transgene may also contain an open reading frame that encodes for a protein for transduction reporting (such as eGFP, see FIG. 11) or therapeutic purposes.

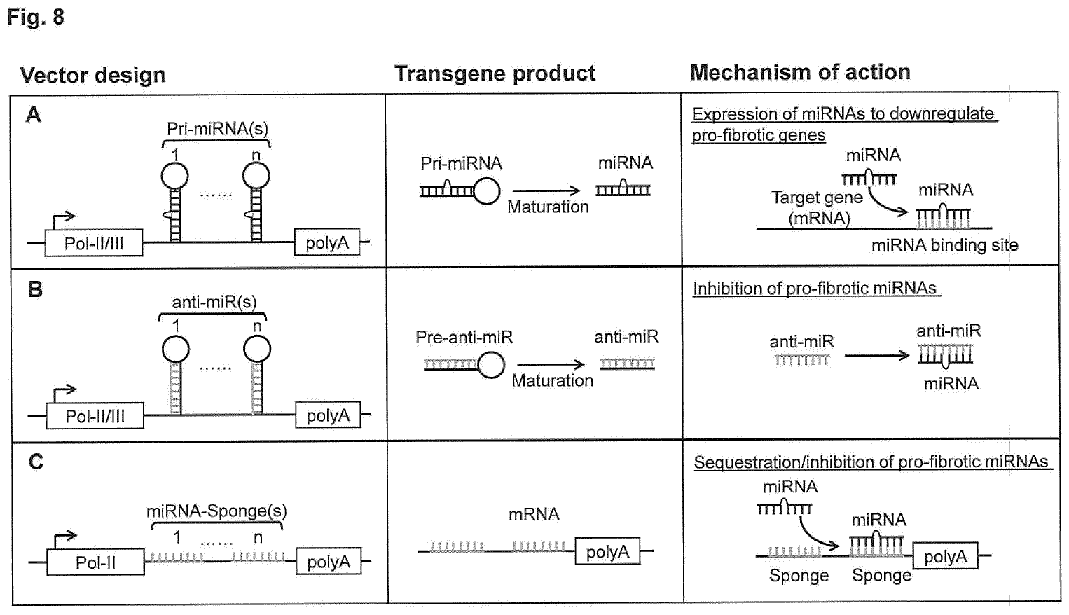

An RNA that inhibits the function of one or more miRNA reduces or abolishes the function of its target miRNA by complementary binding. Two different vector design strategies can be applied, as described in FIGS. 8 B and C:
1). Expression of antisense-like molecules designed to specifically bind to profibrotic miRNAs and thereby inhibit their function (FIG. 8B). Respective molecules, so called anti-miRs, can be incorporated into expression vectors as short hairpin RNAs (shRNAs) or as artificial miRNAs. In analogy to the miRNA supplementation approach, several miRNA-targeting sequences may be combined in a single vector, thereby enabling inhibition of various target miRNAs.
2) Expression of mRNAs containing several copies of miRNA binding sites, so called sponges, aiming to selectively sequester pro-fibrotic miRNAs and thereby inhibit their function (FIG. 8C). For this alternative the inhibiting RNA is not subject to RNAi processing or RNAi maturation.

The term miRNA inhibitor according to the present invention refers to oligomers consisting of a contiguous sequence of 7 to at least 22 nucleotides in length.

The term nucleotide as used herein, refers to a glycoside comprising a sugar moiety (usually ribose or desoxyribose), a base moiety and a covalently linked group (linkage group), such as a phosphate or phosphorothioate internucleotide linkage group. It covers both naturally occurring nucleotides and non-naturally occurring nucleotides comprising modified sugar and/or base moieties, which are also referred to as nucleotide analogues herein. Non-naturally occurring nucleotides include nucleotides which have sugar moieties, such as bicyclic nucleotides or 2′ modified nucleotides or 2′ modified nucleotides such as 2′ substituted nucleotides. Nucleotides with chemical modifications leading to non-naturally occurring nucleotides comprise the following modifications:

(i) Nucleotides which have Non-Natural Sugar Moieties,

Examples are bicyclic nucleotides or 2′ modified nucleotides or 2′ modified nucleotides such as 2′ substituted nucleotides.

(ii) Nucleotides with Phosphorothioate (PS) and Phosphodithioate (PS2) Modifications

To improve serum stability and increase blood concentrations as well as improve nuclease resistance of the miRNAs, a sulfur in one or more nucleotides of the miRNA inhibitor or mimic could exchange an oxygen of the nucleotide phosphate group, which is defined as a phosphorothioate (PS). For some sequences, this could be combined or complemented by a second introduction of a sulfur group to an existing PS, which is defined as a Phosphodithioate PS2. PS2 modifications on distinct positions of the sense strand, like on nucleotide 19+20 or 3+12 (counting from the 5′ end), could further increase serum stability and therefore the pharmacokinetic characteristics of the miRNA inhibitor/miRNA mimetic (ACS Chem. Biol. 2012, 7, 1214-1220).

(iii) Nucleotides with Boranophosphat Modifications

For some miRNA oligonucleotides, it could be beneficial to exchange one oxygen of the ribose phosphate group against a BH3 group. Boranophosphat modifications on one or more nucleotides could increase serum stability, in case the seed region of miRNA oligonucleotides are not modified by other chemical modifications. Boranophosphat modifications could also increase serum stability of miRNA oligonucleotides (Nucleic Acids Research, Vol. 32 No. 20, 5991-6000).

(iv) Nucleotides with 2′O-Methyl Modification

Besides or in addition to phosphate modifications, methylation of the oxygen, bound to the carbon C2 in the ribose ring, could be further options for oligonucleotide modifications. 2′O-methyl ribose modification of the sense strand could increase thermal stability and the resistance to enzymatic digestions.

(v) Nucleotides with 2′OH with Fluorine Modification

It may could also beneficial to modify miRNA oligonucleotides with 2′ OH fluorine modification to enhance serum stability of the oligonucleotide and improve the binding affinity of the miRNA oligonucleotide to its target. 2′ OH fluorine modification, exchanges the hydroxyl group of the carbon C2 in the ribose ring against a fluorine atom. Fluorine modifications could be applied on both strands, sense and anti-sense.

“Nucleotide analogues” are variants of natural oligonucleotides by virtue of modifications in the sugar and/or base moieties. Preferably, without being limited by this explanation, the analogues will have a functional effect on the way in which the oligomer works to bind to its target; for example by producing increased binding affinity to the target and/or increased resistance to nucleases and/or increased ease of transport into the cell. Specific examples of nucleoside analogues are described by Freier and Altman (Nucl. Acid Res., 25: 4429-4443, 1997) and Uhlmann (Curr. Opinion in Drug Development, 3: 293-213, 2000). Incorporation of affinity-enhancing analogues in the oligomer, including Locked Nucleic Acid (LNA™), can allow the size of the specifically binding oligomer to be reduced and may also reduce the upper limit to the size of the oligomer before non-specific or aberrant binding takes place. The term “LNA™” refers to a bicyclic nucleoside analogue, known as “Locked Nucleic Acid” (Rajwanshi et al., Angew Chem. Int. Ed. Engl., 39(9): 1656-1659, 2000). It may refer to an LNA™ monomer, or, when used in the context of an “LNA™ oligonucleotide” to an oligonucleotide containing one or more such bicylic analogues.

Preferably, a miRNA inhibitor of the invention refers to antisense oligonucleotides with sequence complementary to Certain upregulated miRNA (miRNAs selected from the group consisting of the miRNAs of Seq ID Nos. 1, 2, 3, 4, 5, 6, 7, 8, 9, 10, 11, 12, 13, 14, 16, 34, 35 and 36.). These oligomers may comprise or consist of a contiguous nucleotide sequence of a total of 7 to at least 22 contiguous nucleotides in length, up to 70% nucleotide analogues (LNA™). The shortest oligomer (7 nucleotides) will likely correspond to an antisense oligonucleotide with perfect sequence complementarity matching to the first 7 nucleotides located at the 5′ end of mature to Certain up regulated miRNA, and comprising the 7 nucleotide sequence at position 2-8 from 5′ end called the “seed” sequence) involved in miRNA target specificity (Lewis et al., Cell. 2005 Jan. 14; 120(1):15-20).

A Certain upregulated miRNA Target Site Blocker refers to antisense oligonucleotides with sequence complementary to Certain upregulated miRNA binding site located on a specific mRNA. These oligomers may be designed according to the teaching of US 20090137504. These oligomers may comprise or consist of a contiguous nucleotide sequence of a total of 8 to 23 contiguous nucleotides in length. These sequences may span from 20 nucleotides in the 5′ or the 3′ direction from the sequence corresponding to the reverse complement of Certain upregulated miRNA “seed” sequence.

The term miRNA mimetic of the invention is an oligomer capable of specifically increasing the activity of Certain (mainly downregulated) miRNA wherein the term Certain (mainly downregulated) miRNA means a miRNA that has a sequence selected from the group consisting of Seq ID No. 15, 17, 18, 19, 20, 21, 22, 23, 24, 25, 26, 27, 37, 38 and 39, preferably of Seq ID No, 15, 17, 19, 18, and 20, most preferred 15, 17 and 19, even more preferred Seq ID No. 15. The term miRNA mimetic encompasses salts, including pharmaceutical acceptable salts. The miRNA mimetic of a miRNA elevates the concentration of functional equivalents of said miRNA in the cell thereby increasing the overall activity of said miRNA.

miRNA mimetics of miRNA 212-5p, miRNA 181a-5p, miRNA 181b-5p, and miRNA 10a, respectively are intended for use in the treatment of a fibroproliferative disorder, and wherein the miRNA mimetic is an oligomer of nucleotides that consists of the sequence of Seq ID No. 15, of Seq ID No. 17, Seq ID No. 18, and Seq ID No. 19, respectively with proviso (a), (b) and (c), (a) and (c), (a) and (d), or (c) and (d),
(a) the oligomer optionally comprises nucleotides with chemical modifications leading to non-naturally occurring nucleotides that show the basepairing behavior at the corresponding position (AU and GC) as determined by the sequence of the respective miRNA, preferably chemical modifications as set forth under (i) to (v) herein above;
(b) the oligomer optionally comprises nucleotide analogues that show the base-pairing behavior at the corresponding position (AU and GC) as determined by the sequence of the respective miRNA; preferably the nucleotide analogues described by Freier and Altman (Nucl. Acid Res., 25: 4429-4443, 1997) and Uhlmann (Curr. Opinion in Drug Development, 3: 293-213, 2000) or bicyclic analogues described herein above;
(c) the oligomer is optionally lipid conjugated to facilitate drug delivery.

Lipid conjugated oligomers are well known in the art, see Osborne et al. NUCLEIC ACID THERAPEUTICS Volume 28, Number 3, 2018 with references.

Oligomer consisting of the sequence of the corresponding miRNA means that the oligomer comprises the sequence of the corresponding miRNA and has as many covalently attached nucleotide building blocks (optionally with chemical modifications) or nucleotide analogues as said miRNA.

A miRNA mimetic can be bound to one or more oligonucleotides that are fully or partially complimentary to the miRNA mimetic and that may or may not form with these oligonucleotides overhangs with single stranded regions.

It is preferred that the miRNA mimetic has at least 80%, more preferably at least 90%, even more preferably more than 95% of the biologic effect of the same amount of the natural miRNA as determined by one or more experiments as described under Example 1.11.

miRNA mimetics or miRNA inhibitors can also be delivered as naturally- and non-naturally occurring nucleotides, packed in lipid based nano particles (LNPs). The application comprises the delivery with three classes of LNPs: (i) cationic LNPs, (ii) neutral LNPs and (iii) ionizable LNPs. Whereas cationic LNPs are mainly characterized by a high content of 1,2-dioleyl-3-trimethylammonium propane, 1,2-dioleyloxy-N,N-dimethyl-3-aminopropane, dioctadecylamidoglycylspermine, 3-(N—(N0,N0-dimethylaminoethane)carbamoyl) cholesterol and pegylated modifications. Neutral lipids are mainly characterized by phosphatidylcholine, cholesterol and 1,2-dioleoyl-sn-glycero-3-phosphoethanolamines. Ionizable LNPs are mainly characterized by a major content of 1,2-dioleyloxy-N,N-dimethyl-3-aminopropane and 1,2-dioleyl-3-trimethylammonium propane (see, e.g. Sun, S, Molecules 2017, 22, 1724).

If LNP particles are used for delivery, the dose might be between 0.01 and 5 mg/kg of the mass of miRNA mimetics per kg of subject to be treated, preferably 0.03 and 3 mg/kg, more preferably 0.1 and 0.4 mg/kg, most preferably 0.3 mg/kg. The administration of the LNP particles is preferably systemic, more preferably intravenous.

### EXAMPLES

1. Materials and Methods

1.1 AAV Production, Purification and Quantification

HEK-293h cells were cultivated in DMEM+GlutaMAX media supplemented with 10% fetal calf serum. Three days before transfection, the cells were seeded in 15 cm tissue culture plates to reach 70-80% confluency on the day of transfection. For transfection, 0.5 μg total DNA per cm² of culture area were mixed with 1/10 culture volume of 300 mM CaCl₂ as well as all plasmids required for AAV production in an equimolar ratio. The plasmid constructs were as follows: One plasmid encoding the AAV6.2 cap gene (Strobel B et al., 2015); a plasmid harboring an AAV2 ITR-flanked expression cassette containing a CMV promoter driving expression of a codon-usage optimized murine Tgfb1 gene and a hGh poly(A) signal, whereby the Tgfb1 sequence contains C223S and C225S mutations that increase the fraction of active protein (Brunner A M et al., 1989); a pHelper plasmid (AAV Helper-free system, Agilent). For GFP and stuffer control vector production, the Tgfb1 plasmid was exchanged for an eGFP plasmid, harboring an AAV2 ITR-flanked CMV-eGFP-SV40 pA cassette and AAV-stuffer control plasmid, containing an AAV2 ITR-flanked non-coding region derived from the 3′-UTR of the E6-AP ubiquitin-protein ligase UBE3A followed by a SV40 poly(A) signal, respectively.

The plasmid CaCl₂ mix was then added dropwise to an equal volume of 2×HBS buffer (50 mM HEPES, 280 mM NaCl, 1.5 mM Na₂HPO₄), incubated for 2 min at room temperature and added to the cells. After 5-6 h of incubation, the culture medium was replaced by fresh medium. The transfected cells were grown at 37° C. for a total of 72 h. Cells were detached by addition of EDTA to a final concentration of 6.25 mM and pelleted by centrifugation at room temperature and 1000×g for 10 min. The cells were then resuspended in “lysis buffer” (50 mM Tris, 150 mM NaCl, 2 mM MgCl₂, pH 8.5). AAV vectors were purified essentially as previously described (Strobel B et al., 2015): For iodixanol gradient based purification, cells harvested from up to 40 plates were dissolved in 8 mL lysis buffer. Cells were then lysed by three freeze/thaw cycles using liquid nitrogen and a 37° C. water bath. For each initially transfected plate, 100 units Benzonase nuclease (Merck) were added to the mix and incubated for 1 h at 37° C. After pelleting cell debris for 15 min at 2500×g, the supernatant was transferred to a 39 mL Beckman Coulter Quick-Seal tube and an iodixanol (OptiPrep, Sigma Aldrich) step gradient was prepared by layering 8 mL of 15%, 6 mL of 25%, 8 mL of 40% and 5 mL of 58% iodixanol solution diluted in PBS-MK (lx PBS, 1 mM MgCl₂, 2.5 mM KCl) below the cell lysate. NaCl had previously been added to the 15% phase at 1 M final concentration. 1.5 μL of 0.5% phenol red had been added per mL to the 15% and 25% iodixanol solutions and 0.5 μL had been added to the 58% phase to facilitate easier distinguishing of the phase boundaries within the gradient. After centrifugation in a 70Ti rotor for 2 h at 63000 rpm and 18° C., the tube was punctured at the bottom. The first five milliliters (corresponding to the 58% phase) were then discarded, and the following 3.5 mL, containing AAV vector particles, were collected. PBS was added to the AAV fraction to reach a total volume of 15 mL and ultrafiltered/concentrated using Merck Millipore Amicon Ultra-15 centrifugal filter units with a MWCO of 100 kDa. After concentration to ˜1 mL, the retentate was filled up to 15 mL and concentrated again. This process was repeated three times in total. Glycerol was added to the preparation at a final concentration of 10%. After sterile filtration using the Merck Millipore Ultrafree-CL filter tubes, the AAV product was aliquoted and stored at −80° C.

1.2 Mouse Models and Functional Readouts

For reporter gene studies, 9-12 week old female C57Bl/6 or Balb/c mice, purchased from Charles River Laboratories, either received 2.9×10¹⁰ vector genomes (vg) of AAV5-CMV-fLuc or 3×10¹¹ vg of AAV6.2-CMV-GFP, respectively, by intratracheal administration under light anesthesia (3-4% isoflurane). Alternatively, C57Bl/6 mice received 3×10¹¹ vg of AAV2-L1-CMV-GFP by intravenous (i.v.) administration. Two to three weeks after AAV administration (see figure descriptions), reporter readouts were performed. For luciferase imaging, mice received 30 mg/kg luciferin as a substrate via intraperitoneal administration prior to image acquisition. In the case of GFP reporters, either histological fresh-frozen lung sections were prepared and analyzed for direct GFP fluorescence by fluorescence microscopy or formalin-fixed paraffin embedded slices were prepared for GFP IHC analysis (see detailed description further below).

For the fibrosis models, male 9-12 week old C57Bl/6 mice purchased from Charles River Laboratories received intratracheal administration of either 2.5×10¹¹ (vg) of AAV-TGFβ1 or AAV-stuffer, 1 mg/kg Bleomycin or physiological NaCl solution in a volume of 50 μL, which was carried out under light anesthesia. Fibrosis was assessed at day 3, 7, 14, 21 and 28 after AAV/Bleomycin administration. Briefly, to assess lung function, mice were anesthetized by intraperitoneal (i.p.) administration of pentobarbital/xylazine hydrochloride, cannulated intratracheally and treated with pancuronium bromide by intravenous (i.v.) administration. Lung function measurement (i.e. lung compliance, forced vital capacity (FVC)) was then conducted using the Scireq flexiVent FX system. Mice were then euthanized by a pentobarbital overdose, the lung was dissected and weighed prior to flushing with 2×700 μL PBS to obtain BAL fluid for differential BAL immune cell and protein analyses (data not shown). The left lung of each mouse was processed for histological assessment by a histopathologist, whereas the right lung was used for total RNA extraction, as detailed below.

1.3 Histology

For the preparation of histological lung samples, the left lung lobe was mounted to a separation funnel filled with 4% paraformaldehyde (PFA) and inflated under 20 cm water pressure for 20 minutes. The filled lobe was then sealed by ligature of the trachea and immersed in 4% PFA for at least 24 h. Subsequently, PFA-fixed lungs were embedded in paraffin. Using a microtome, 3 μm lung sections were prepared, dried, deparaffinized using xylene and rehydrated in a descending ethanol series (100-70%). Masson's trichrome staining was performed using the Varistain Gemini ES Automated Slide Stainer according to established protocols. For GFP-IHC, enzymatic antigen retrieval was performed and antibodies were diluted at indicated ratios in Bond primary antibody diluent (Leica Biosystems). Slides were stained with the 1:1000 diluted polyclonal Abcam rabbit antis GFP antibody ab290 and appropriate isotype control antibodies, respectively. Slides that had only received antigen retrieval served as an additional negative control. Finally, sections were mounted with Merck Millipore Aquatex medium.

1.4 RNA Preparation

For total lung RNA preparation, the right lung was flash frozen in liquid nitrogen immediately after dissection. Frozen lungs were homogenized in 2 mL precooled Qiagen RLT buffer+1% β-mercaptoethanol using the Peqlab Precellys 24 Dual Homogenizer and 7 mL-ceramic bead tubes. 150 μL homogenate were then mixed with 550 μL QIAzol Lysis Reagent (Qiagen). After addition of 140 μL chloroform (Sigma-Aldrich), the mixture was shaken vigorously for 15 sec and centrifuged for 5 min at 12,000×g and 4° C. 350 μL of the upper aqueous RNA-containing phase were then further purified using the Qiagen miRNeasy 96 Kit according to the manufacturer's instructions. After purification, RNA concentration was determined using a Synergy HT multimode microplate reader and the Take3 module (BioTek Instruments). RNA quality was assessed using the Agilent 2100 Bioanalyzer.

1.5 RNA Sequencing

cDNA libraries were prepared using the Illumina TruSeq RNA Sample Preparation Kit. Briefly, 200 ng of total RNA were subjected to polyA enrichment using oligo-dT-attached magnetic beads. PolyA-containing mRNAs were then fragmented into pieces of approximately 150-160 bp. Following reverse transcription with random primers, the second cDNA strand was synthesized by DNA polymerase I. After an end repair process and the addition of a single adenine base, phospho-thymidine-coupled indexing adapters were coupled to each cDNA, which facilitate sample binding to the sequencing flow cell and further allows for sample identification after multiplexed sequencing. Following purification and PCR enrichment of the cDNAs, the library was diluted to 2 nM and clustered on the flow cell at 9.6 pM, using the Illumina TruSeq SR Cluster Kit v3-cBot-HS and the cBot instrument. Sequencing of 52 bp single reads and seven bases index reads was performed on an Illumina HiSeq 2000 using the Illumina TruSeq SBS Kit v3-HS. Approximately 20 million reads were sequenced per sample.

For miRNA, the Illumina TruSeq Small RNA Library Preparation Kit was used to prepare the cDNA library: As a result of miRNA processing by Dicer, miRNAs contain a free 5′-s phosphate and 3′-hydroxal group, which were used to ligate specific adapters prior to first and second strand cDNA synthesis. By PCR, the cDNAs were then amplified and indexed. Using magnetic Agencourt AMPure XP bead-purification (Beckman Coulter), small RNAs were enriched. The samples were finally clustered at 9.6 pM and sequenced, while being spiked into mRNA sequencing samples.

1.6 Computational Processing and Data Analysis (mRNA-Seq and miRNA-Seq Data Processing)

mRNA-Seq reads were mapped to the mouse reference genome GRCm38.p6 and Ensembl mouse gene annotation version 86 (http://oct2016.archive.ensembl.org) using the STAR aligner v. 2.5.2a (Dobin et al., 2013). Raw sequence read quality was assessed using FastQC v0.11.2, alignment quality metrics were checked using RNASeQC v1.18 (De Luca D. S. et al., 2012). Subsequently, duplicated reads in the RNA-Seq samples were marked using bamUtil v1.0.11 and subsequently duplication rates assessed using the dupRadar Bioconductor package v1.4 (Sayols-Puig, S. et al., 2016). Read count vectors were generated using the feature counts package (Liao Y. et al., 2014). After aggregation to count matrices data were normalized using trimmed mean of M-values (TMM) and voom transformed to generate log(counts per million) (CPM) (Ritchie M. E., 2015). Descriptive analyses such as PCA and hierarchical clustering were carried out to identify possible outliers. Differential expression between treatment and respective controls at each time points were carried out using limma with a significance threshold of p adj≤0.05 and abs(log₂FC)≥0.5. Two samples out of 124 in total were excluded for not passing QC criteria. miRNA-Seq reads were trimmed using the Kraken package v.12-274 (Davis M. P. A. et al., 2013) and subsequently mapped to the mouse reference genome GRCm38.p6 and the miRbase v. 21 mouse miRNA (http://mirbase.org) using the STAR aligner v. 2.5.2a. Raw sequence read quality was assessed using FastQC v0.11.2 (http://www.bioinformatics.babraham.uk/project/fastqc/), trimming size and biotype distribution assessed using inhouse scripts. After aggregation to count matrices data were normalized using trimmed mean of M-values (TMM) and voom transformed to generate log(counts per million) (CPM). Descriptive analyses such as PCA and hierarchical clustering were carried out to identify possible outliers. Differential expression between treatment and respective controls at each time points were carried out using limma with a significance threshold of p adj≤0.05 and abs(log₂FC)≥0.5.

1.7 Integrated Data Analysis (Correlation of Functional Parameters and Expression)

Spearman's rho between the measured values for lung function and lung weight vs. the voom transformed log(CPM) of each miRNA and mRNA across all samples of both models and all time points.

1.8 Determination of Putative miRNA-mRNA Target Pairs

To determine mRNA targets of miRNAs, a stepwise approach has been carried out. First lowly expressed miRNAs and mRNAs were removed from the expression matrix. Subsequently the Spearman's rho was calculated between voom transformed log(CPM) of each miRNA vs. each mRNA across all samples of both models and all time points, using the corAndPvalue function from WGCNA v. 1.60 (Langfelder & Horvath, 2008) The set of correlation based putative miRNA-mRNA pairs is defined as all combinations with a correlation ≤−0.6. To add sequence based prediction of putative miRNA-mRNA pairs, all combinations with predictions in at least two out of five most cited miRNA target prediction algorithms (DIANA, Miranda, PicTar, TargetScan, and miRDB) available in the Bioconductor package miRNAtap v. 1.10.0/miRNAtap.db v. 0.99.10 (Pajak & Simpson, 2016) were taken as sequence based pairs. The final set of miRNA-mRNA pairs is the intersection of anticorrelation based and sequence based interaction pairs, reducing the number of predictions significantly to a high-confidence subset.

1.9 Mouse-Human Conservation of miRNA Sequences

For all murine and human miRNAs from miRBase 21 seed regions (position 2 to 7) were extracted. For all combinations of murine and human miRNAs global alignments between the seed regions and the mature were calculated using the pairwise Alignment function from the Bioconductor Biostrings package (v2.46.0). We applied the Needleman-Wunsch algorithm using an RNA substitution matrix with a match score of 1 and a mismatch score of 0. We assigned two categories to the miRNA candidates—“conserved” for miRNAs with an alignment score of 6 in the seed region for mouse-human pairs of miRNAs with the same name, “non-conserved” for miRNAs with an alignment score <6 in the seed region for mouse-human pairs of miRNAs with the same name. In addition, miRNAs with an alignment score for the alignment of the respective mature sequences above 20 is assigned to the category “mature high similarity”.

1.10 Characterization of miRNAs Based on Gene Set Enrichment of Target Gene Sets

The functional characterization of miRNAs is carried out using the enrichment function on the predicted mRNA targets from the MetabaseR package v. 4.2.3 and the gene set categories “pathway maps”, “pathway map folders”, “process networks”, “metabolic networks”, “toxicity networks”, “disease genes”, “toxic pathologies”, “GO processes”, “GO molecular functions”, “GO localizations”. The enrichment function performs a hypergeometric test on the overlap of the query gene set and the reference sets from Metabase. The data retrieval for the characterization of miRNA target sets was carried out on Metabase on Mar. 12, 2018.

1.11 Functional Characterization of miRNAs in Cellular Assays

miRNAs were characterized regarding their impact on the cellular production of the proinflammatory cytokine IL-6 and the pro-fibrotic processes fibroblast proliferation, fibroblasts-to-myofibroblasts transition (FMT), collagen expression and epithelial-tomesenchymal transition (EMT). Unless stated differently in the Figures or Figure Legends, A549, NHBEC (normal human bronchial epithelial cells) or NHLF (normal human lung fibroblast) cells were transiently transfected with miRNA mimetic at a concentration of 2 nM for single miRNAs or 2+2 nM for miRNA combinations. For the latter condition, 4 nM miRNA controls were used. Twenty-four hours later, TGFβ1 was added to the cells at 5 ng/mL concentration and cells were incubated for 24 h (IL-6, proliferation assays and collagen mRNA expression) or 72 h (collagen protein expression, FMT and EMT assays). For the measurement of gene expression, total RNA was extracted from the cells using the Qiagen RNeasy Plus 96 Kit and reversely transcribed into cDNA using the High-Capacity cDNA Reverse Transcription Kit (Thermo Fisher Scientific). IL-6 gene expression was detected by a Taqman qPCR assay (Hs00174131_m1). IL-6 protein was quantified in the cell supernatant using the MSD V-PLEX Proinflammatory Panel 1 Human kit. To assess cell proliferation, cells were grown in presence of TGFβ1 for 24 h and assayed using a WST-1 proliferation assay kit (Sigma/Roche). FMT was assessed by growing NHLF cells as described above, followed by fixation and fluorescent immuno-staining of Collagen 1a1. Images were taken using an IN Cell Analyzer 2000 high-content cellular imaging system and collagen was quantified and normalized to cell number (identified by DAPI-stained nuclei). EMT assessment relied on the same principle, however, using NHBEC cells and immuno-staining of E-cadherin.

Immunoblots were done according to standard methods using novex gels and according buffers from ThermoFisher and electrophoresis devices from BioRad. All primary antibodies were ordered from Cell Signaling Technology.

All cellular assays were performed with either primary lung epithelial cells or primary lung fibroblasts derived from human patient material. Thus, by the heterogeneity of each individual patient donor, e.g. its genetics, environment, cause of disease/surgery, cell isolation, etc., the derived cell also underlie a certain heterogeneity. Thus, it can happen that there are slight assay-to assay variabilities, which explain a certain standard deviation and different assay windows between equal assay formats. Nevertheless, we used primary cells because they are primary patient material and therefore more relevant for the human disease.

2. Results

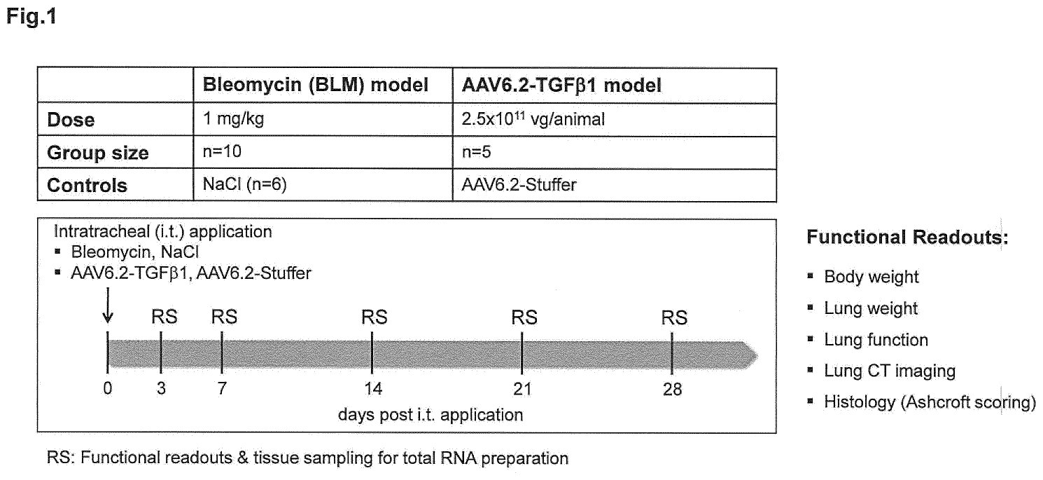

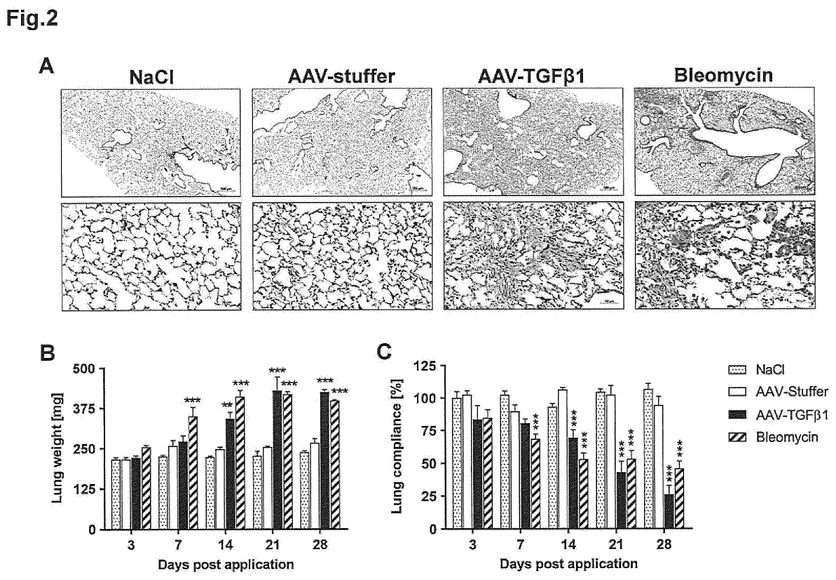

AAV-TGFβ1 and Bleomycin administration induce fibrosing lung pathology in mice. Following administration of either AAV-TGFβ1, Bleomycin or appropriate controls (NaCl, AAV-stuffer), longitudinal fibrosis development was measured over a time period of 4 weeks, as illustrated in FIG. 1. As evident from histological analysis of Massontrichrome stained lung tissue sections on day 21, a pulmonary fibrosis phenotype characterized by thickened alveolar septa, increased extracellular matrix deposition and presence of immune cells was evident in AAV-TGFβ1 and Bleomycin treated animals but absent in NaCl and AAV-stuffer control mice (FIG. 2). A strong increase in lung weight in diseased animals clearly confirmed aberrant ECM deposition and tissue remodeling. Moreover, as a functional consequence, lung function was significantly compromised following TGFβ1 overexpression and Bleomycin treatment, thereby mirroring clinical observations in patients with fibrosing ILDs. Notably, whereas Bleomycin-induced changes in functional readouts occurred about one week prior to the changes in the AAV-TGFβ1 model, a very similar phenotype was evident from day 21.

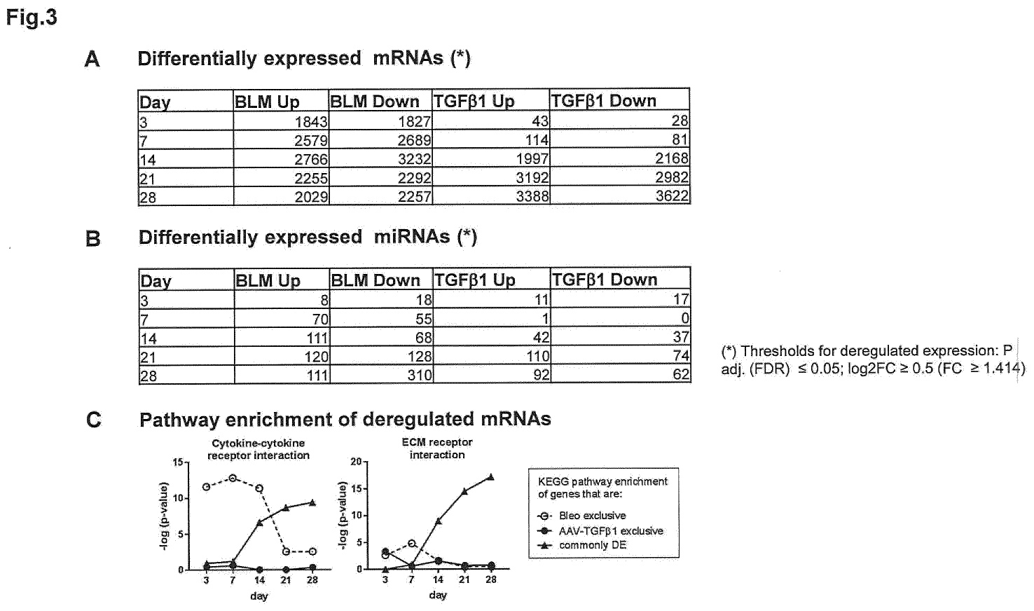

Transcriptional characterization of chronological disease manifestation. In order to dissect the molecular pathways and overall changes in gene expression underlying disease development and progression in the two models of pulmonary fibrosis, RNA was prepared from lung homogenates of each animal and applied to next generation sequencing (NGS) analysis. The number of differentially expressed mRNAs and miRNAs is depicted in FIG. 3. Pathway analysis (FIG. 3C) demonstrated expected enrichment for injury- and acute inflammation related processes at the early time points in the Bleomycin model, whereas inflammation was initially absent in the AAV model and only present during the stages of fibrosis development (day 14 onwards). In contrast, enrichment for remodeling/ECM-associated processes occurred in both disease models in a similar fashion, approximately from day 14 onwards.

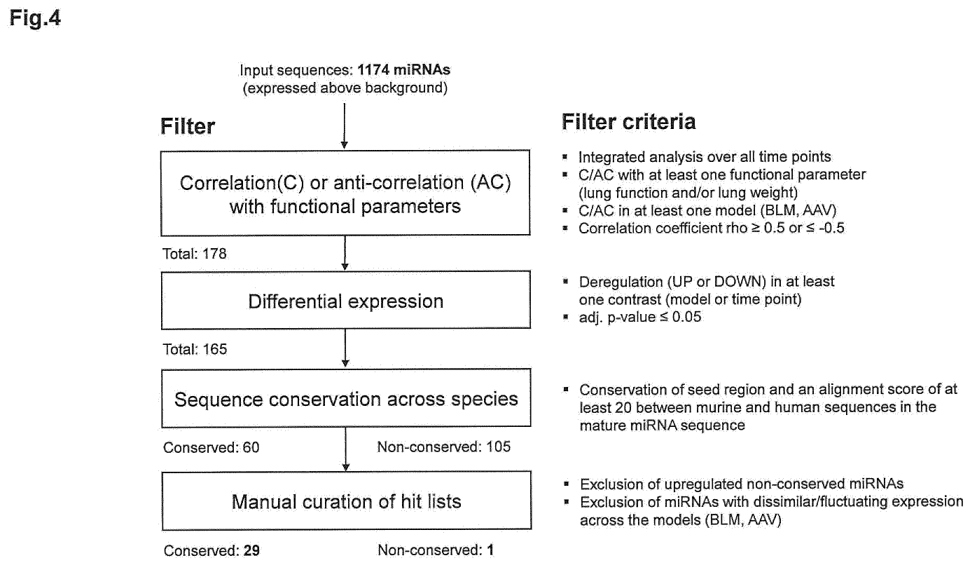

Identification of miRNAs associated with clinically relevant disease phenotypes. To identify candidate miRNAs likely to be directly associated with disease development, a staggered selection strategy using multiple filter criteria was set up (FIG. 4). The central aspect—fibrosis association—was incorporated by selecting only those miRNAs, whose longitudinal expression profiles either strongly correlated or anti-correlated with the observed decrease in lung function or increase in lung weight, respectively. Moreover, a candidate miRNA needed to be differentially expressed at least at one time point in one of the models. miRNAs were then classified according to their species conservation (conserved in humans vs. only present in mice), based on seed sequence and full sequence similarity. The resulting miRNA candidate list was finally hand-curated to dismiss candidates with dissimilar expression in the two disease models and/or fluctuating expression profiles as well as upregulated but non-conserved miRNAs, which could not be targeted in humans. We further eliminated miRNAs that, according to literature text mining results had been previously patented in the context of lung fibrosis. The final hit list is shown in FIG. 5.

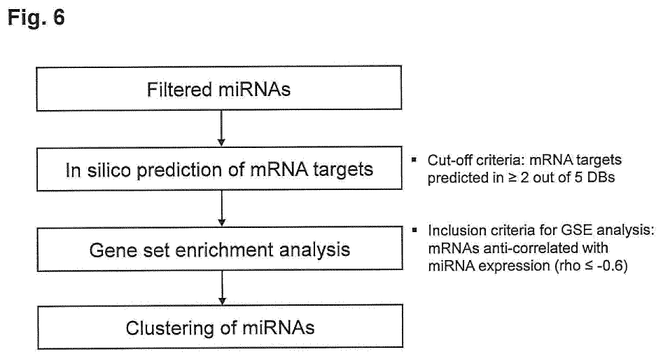

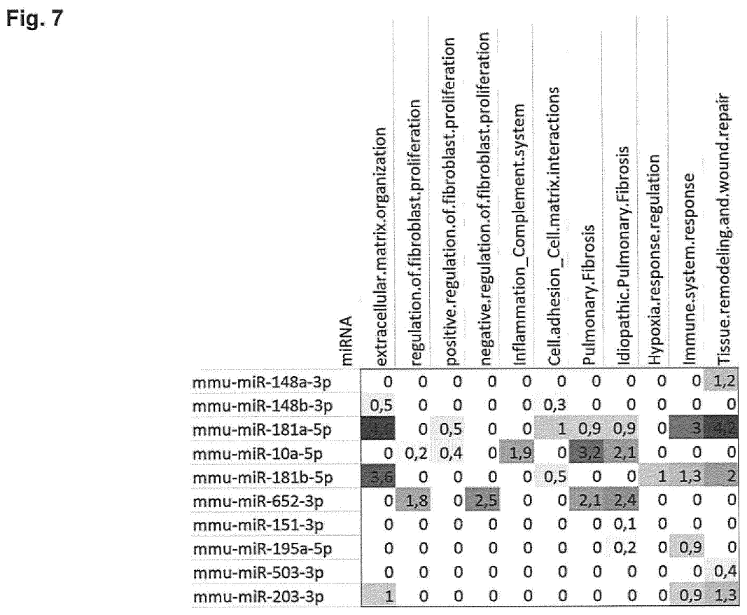

miRNA target prediction (FIG. 6). As an initial approach to characterize the functional role of the miRNAs, putative mRNA targets were predicted computationally, by querying DIANA, MiRanda, PicTar, TargetScan, and miRDB databases via the Bioconductor package miRNAtap (see materials & methods section for details). Targets that were predicted by at least two out of five databases were considered further. Each miRNA target gene set was then analyzed for enrichment of specific disease-relevant processes and FIG. 7 exemplarily illustrates putative functions of genes targeted by specific miRNAs.

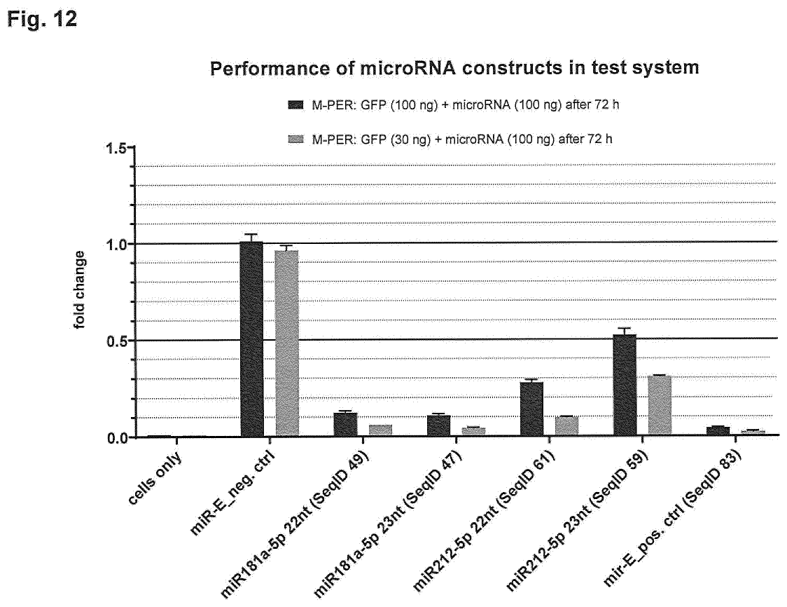

Functionality of miRNAs in mir-E backbone (FIG. 12). A GFP expression construct with target sequences for the miRNAs in the 3′UTR was used to demonstrate the functionality of the miRNA sequences in the mir-E backbone. HEK-293 cells were transiently transfected with the GFP expression construct in combination with a plasmid encoding one of the miRNAs. 72 h after transfection the GFP fluorescence was determined. The fluorescence signal of the negative control, i.e. a miRNA without target sequence in the 3′UTR of the GFP, was set to 100% and the fold change of the fluorescence signals of all other constructs were put into relation to the negative control. The positive control is an optimal mir-E construct and as expected leads to the most pronounced knock-down of GFP. All other construct also lead to a clear knock-down of GFP, indicating that they are not only properly expressed but also correctly processed. The optimal length of the guide strand in the mir-E backbone is 22 nucleotides (nt) which might explain why the miR212-5p with 23 nt is not as efficacious as the one with only 22 nt.

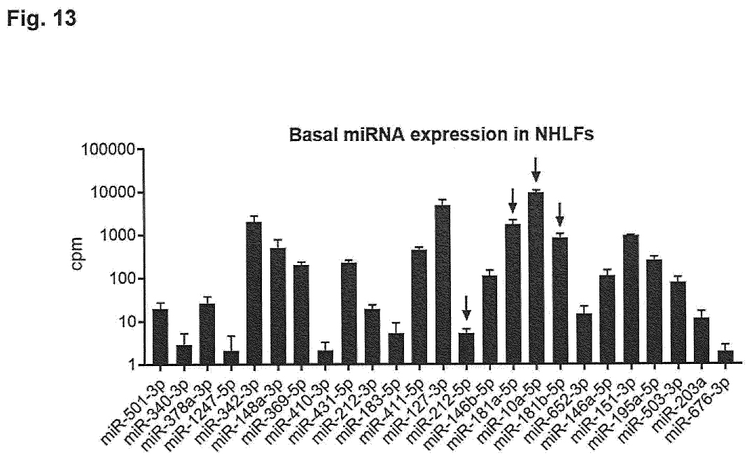

miRNA expression in primary human lung fibroblasts (FIG. 13). To analyze the expression of candidate miRNAs in the human context, small RNA sequencing was performed in primary human lung fibroblasts. As indicated in FIG. 13, robust expression, although at varying levels, was observed for all miRNAs from the candidate list, thereby supporting the concept of species translation of our findings in murine lung fibrosis models to humans.

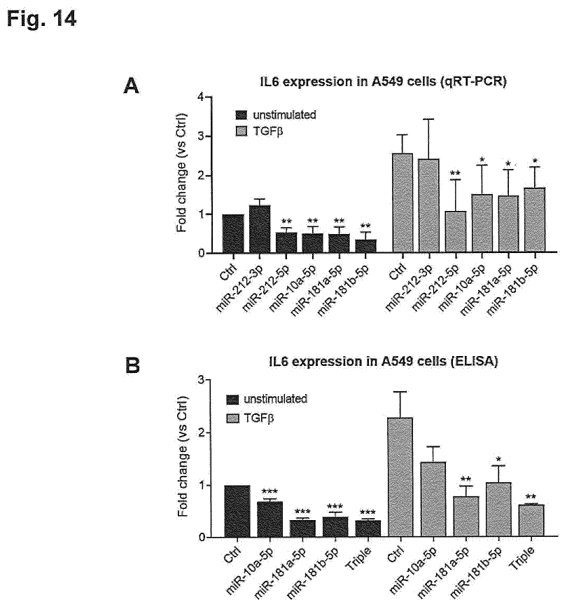

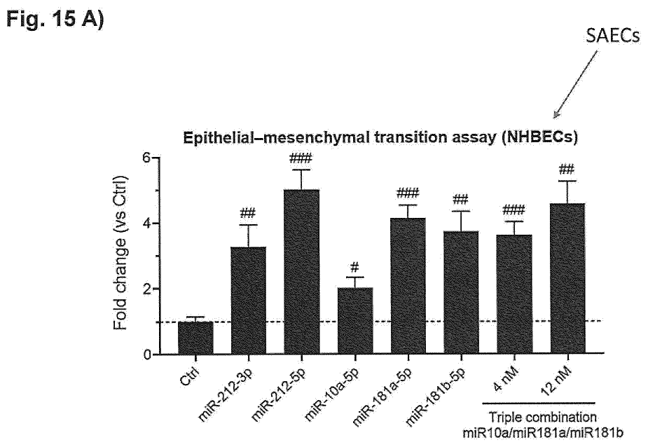

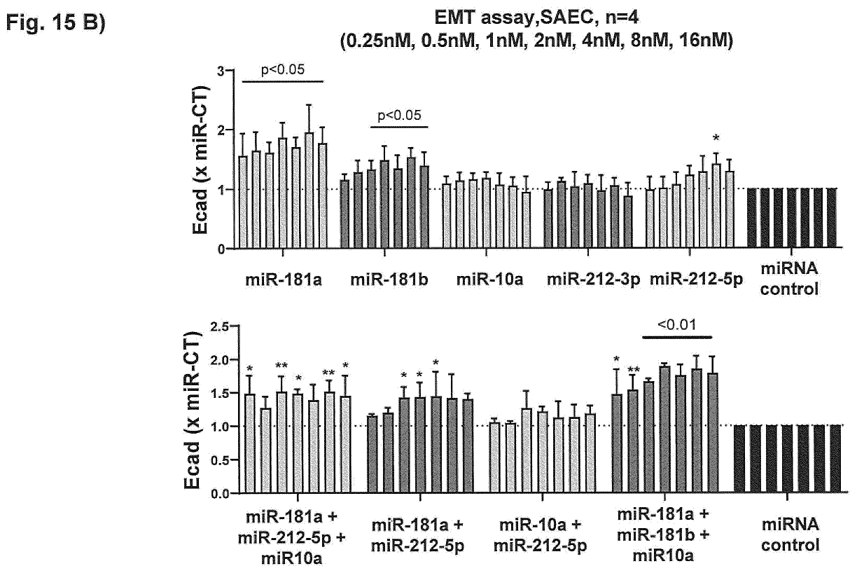

Functional characterization of miRNAs in cellular assays (FIGS. 14-21). To demonstrate anti-fibrotic functions of candidate miRNAs, synthetic miRNA mimetic comprising the fully matured miRNA sequences were generated to perform transient transfection experiments in cellular assays reflecting key mechanisms of fibrotic remodeling. In a first set of experiments the effect of five selected miRNAs (mir-10a-5p, mir-181a-5p, mir-181b-5p, mir-212-3p, mir-212-5p) was analyzed in A549 cells and in primary bronchial airway epithelial cells in the presence or absence of pro-fibrotic TGFβ stimulation. As indicated in FIG. 14 (A), transient transfection of four out of five miRNA mimetic resulted in a significant reduction of TGFβ-induced mRNA expression of IL6, a well described marker gene for inflammation. The only exception was mir-212-3p, which did not show a significant anti-inflammatory effect in this setting. Interestingly, the same result was obtained in unstimulated A549 cells. To further underscore these findings on the protein level, IL6 expression was measured in cell culture supernatants by ELISA. In these experiments mir10a, mir-181a, mir-181b and a triple combination of these miRNAs were investigated. As shown in FIG. 14 (B), all individual miRNAs as well as the triple combination showed significant reduction of IL6 expression in unstimulated and TGFβ-stimulated A549 cells, thereby confirming the anti-inflammatory effect of these miRNAs. Besides its proinflammatory function, TGFβ-also plays a central role as an inducer of epithelial to mesenchymal transition (EMT), a hallmark of fibrotic remodeling in pulmonary fibrosis. During TGFβ-induced EMT, expression of the airway epithelial marker gene E-Cadherin is reduced due to conversion of an epithelial to a fibroblast-like (mesenchymal) cellular phenotype. To assess a potential protective role of selected miRNA candidates on TGFβ-induced EMT, a cellular assay using primary human airway epithelial cells in combination with high-content cellular imaging analysis for quantification of E-Cadherin expression was applied. As depicted in FIG. 15, all miRNAs tested in this setting showed pronounced inhibitory effects on TGFβ-mediated EMT induction, as demonstrated by significantly higher E-Cadherin expression levels in miRNA treated groups as compared to control groups.

Because we also wanted to assess other miRNA combinations, beyond miR-10a+ miR181a-5p+miR-181b-5p, we repeated the former EMT assay, depicted in FIG. 15B. The single miR-181a-5p, miR-181b-5p and miR-212-5p were again able to restore E-cadherin protein expression after TGFβ treatment of lung epithelial cells. Also combinations of miR-181a+miR-212-5p+miR10a and combination of miR-181a+miR-212-5p showed a significant improvement of E-cadherin expression in the EMT assay. Consistently, the best effects were observed with a triple combination of miR-181a-5p+miR-181b-5p+miR10a-5p, which allows a reduction of miRNA dosage to achieve similar effects that miR-181a, miR-181b or miR-10a alone (FIG. 15B). Assay window variabilities between FIG. 15A and FIG. 15B, are explainable by slight assay-to-assay variabilities in combination with different behavior of primary human derived lung epithelial cells from different donors. Nevertheless, the direction of the miRNA effect and its significance stays the same.

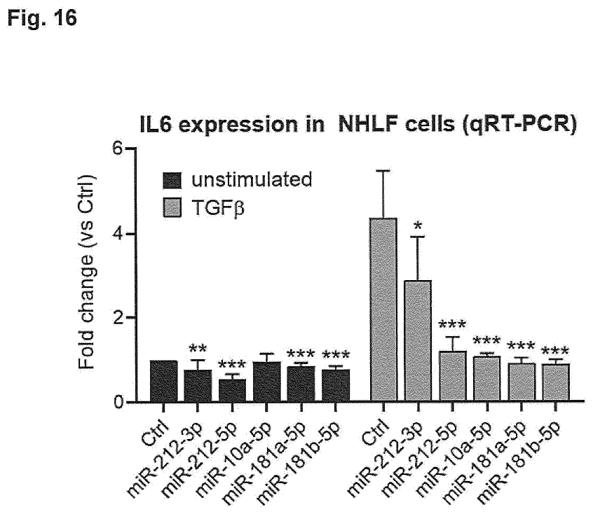

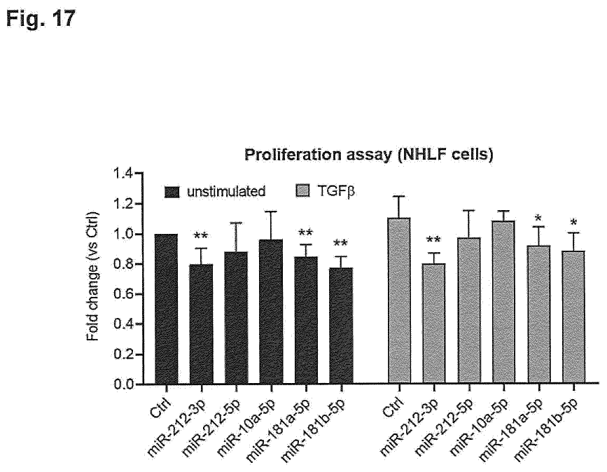

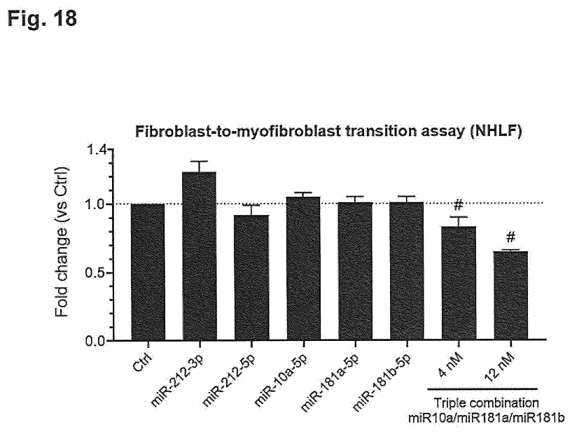

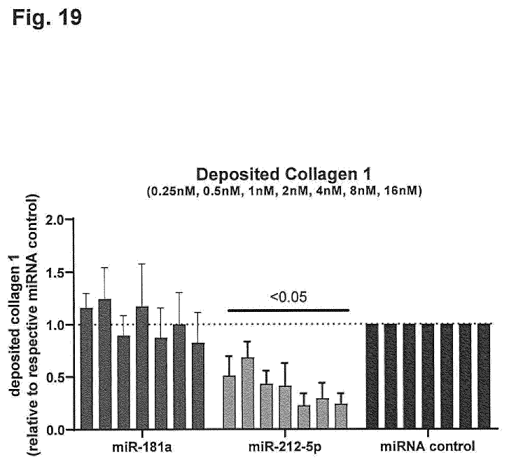

In addition to airway epithelial cells, fibroblasts are considered as a highly relevant cell type for fibrotic processes. By acting as the main source for excessive production of collagen and other extracellular matrix components, fibroblasts directly contribute to lung stiffening associated with impaired lung function and finally loss of structural lung integrity. To further investigate the function of candidate miRNAs during fibroblast activation, transient transfection experiments were carried out in primary human lung fibroblasts under unstimulated and TGFβ-stimulated (pro-fibrotic) conditions. As functional readouts IL6 expression, collagen expression and fibroblast proliferation were assessed in absence or presence of miRNAs. As shown in FIG. 16, all miRNAs analyzed showed significant reduction of IL6 expression in the presence and absence of TGFβ as measured by qRT-PCR. Moreover, mir-212-3p, mir-181a and mir-181b showed inhibitory effects on fibroblast proliferation, both under basal as well as under TGFβ-induced conditions as illustrated in FIG. 17. As depicted in FIG. 18, only the triple combination of mir-10a, mir181a and mir-181b showed a significant and dose-dependent effect on TGFβ-induced FMT compared to control groups, while none of the tested miRNAs showed significant effects when transfected individually. Nevertheless, miR-212-5p showed a trend wise reduction of collagens in this assay (FIG. 18) with this fibroblast donor. To elucidate whether the observed trend wise reduction of collagen deposition by miR212-5p could lead to a significant reduction and because assay variabilities can occur, by working with primary cells, the FMT assay was repeated with 7 different fibroblast donors and a wider range of miRNA dosages (FIG. 19).

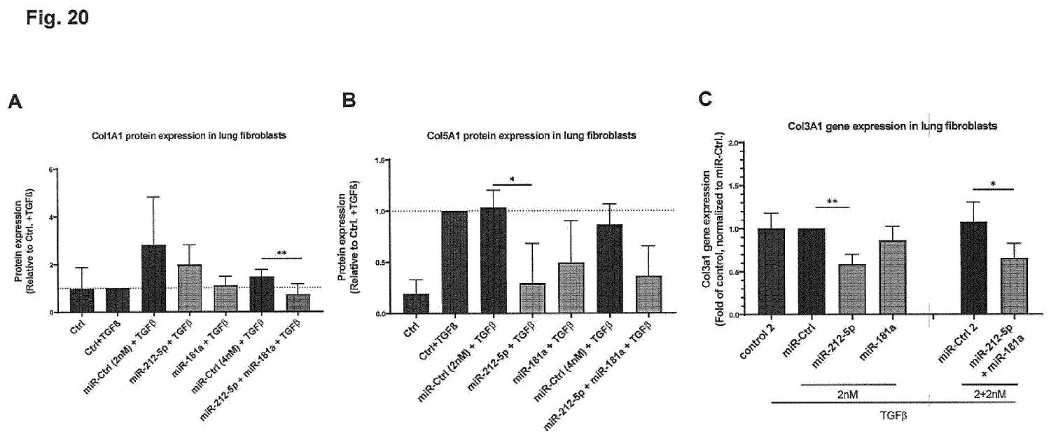

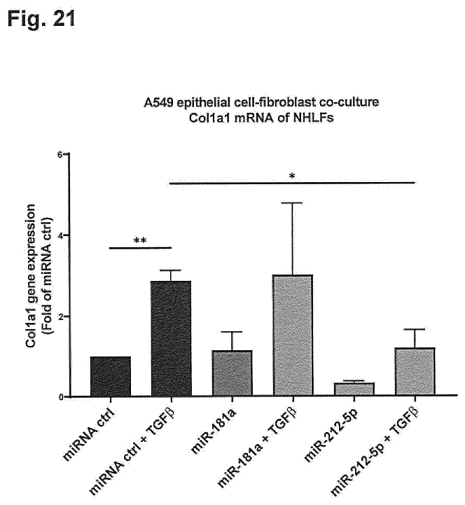

FIG. 19 shows the effect of single miRNA 181a-5p and miR-212-5p on collagen 1 deposition upon TGFβ stimulation in a FMT assay. miR-181a-5p trend wise reduces collagen 1 deposition at higher concentrations. miR-212-5p significantly diminishes collagen 1 deposition of normal and IPF-lung fibroblasts, starting at 0.25 nM, in comparison to the respective miRNA control mimetic (FIG. 19). In addition to collagen 1 deposition, miR-181a5p and miR-212-5p affect also novel collagen expression in human lung fibroblasts beyond collagen 1 (FIGS. 20 and 21). When stimulated with TGFβ, miR-181a-5p and miR-212-5p reduced intracellular collagen 1a1 and collagen 5a1 (FIG. 20A/B). The combination of miR-181a-5p and miR-212-5p showed an additional significant reduction of collagen 1a1 protein expression in comparison to the miRNA negative control (FIG. 20A). In accordance to the reduction of Col1a1 and Col5a1, Col3a1 mRNA expression was also reduced significantly by miR-212-5p and the combination of miR-181a-5p and miR-212-5p (FIG. 20C). To finally validate that the observed anti-fibrotic effects of miR-181a-5p and miR-212-5p on human lung fibroblasts are not (only) mediated via modulation of TGFβ signaling, miRNA mimetic were also tested in an epithelial-fibroblast co-culture, mimicking the cellular fibrotic niche (FIG. 21). In this co-culture system, where pro-fibrotic mediators from epithelial cells activates co-cultured human lung fibroblast, miR-212-5p reduced Col1a1 expression significantly in the human lung fibroblasts, independently of a pre-stimulation of epithelial cells with TGFβ (FIG. 21)

In summary, the functional characterization in human airway epithelial cells and human lung fibroblasts demonstrates anti-inflammatory, anti-proliferative and anti-fibrotic effects for selected miRNA candidates. The most pronounced effects across all assay formats were observed for miR-181a, mir-181b and mir-212-5p, whereas mir-10a and mir-212-3p showed similar profiles although at weaker efficiency compared to the aforementioned miRNAs. In the FMT assay we observed positive effects by miR-10a, miR-181a, miR181b and miR-212-5p, whereas a triple combination of mir-10a, mir-181a and mir-181b showed an improved inhibitory effects in the FMT assay, indicating an additive or synergistic effect for this combination. Overall we observed a very potent anti-fibrotic effect of miR-181a-5p on lung epithelial cells and a very potent anti-fibrotic effect of miR-212-5p on fibroblasts, which suggests that the combination of these two miRNAs are very potent anti-fibrotic combination affecting the two most important cell types in pulmonary fibrosis. Therefore, combinations of miRNA candidates, and especially mimetics of miR-181a-5p and miR-212-5p or its respective mimetics, provide a preferred option for the development of therapeutic approaches with superior efficiency profiles compared to single miRNAs.

Therapeutic applications of miRNAs. To translate the discovery of novel lung-fibrosis associated miRNAs into therapeutic applications, approaches based on vector-mediated expression offer an attractive opportunity for chronic diseases like pulmonary fibrosis by enabling long-lasting expression of miRNAs or miRNA-targeting sequences. As illustrated in FIG. 8, different vector design strategies are available to modulate miRNA function. For supplementation of miRNAs, which are downregulated under fibrotic conditions, vectors using Polymerase-II promoters (e.g. CMV, CBA) or Polymerase-III promoters (e.g. U6, H1) can be applied for the expression of a single miRNA sequence or a combination of several miRNAs (FIG. 8A). While both promoter classes are generally amenable for miRNA expression, Polymerase-II promoter based constructs offer an additional advantage by enabling the use of cell-type-specific promoters thus allowing for the design of more specific and potentially safer vector constructs. Endogenous miRNAs are expressed as precursor molecules, so-called pri-miRNAs, which are first processed via the cellular RNAi machinery into pre-miRNAs and in a second step into the mature and biologically active form. To ensure efficient maturation of vector-derived miRNAs, a sequence of interest can be either expressed as endogenous pre-cursor miRNA or as an artificial miRNA by ems bedding a mature miRNA sequence into a foreign miRNA backbone like e.g. the miR30 scaffold or an optimized version thereof, the so-called miR-E backbone (Fellmann C et al., 2013). In Seq ID No. 40-81 examples for the design of miRNA expression cassettes using the miR-E backbone are provided. While in Seq ID No. 40-69 examples for expression cassettes composed of mature miRNAs or natural pre-miRNAs are described for individual miRNAs, Seq ID No. 70-81 describe combinations of three different miRNAs in a monocistronic expression cassette. All expression cassettes provided, which are embedded in an AAV vector backbone, consist of inverted terminal repeats derived from AAV2, a CMV promoter, a SV40 poly adenylation signal and in some cases the enhanced green fluorescence protein (eGFP) gene upstream of the miRNA sequence(s). To modulate the functionality of miRNAs, which are upregulated under fibrotic conditions, two different vector design strategies can be applied, as described in FIGS. 8 B and C: 1) Expression of antisense-like molecules designed to specifically bind to pro-fibrotic miRNAs and thereby inhibit their function (FIG. 8B). Respective molecules, so called anti-miRs, can be incorporated into expression vectors as short hairpin RNAs (shRNAs) or as artificial miRNAs. In analogy to the miRNA supplementation approach, several miRNA-targeting sequences may be combined in a single vector, thereby enabling inhibition of various target miRNAs. 2) Expression of mRNAs containing several copies of miRNA binding sites, so called sponges, aiming to selectively sequester pro-fibrotic miRNAs and thereby inhibit their function (FIG. 8C). In summary, various vector design strategies are available for functional modulation (supplementation or inhibition) of lung-fibrosis associated miRNAs.

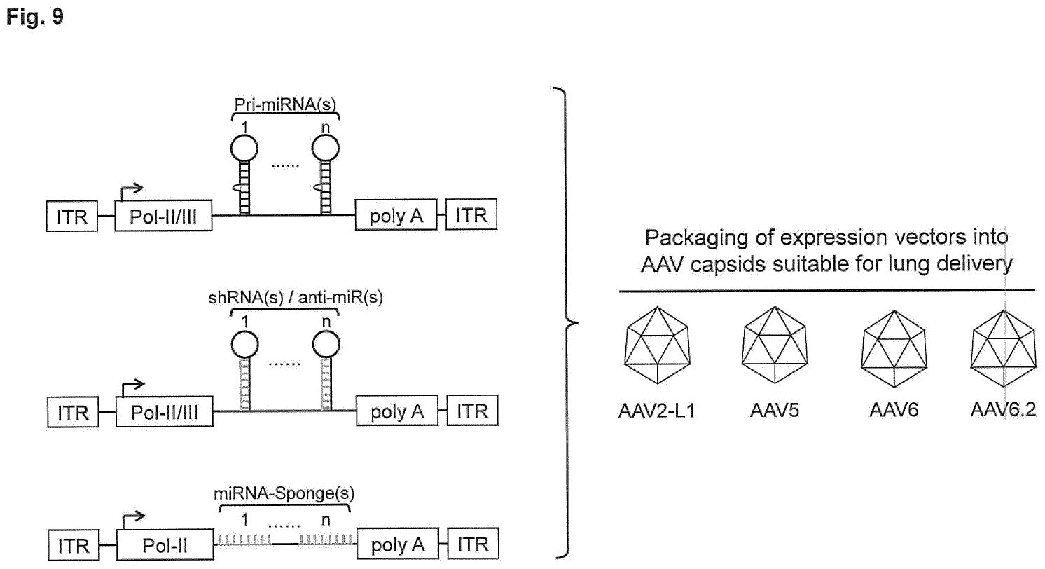

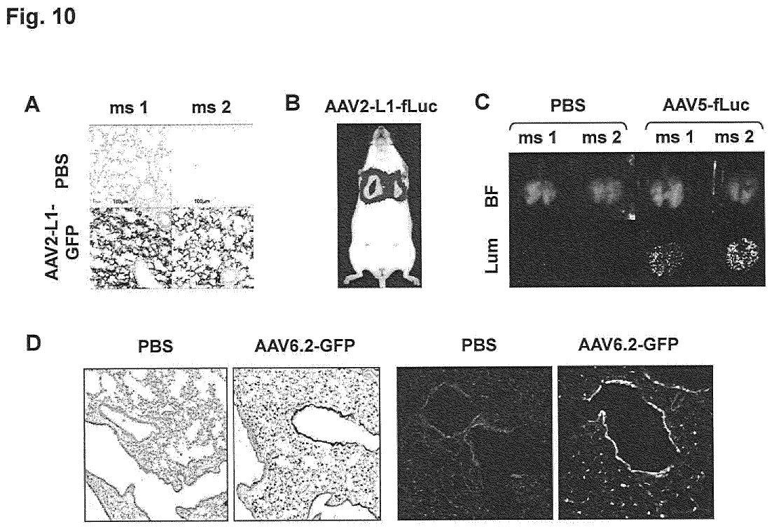

For the delivery of the aforementioned expression constructs to the lung non-viral as well as viral gene therapy vectors can be applied. However, compared to currently available non-viral delivery systems like e.g. liposomes, viral vectors demonstrate superior properties with regard to efficacy and tissue/cell-type selectivity, as demonstrated in various publications over the past years. Moreover, viral vectors offer great potential for engineering approaches to further improve potency, selectivity and safety properties. In recent years, viral vectors based on Adeno-associated virus (AAV) have emerged as one of the most favorable vector systems for in vivo gene therapy based on their excellent pre-clinical and clinical safety profile combined with highly efficient and stable gene delivery to various target organs and cell-types including fully differentiated and non-dividing cells. Since the discovery of the prototypic AAV serotype AAV2 in 1965 (Atchison et al.), various additional serotypes have been isolated from humans, non-human primates and from phylogenetically distinct species such as pigs, birds and others. To date more than 100 natural AAV isolates have been described, which interestingly differ with regard to tissue tropism. By applying capsid engineering approaches the repertoire of available AAV vectors for gene therapy approaches has been further expanded in recent years. Based on a landmark paper by Limberis et al. (2009), in which a systematic comparison of 27 AAV capsid variants and natural serotypes regarding lung transduction is described, AAV5, AAV6 and AAV6.2 were identified as highly suitable capsids for lung delivery following local routes of administration (e.g. intransal or intratracheal instillation). In addition, an engineered AAV capsid variant based on AAV2 (AAV2-L1) has been described recently as a novel vector enabling specific gene delivery to the lung after systemic vector administration (Körbelin et al., 2016). As described in FIG. 9, expression vectors containing miRNA- or miRNA-targeting sequences can be flanked by AAV inverted terminal repeats (ITRs) at the 5′- and the 3′-end, thereby enabling packaging of respective constructs into AAV capsids suitable for lung delivery, as exemplified by AAV2-L1, AAV5, AAV6 and AAV6.2. The potency of AAV-mediated lung delivery using the aforementioned capsid variants was confirmed in mouse studies by using reporter gene expressing constructs (GFP, fLuc) and subsequent assessment of transgene expression by immunohistochemistry (FIG. 10A,D) or in vivo imaging (FIG. 10B,C). On the histological level bronchial airway epithelial cells, alveolar epithelial cells and parenchymal cells were positively stained for reporter gene expression, indicating successful gene delivery to these cell types. Moreover, in the case of systemically delivered AAV2-L1 quantitative transgene expression was additionally detected in lung endothelial cells. Of note, transgene expression was stable with no decline of expression levels up to six months after the initial vector administration (data not shown). In summary AAV vectors represent a highly attractive delivery system for stable expression of therapeutic miRNAs or miRNA-targeting sequences in disease-relevant cell types of the lung thereby offering a novel and highly innovative multi-targeted treatment approach for IPF and other fibrosing interstitial lung diseases with a high unmet medical need.

### LIST OF REFERENCES

Atchison R W, Casto B C, Hammon W M; Adenovirus-associated defective virus particles; Science. 1965 Aug. 13; 149(3685):754-6
Brunner A M, Marquardt H, Malacko A R, Lioubin M N, Purchio A F (1989) Site-directed mutagenesis of cysteine residues in the pro region of the transforming growth factor beta 1 precursor. Expression and characterization of mutant proteins; J Biol Chem 264(23): 13660-13664
Davis M P, van Dongen S, Abreu-Goodger C, Bartonicek N, Enright AJ; Kraken: a set of tools for quality control and analysis of high-throughput sequence data; Methods, 63 (1), (2013), pp. 41-49
DeLuca D S, Levin J Z, Sivachenko A, Fennell T, Nazaire M D, Williams C, Reich M, Winckler W, Getz G; RNA-SeQC: RNA-seq metrics for quality control and process optimization. Bioinformatics. 2012 Jun. 1; 28(11):1530-2
Dobin A, Davis C A, Schlesinger F, Drenkow J, Zaleski C, Jha S, Batut P, Chaisson M, Gingeras T R; STAR: ultrafast universal RNA-seq aligner. Bioinformatics. 2013 Jan. 1; 29(1):15-21
Fellmann C, Hoffmann T, Sridhar V, Hopfgartner B, Muhar M, Roth M, Lai D Y, Barbosa I A, Kwon J S, Guan Y, Sinha N, Zuber J; An Optimized microRNA Backbone for Effective Single-Copy RNAi; Cell Rep. 2013 Dec. 26; 5(6):1704-13
Scott M. Hammond; An overview of microRNAs; Adv Drug Deliv Rev. 2015 Jun. 29; 87: 3-14
Hopkins R B, Burke N, Fell C, Dion G, Kolb M; Epidemiology and survival of idiopathic pulmonary fibrosis from national data in Canada; Eur. Respir. J., 48 (1) (2016), pp. 187-195
Körbelin J, Sieber T, Michelfelder S, Lunding L, Spies E, Hunger A, Alawi M, Rapti K, Indenbirken D, Müller O J, Pasqualini R, Arap W, Kleinschmidt J A, Trepel M; Pulmonary Targeting of Adeno-associated Viral Vectors by Next-generation Sequencingguided Screening of Random Capsid Displayed Peptide Libraries; Mol Ther. 2016 June; 24(6):1050-1061
Langfelder P, Horvath S; WGCNA: an R package for weighted correlation network analysis; BMC Bioinformatics. 2008 Dec. 29; 9:559
Ley B, Collard H R (2013); Epidemiology of idiopathic pulmonary fibrosis; Clin. Epidemiol., 5:483-492
Liao Y, Smyth G K, Shi W; featureCounts: an efficient general purpose program for assigning sequence reads to genomic features; Bioinformatics 30, 923-930 (2014)
Limberis M P, Vandenberghe L H, Zhang L, Pickles R J, Wilson J M; Transduction efficiencies of novel AAV vectors in mouse airway epithelium in vivo and human ciliated airway epithelium in vitro; Mol Ther. 2009 February; 17(2):294-301
Mathai S C, Danoff S K; Management of interstitial lung disease associated with connective tissue disease; BMJ. 2016 Feb. 24; 352
Pajak M and Simpson T I (2016); miRNAtap: miRNAtap: microRNA Targets—Aggregated Predictions. R package version 1.10.0.
Raghu G, Collard H R, Egan J J, et al.; ATS/ERS/JRS/ALAT Committee on Idiopathic Pulmonary Fibrosis An official ATS/ERS/JRS/ALAT statement: idiopathic pulmonary fibrosis: evidence-based guidelines for diagnosis and management; Am J Respir Crit Care Med. 2011; 183(6):788-824
Ritchie M E, Phipson B, Wu D, Hu Y, Law C W, Shi W, Smyth G K; limma powers differential expression analyses for RNA-sequencing and microarray studies; Nucleic Acids Res. 2015 Apr. 20; 43(7)
Sayols S, Scherzinger D, Klein H; dupRadar: a Bioconductor package for the assessment of PCR artifacts in RNA-Seq data; BMC Bioinformatics 17, 428 (2016)
Spagnalo P, Rossi G, Trisolini R, Sverzellati N, Baughman R P, Wells A U. Pulmonary sarcoidosis. Lancet Respir Med. 2018 May; 6(5):389-402.
Strobel B, Duechs M J, Schmid R, Stierstorfer B E, Bucher H, Quast K, Stiller D, Hildebrandt T, Mennerich D, Gantner F, Erb K J, Kreuz S; Modeling Pulmonary Disease Pathways Using Recombinant Adeno-Associated Virus 6.2; Am J Respir Cell Mol Biol. 2015 September; 53(3):291-302.
Strobel B, Miller F D, Rist W, Lamla T. Comparative Analysis of Cesium Chloride- and Iodixanol-Based Purification of Recombinant Adeno-Associated Viral Vectors for Preclinical Applications; Hum Gene Ther Methods. 2015 August; 26(4):147-57
Naso, W F, Tomkowicz, B, Perry W L, Strohl, W Adeno-Associated Virus (AAV) as a Vector for Gene Therapy; BioDrugs (2017) 31:317-334
Wu Z, Asokan A, Grieger J C, Govindasamy L, Agbandje-McKenna M, Samulski RJ Single amino acid changes can influence titer, heparinbinding, and tissue tropism in different adeno-associated virus serotypes; J Virol 2006; 80:11393-11397
Strobel, B. Modeling pulmonary fibrosis by AAV-mediated TGFβ1 Expression: a proof of concept study for AAV-based disease modeling and riboswitch-controlled vector production; Konstanz, Univ., Diss., 2016, 2018, http://kops.unikonstanz.de/handle/123456789/33826
Xianbin, Y., Gene silencing activity of siRNA molecules containing phosphorodithioate substitutions ACS Chem. Biol. 2012, 7, 1214-1220
Hall, A., RNA interference using boranophosphate siRNAs: structure-activity relationships, Nucleic Acids Research, 2004, Vol. 32, No. 20 5991-6000
Freier, S. The ups and downs of nucleic acid duplex stability: structure-stability studies on chemically-modified DNA:RNA duplexes, Nucleic Acids Research (volume 25 issue 22 pages 4429-4443
Uhlmann, Recent advances in the medicinal chemistry of antisense oligonucleotides, Curr Opinion in Drug Development, vol. 3, no. 2, 2000, pages 203-213
Rajwanshi. V, The eight stereoisomers of LNA (locked nucleic acid): a remarkable family of strong RNA binding molecules, Angewandte Chemie, International Edition Volume 39, Issue 9 Pages 1656-1659 Journal 2000
Osborn, M., Improving siRNA Delivery In Vivo Through Lipid Conjugation, Nucleic Acid Therapeutics, Volume 28, Number 3, 2018
Sun, S Enhancing the Therapeutic Delivery of Oligonucleotides by Chemical Modification and Nanoparticle Encapsulation, Molecules 2017, 22, 1724; doi:10.3390/molecules22101724
Beckett. T Inhalation of Nebulized Perfluorochemical Enhances Recombinant Adenovirus and Adeno-Associated Virus-Mediated Gene Expression in Lung Epithelium, Human Gene Therapy Methods 23:98-110 (2012), DOI:10.1089/hgtb.2012.014

TABLE 1
Sequence Seq. ID 40 to 81
In case of divergence with the sequence listing, the table prevails.

>Seq_40_mir-10a-5p 23 nt, miR-E backbone, Passenger position
tcgacttcttaacccaacagaaggctcgagaaggtatattgctgttgacagtgagcgtaccctgtagatccgaatttgtgtagtga
agccacagatgtacacaaattcggatctacagggtctgcctactgcctcggacttcaaggggctagaattcga
>Seq_41_mir-10a-5p 23 nt, miR-E backbone, Guide position
tcgacttcttaacccaacagaaggctcgagaaggtatattgctgttgacagtgagcgaacaaattcggatctacagggtatagt
gaagccacagatgtataccctgtagatccgaatttgtgtgcctactgcctcggacttcaaggggctagaattcga
>Seq_42_mir-10a-5p 22 nt, miR-E backbone, Passenger position
tcgacttcttaacccaacagaaggctcgagaaggtatattgctgttgacagtgagcgtaccctgtagatccgaatttgttagtgaa
gccacagatgtaacaaattcggatctacagggtctgcctactgcctcggacttcaaggggctagaattcga
>Seq 43 mir-10a-5p 22 nt, miR-E backbone, Guide position
tcgacttataacccaacagaaggctcgagaaggtatattgctgttgacagtgagcgccaaattcggatctacagggtatagtg
aagccacagatgtataccctgtagatccgaatttgttgcctactgcctcggacttcaaggggctagaattcga
>Seq 44 mir-10a-5p, natural pre-miRNA in miR-E backbone, Human (hsa-mir-10a
MI0000266)
tcgacttcttaacccaacagaaggctcgagaaggtatattgctgttggatctgtctgtcttctgtatataccctgtagatccgaattt
gtgtaaggaattttgtggtcacaaattcgtatctaggggaatatgtagttgacataaacactccgctctctcggacttcaaggggc
tagaattcga
>Seq 45 mir-10a-5p, natural pre-miRNA in miR-E backbone, Mouse (mmu-mir-10a
MI0000685)
tcgacttcttaacccaacagaaggctcgagaaggtatattgctgttggacctgtctgtcttctgtatataccctgtagatccgaattt
gtgtaaggaattttgtggtcacaaattcgtatctaggggaatatgtagttgacataaacactccgctcactcggacttcaaggggc
tagaattcga
>Seq_46_mir-181a-5p 23 nt, miR-E backbone, Passenger position
tcgacttataacccaacagaaggctcgagaaggtatattgctgttgacagtgagcgaacattcaacgctgtcggtgagttagtg
aagccacagatgtaactcaccgacagcgttgaatgtgtgcctactgcctcggacttcaaggggctagaattcga
>Seq_47_mir-181a-5p 23 nt, miR-E backbone, Guide position
tcgacttataacccaacagaaggctcgagaaggtatattgctgttgacagtgagcgcctcaccgacagcgttgaatgtttagtg
aagccacagatgtaaacattcaacgctgtcggtgagttgcctactgcctcggacttcaaggggctagaattcga
>Seq_48_mir-181a-5p 22 nt, miR-E backbone, Passenger position
tcgacttataacccaacagaaggctcgagaaggtatattgctgttgacagtgagcgaacattcaacgctgtcggtgagtagtg
aagccacagatgtactcaccgacagcgttgaatgtgtgcctactgcctcggacttcaaggggctagaattcga
>Seq_49_mir-181a-5p 22 nt, miR-E backbone, Guide position
tcgacttataacccaacagaaggctcgagaaggtatattgctgttgacagtgagcgatcaccgacagcgttgaatgMagtga
agccacagatgtaaacattcaacgctgtcggtgagtgcctactgcctcggacttcaaggggctagaattcga
>Seq_50_mir-181a-5p, natural pre-miRNA in miR-E backbone, Human (hsa-mir-181a-
1 MI0000289)
tcgacttataacccaacagaaggctcgagaaggtatattgctgttgtgagttttgaggttgcttcagtgaacattcaacgctgtcg
gtgagtttggaattaaaatcaaaaccatcgaccgttgattgtaccctatggctaaccatcatctactccactcggacttcaagggg
ctagaattcga
>Seq_51_mir-181a-5p, natural pre-miRNA in miR-E backbone, Mouse (mmu-mir-
181a-1 MI0000697)
tcgacttcttaacccaacagaaggctcgagaaggtatattgctgttgggttgcttcagtgaacattcaacgctgtcggtgagtttg
gaattcaaataaaaaccatcgaccgttgattgtaccctatagctaaccctcggacttcaaggggctagaattcga
>Seq_52_mir-181b-5p 23 nt, miR-E backbone, Passenger position
tcgacttataacccaacagaaggctcgagaaggtatattgctgttgacagtgagcgaacattcattgctgtcggtgggttagtg
aagccacagatgtaacccaccgacagcaatgaatgtgtgcctactgcctcggacttcaaggggctagaattcga
>Seq_53_mir-181b-5p 23 nt, miR-E backbone, Guide position
tcgacttcttaacccaacagaaggctcgagaaggtatattgctgttgacagtgagcgccccaccgacagcaatgaatgtttagt
gaagccacagatgtaaacattcattgctgtcggtgggttgcctactgcctcggacttcaaggggctagaattcga
>Seq_54_mir-181b-5p 22 nt, miR-E backbone, Passenger position
tcgacttataacccaacagaaggctcgagaaggtatattgctgttgacagtgagcgaacattcattgctgtcggtgggtagtga
agccacagatgtacccaccgacagcaatgaatgtgtgcctactgcctcggacttcaaggggctagaattcga
>Seq_55_mir-181b-5p 22 nt, miR-E backbone, Guide position
tcgacttcttaacccaacagaaggctcgagaaggtatattgctgttgacagtgagcgaccaccgacagcaatgaatgtttagtg
aagccacagatgtaaacattcattgctgtcggtgggtgcctactgcctcggacttcaaggggctagaattcga
>Seq_56_mir-181b-5p, natural pre-miRNA in miR-E backbone, Human (hsa-mir-181b-
1 M10000270)
tcgacttataacccaacagaaggctcgagaaggtatattgctgttgcctgtgcagagattattttttaaaaggtcacaatcaacat
tcattgctgtcggtgggttgaactgtgtggacaagctcactgaacaatgaatgcaactgtggccccgcttctcggacttcaagg
ggctagaattcga
>Seq_57_mir-181b-5p, natural pre-miRNA in miR-E backbone, Mouse (mmu-mir-
181b-1 MI0000723)
tcgacttataacccaacagaaggctcgagaaggtatattgctgttgaggtcacaatcaacattcattgctgtcggtgggttgaac
tgtgtagaaaagctcactgaacaatgaatgcaactgtggccctcggacttcaaggggctagaattcga
>Seq_58_mir-212-5p 23 nt, miR-E backbone, Passenger position
tcgacttcttaacccaacagaaggctcgagaaggtatattgctgttgacagtgagcgaccttggctctagactgcttacttagtga
agccacagatgtaagtaagcagtctagagccaaggctgcctactgcctcggacttcaaggggctagaattcga
>Seq_59_mir-212-5p 23 nt, miR-E backbone, Guide position
tcgacttcttaacccaacagaaggctcgagaaggtatattgctgttgacagtgagcgcgtaagcagtctagagccaaggttagt
gaagccacagatgtaaccttggctctagactgcttacttgcctactgcctcggacttcaaggggctagaattcga
>Seq_60_mir-212-5p 22 nt, miR-E backbone, Passenger position
tcgacttcttaacccaacagaaggctcgagaaggtatattgctgttgacagtgagcgaccttggctctagactgcttactagtga
agccacagatgtagtaagcagtctagagccaaggctgcctactgcctcggacttcaaggggctagaattcga
>Seq_61_mir-212-5p 22 nt, miR-E backbone, Guide position
tcgacttcttaacccaacagaaggctcgagaaggtatattgctgttgacagtgagcgataagcagtctagagccaaggttagtg
aagccacagatgtaaccttggctctagactgcttactgcctactgcctcggacttcaaggggctagaattcga
>Seq_62_mir-212-5p, natural pre-miRNA in miR-E backbone, Human (hsa-mir-212
MI0000288)
tcgacttataacccaacagaaggctcgagaaggtatattgctgttgcggggcaccccgcccggacagcgcgccggcacctt
ggctctagactgcttactgcccgggccgccctcagtaacagtctccagtcacggccaccgacgcctggccccgccctcggac
ttcaaggggctagaattcga
>Seq_63_mir-212-5p, natural pre-miRNA in miR-E backbone, Mouse (mmu-mir-212
MI0000696)
tcgacttcttaacccaacagaaggctcgagaaggtatattgctgttggggcagcgcgccggcaccttggctctagactgcttac
tgcccgggccgccttcagtaacagtctccagtcacggccaccgacgcctggcccctcggacttcaaggggctagaattcga
>Seq_64_scAAV-CMV-eGFP-mir181b-5p (23 nt in miR-E backbone)-SV40pA, Passenger
position
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgatcagcagataccccgaccatatgaagcagcacgacttcttcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttgacagtgagcgaacattcattgctgtcggtgggttagtgaagccacagatgtaacccaccgac
agcaatgaatgtgtgcctactgcctcggacttcaaggggctagaattcgagacttgtttattgcagcttataatggttacaaataaa
gcaatagcatcacaaatttcacaaataaagcatttttttcactgcattctagttgtggtttgtccaaactcatcaatgtatcttaacgc
ggccgagatctccactccctctctgcgcgctcgctcgctcactgaggccgggcgaccaaaggtcgcccgacgcccgggcttt
gcccgggcggcctcagtgagcgagcgagcgcgcagctgcctgcagg
>Seq_65_scAAV-CMV-eGFP-mir181b-5p (23 nt in miR-E backbone)-SV40pA, Guide
position
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgatcagcagataccccgaccatatgaagcagcacgacttatcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttgacagtgagcgccccaccgacagcaatgaatgtttagtgaagccacagatgtaaacattcattg
ctgtcggtgggttgcctactgcctcggacttcaaggggctagaattcgagacttgtttattgcagcttataatggttacaaataaag
caatagcatcacaaatttcacaaataaagcatttttttcactgcattctagttgtggtttgtccaaactcatcaatgtatcttaacgcg
gccgagatctccactccctctctgcgcgctcgctcgctcactgaggccgggcgaccaaaggtcgcccgacgcccgggctttg
cccgggcggcctcagtgagcgagcgagcgcgcagctgcctgcagg
>Seq_66_scAAV-CMV-eGFP-mir181b-5p (22 nt in miR-E backbone)-SV40pA, Passenger
position
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgcttcagcagataccccgaccatatgaagcagcacgacttcttcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttgacagtgagcgaacattcattgctgtcggtgggtagtgaagccacagatgtacccaccgacag
caatgaatgtgtgcctactgcctcggacttcaaggggctagaattcgagacttgtttattgcagcttataatggttacaaataaagc
aatagcatcacaaatttcacaaataaagcatttttttcactgcattctagttgtggtttgtccaaactcatcaatgtatcttaacgcgg
ccgagatctccactccctctctgcgcgctcgctcgctcactgaggccgggcgaccaaaggtcgcccgacgcccgggcMgc
ccgggcggcctcagtgagcgagcgagcgcgcagctgcctgcagg
>Seq_67_scAAV-CMV-eGFP-mir181b-5p (22 nt in miR-E backbone)-SV40pA, Guide
position
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgatcagcagataccccgaccatatgaagcagcacgacttatcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttgacagtgagcgaccaccgacagcaatgaatgtttagtgaagccacagatgtaaacattcattgc
tgtcggtgggtgcctactgcctcggacttcaaggggctagaattcgagacttgtttattgcagcttataatggttacaaataaagc
aatagcatcacaaatttcacaaataaagcatttttttcactgcattctagttgtggtttgtccaaactcatcaatgtatcttaacgcgg
ccgagatctccactccctctctgcgcgctcgctcgctcactgaggccgggcgaccaaaggtcgcccgacgcccgggcMgc
ccgggcggcctcagtgagcgagcgagcgcgcagctgcctgcagg
>Seq_68_scAAV-CMV-eGFP-mir181b-5p (natural pre-miRNA, human)-SV40pA
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgacccatggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgatcagcagataccccgaccatatgaagcagcacgacttatcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttgcctgtgcagagattattttttaaaaggtcacaatcaacattcattgctgtcggtgggttgaactgt
gtggacaagctcactgaacaatgaatgcaactgtggccccgcttctcggacttcaaggggctagaattcgagacttgtttattgc
agatataatggttacaaataaagcaatagcatcacaaatttcacaaataaagcatttttttcactgcattctagttgtggtttgtcca
aactcatcaatgtatcttaacgcggccgagatctccactccctctctgcgcgctcgctcgctcactgaggccgggcgaccaaa
ggtcgcccgacgcccgggcMgcccgggcggcctcagtgagcgagcgagcgcgcagctgcctgcagg
>Seq_69_scAAV-CMV-eGFP-mir181b-5p (natural pre-miRNA, mouse)-SV40pA
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgatcagcagataccccgaccatatgaagcagcacgacttatcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttgaggtcacaatcaacattcattgctgtcggtgggttgaactgtgtagaaaagctcactgaacaat
gaatgcaactgtggccctcggacttcaaggggctagaattcgagacttgtttattgcagcttataatggttacaaataaagcaata
gcatcacaaatttcacaaataaagcatttttttcactgcattctagttgtggtttgtccaaactcatcaatgtatcttaacgcggccga
gatctccactccctctctgcgcgctcgctcgctcactgaggccgggcgaccaaaggtcgcccgacgcccgggctttgcccgg
gcggcctcagtgagcgagcgagcgcgcagctgcctgcagg
>Seq_70_scAAV-CMV-eGFP-mir-181a-mir181b-mir10a (all 23 nt in miR-E backbone),
Passenger position
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgatcagcagataccccgaccatatgaagcagcacgacttatcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttgacagtgagcgaacattcaacgctgtcggtgagttagtgaagccacagatgtaactcaccgac
agcgttgaatgtgtgcctactgcctcggacttcaaggggctagaattcgatcgacttcttaacccaacagaaggctcgagaagg
tatattgctgttgacagtgagcgaacattcattgctgtcggtgggttagtgaagccacagatgtaacccaccgacagcaatgaat
gtgtgcctactgcctcggacttcaaggggctagaattcgatcgacttcttaacccaacagaaggctcgagaaggtatattgctgt
tgacagtgagcgtaccctgtagatccgaatttgtgtagtgaagccacagatgtacacaaattcggatctacagggtctgcctact
gcctcggacttcaaggggctagaattcgagacttgtttattgcagcttataatggttacaaataaagcaatagcatcacaaatttca
caaataaagcatttttttcactgcattctagttgtggtttgtccaaactcatcaatgtatcttaacgcggccgagatctccactccctc
tctgcgcgctcgctcgctcactgaggccgggcgaccaaaggtcgcccgacgcccgggctttgcccgggcggcctcagtga
gcgagcgagcgcgcagctgcctgcagg
>Seq_71_scAAV-CMV-eGFP-mir-181a-mir181b-mir10a (all 23 nt in miR-E backbone),
Guide position
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgcttcagcagataccccgaccatatgaagcagcacgacttcttcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttgacagtgagcgcctcaccgacagcgttgaatgtttagtgaagccacagatgtaaacattcaac
gctgtcggtgagttgcctactgcctcggacttcaaggggctagaattcgatcgacttcttaacccaacagaaggctcgagaag
gtatattgctgttgacagtgagcgccccaccgacagcaatgaatgtttagtgaagccacagatgtaaacattcattgctgtcggt
gggttgc ctactgc ctcggacttcaaggggctagaattcgatcgacttcttaacccaacagaaggctcgagaaggtatattgct
gttgacagtgagcgaacaaattcggatctacagggtatagtgaagccacagatgtataccctgtagatccgaatttgtgtgccta
ctgcctcggacttcaaggggctagaattcgagacttgtttattgcagcttataatggttacaaataaagcaatagcatcacaaattt
cacaaataaagcatttttttcactgcattctagttgtggtttgtccaaactcatcaatgtatcttaacgcggccgagatctccactccc
tctctgcgcgctcgctcgctcactgaggccgggcgaccaaaggtcgcccgacgcccgggctttgcccgggcggcctcagtg
agcgagcgagcgcgcagctgcctgcagg
>Seq_72_scAAV-CMV-eGFP-mir-181a-mir181b-mir10a (all 22 nt in miR-E backbone),
Passenger position
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgatcagcagataccccgaccatatgaagcagcacgacttatcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttgacagtgagcgaacattcaacgctgtcggtgagtagtgaagccacagatgtactcaccgaca
gcgttgaatgtgtgcctactgcctcggacttcaaggggctagaattcgatcgacttcttaacccaacagaaggctcgagaaggt
atattgctgttgacagtgagcgaacattcattgctgtcggtgggtagtgaagccacagatgtacccaccgacagcaatgaatgt
gtgcctactgcctcggacttcaaggggctagaattcgatcgacttcttaacccaacagaaggctcgagaaggtatattgctgttg
acagtgagcgtaccctgtagatccgaatttgttagtgaagccacagatgtaacaaattcggatctacagggtctgcctactgcct
cggacttcaaggggctagaattcgagacttgtttattgcagcttataatggttacaaataaagcaatagcatcacaaatttcacaa
ataaagcatttttttcactgcattctagttgtggtttgtccaaactcatcaatgtatcttaacgcggccgagatctccactccctctctg
cgcgctcgctcgctcactgaggccgggcgaccaaaggtcgcccgacgcccgggctttgcccgggcggcctcagtgagcg
agcgagcgcgcagctgcctgcagg
>Seq_73_scAAV-CMV-eGFP-mir-181a-mir181b-mir10a (all 22 nt in miR-E backbone),
Guide position
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgatcagcagataccccgaccatatgaagcagcacgacttatcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttgacagtgagcgatcaccgacagcgttgaatgtttagtgaagccacagatgtaaacattcaacg
ctgtcggtgagtgcctactgcctcggacttcaaggggctagaattcgatcgacttcttaacccaacagaaggctcgagaaggta
tattgctgttgacagtgagcgaccaccgac agcaatgaatgtttagtg aagccacagatgtaaacattcattgctgtcggtgggt
gcctactgcctcggacttcaaggggctagaattcgatcgacttcttaacccaacagaaggctcgagaaggtatattgctgttgac
agtgagcgccaaattcggatctacagggtatagtgaagccacagatgtataccctgtagatccgaatttgttgcctactgcctcg
gacttcaaggggctagaattcgagacttgtttattgcagcttataatggttacaaataaagcaatagcatcacaaatttcacaaata
aagcatttttttcactgcattctagttgtggtttgtccaaactcatcaatgtatcttaacgcggccgagatctccactccctctctgcg
cgctcgctcgctcactgaggccgggcgaccaaaggtcgcccgacgcccgggctttgcccgggcggcctcagtgagcgag
cgagcgcgcagctgcctgcagg
>Seq_74_scAAV-CMV-eGFP-mir-212-5p-mir181b-mir10a (all 23 nt in miR-E backbone),
Passenger position
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgatcagcagataccccgaccatatgaagcagcacgacttatcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttgacagtgagcgaccttggctctagactgcttacttagtgaagccacagatgtaagtaagcagtc
tagagccaaggctgcctactgcctcggacttcaaggggctagaattcgatcgacttcttaacccaacagaaggctcgagaagg
tatattgctgttgacagtgagcgaacattcattgctgtcggtgggttagtgaagccacagatgtaacccaccgacagcaatgaat
gtgtgcctactgcctcggacttcaaggggctagaattcgatcgacttcttaacccaacagaaggctcgagaaggtatattgctgt
tgacagtgagcgtaccctgtagatccgaatttgtgtagtgaagccacagatgtacacaaattcggatctacagggtctgcctact
gcctcggacttcaaggggctagaattcgagacttgtttattgcagatataatggttacaaataaagcaatagcatcacaaatttca
caaataaagcatttttttcactgcattctagttgtggtttgtccaaactcatcaatgtatcttaacgcggccgagatctccactccctc
tctgcgcgctcgctcgctcactgaggccgggcgaccaaaggtcgcccgacgcccgggctttgcccgggcggcctcagtga
gcgagcgagcgcgcagctgcctgcagg
>Seq_75_scAAV-CMV-eGFP-mir-212-5p-mir181b-mir10a (all 23 nt in miR-E backbone),
Guide position
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgcttcagcagataccccgaccatatgaagcagcacgacttcttcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttgacagtgagcgcgtaagcagtctagagccaaggttagtgaagccacagatgtaaccttggctc
tagactgatacttgcctactgcctcggacttcaaggggctagaattcgatcgacttcttaacccaacagaaggctcgagaaggt
atattgctgttgacagtgagcgccccaccgacagcaatgaatgtttagtgaagccacagatgtaaacattcattgctgtcggtgg
gttgcctactgcctcggacttcaaggggctagaattcgatcgacttcttaacccaacagaaggctcgagaaggtatattgctgtt
gacagtgagcgaacaaattcggatctacagggtatagtgaagccacagatgtataccctgtagatccgaatttgtgtgcctact
gcctcggacttcaaggggctagaattcgagacttgtttattgcagatataatggttacaaataaagcaatagcatcacaaatttca
caaataaagcatttttttcactgcattctagttgtggtttgtccaaactcatcaatgtatcttaacgcggccgagatctccactccctc
tctgcgcgctcgctcgctcactgaggccgggcgaccaaaggtcgcccgacgcccgggctttgcccgggcggcctcagtga
gcgagcgagcgcgcagctgcctgcagg
>Seq_76_scAAV-CMV-eGFP-mir-212-5p-mir181b-mir10a (all 22 nt in miR-E backbone),
Passenger position
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgatcagcagataccccgaccatatgaagcagcacgacttatcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttgacagtgagcgaccttggctctagactgcttactagtgaagccacagatgtagtaagcagtcta
gagccaaggctgcctactgcctcggacttcaaggggctagaattcgatcgacttcttaacccaacagaaggctcgagaaggta
tattgctgttgacagtgagcgaacattcattgctgtcggtgggtagtgaagccacagatgtacccaccgacagcaatgaatgtgt
gcctactgcctcggacttcaaggggctagaattcgatcgacttcttaacccaacagaaggctcgagaaggtatattgctgttgac
agtgagcgtaccctgtagatccgaatttgttagtgaagccacagatgtaacaaattcggatctacagggtctgcctactgcctcg
gacttcaaggggctagaattcgagacttgtttattgcagcttataatggttacaaataaagcaatagcatcacaaatttcacaaata
aagcatttttttcactgcattctagttgtggtttgtccaaactcatcaatgtatcttaacgcggccgagatctccactccctctctgcg
cgctcgctcgctcactgaggccgggcgaccaaaggtcgcccgacgcccgggctttgcccgggcggcctcagtgagcgag
cgagcgcgcagctgcctgcagg
>Seq_77_scAAV-CMV-eGFP-mir-212-5p-mir181b-mir10a (all 22 nt in miR-E backbone),
Guide position
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgcttcagcagataccccgaccatatgaagcagcacgacttcttcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttgacagtgagcgctaagcagtctagagccaaggttagtgaagccacagatgtaaccttggctct
agactgatactgcctactgcctcggacttcaaggggctagaattcgatcgacttcttaacccaacagaaggctcgagaaggta
tattgctgttgacagtgagcgaccaccgacagcaatgaatgtttagtgaagccacagatgtaaacattcattgctgtcggtgggt
gcctactgcctcggacttcaaggggctagaattcgatcgacttcttaacccaacagaaggctcgagaaggtatattgctgttgac
agtgagcgccaaattcggatctacagggtatagtgaagccacagatgtataccctgtagatccgaatttgttgcctactgcctcg
gacttcaaggggctagaattcgagacttgtttattgcagcttataatggttacaaataaagcaatagcatcacaaatttcacaaata
aagcatttttttcactgcattctagttgtggtttgtccaaactcatcaatgtatcttaacgcggccgagatctccactccctctctgcg
cgctcgctcgctcactgaggccgggcgaccaaaggtcgcccgacgcccgggctttgcccgggcggcctcagtgagcgag
cgagcgcgcagctgcctgcagg
>Seq_78_scAAV-CMV-eGFP-mir-181a-mir181b-mir10a (natural pre-miRNAs in miR-
E backbone), Human
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgatcagcagataccccgaccatatgaagcagcacgacttatcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttgtgagttttgaggttgcttcagtgaacattcaacgctgtcggtgagtttggaattaaaatcaaaac
catcgaccgttgattgtaccctatggctaaccatcatctactccactcggacttcaaggggctagaattcgatcgacttcttaacc
caacagaaggctcgagaaggtatattgctgttgcctgtgcagagattattttttaaaaggtcacaatcaacattcattgctgtcggt
gggttgaactgtgtggacaagctcactgaacaatgaatgcaactgtggccccgcttctcggacttcaaggggctagaattcgat
cgacttcttaacccaacagaaggctcgagaaggtatattgctgttggatctgtctgtcttctgtatataccctgtagatccgaatttg
tgtaaggaattttgtggtcacaaattcgtatctaggggaatatgtagttgacataaacactccgctctctcggacttcaaggggct
agaattcgagacttgtttattgcagatataatggttacaaataaagcaatagcatcacaaatttcacaaataaagcatttttttcact
gcattctagttgtggtttgtccaaactcatcaatgtatcttaacgcggccgagatctccactccctctctgcgcgctcgctcgctca
ctgaggccgggcgaccaaaggtcgcccgacgcccgggctttgcccgggcggcctcagtgagcgagcgagcgcgcagct
gcctgcagg
>Seq_79_scAAV-CMV-eGFP-mir-181a-mir181b-mir10a (natural pre-miRNAs in miR-
E backbone), Mouse
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgatcagcagataccccgaccatatgaagcagcacgacttatcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttgggttgcttcagtgaacattcaacgctgtcggtgagtttggaattcaaataaaaaccatcgaccg
ttgattgtaccctatagctaaccctcggacttcaaggggctagaattcgatcgacttcttaacccaacagaaggctcgagaaggt
atattgctgttgaggtcacaatcaacattcattgctgtcggtgggttgaactgtgtagaaaagctcactgaacaatgaatgcaact
gtggccctcggacttcaaggggctagaattcgatcgacttcttaacccaacagaaggctcgagaaggtatattgctgttggacc
tgtctgtcttctgtatataccctgtagatccgaatttgtgtaaggaattttgtggtcacaaattcgtatctaggggaatatgtagttga
cataaacactccgctcactcggacttcaaggggctagaattcgagacttgtttattgcagcttataatggttacaaataaagcaat
agcatcacaaatttcacaaataaagcatttttttcactgcattctagttgtggtttgtccaaactcatcaatgtatcttaacgcggccg
agatctccactccctctctgcgcgctcgctcgctcactgaggccgggcgaccaaaggtcgcccgacgcccgggctttgcccg
ggcggcctcagtgagcgagcgagcgcgcagctgcctgcagg
>Seq_80_scAAV-CMV-eGFP-mir-212-5p-mir181b-mir10a (natural pre-miRNAs in
miR-E backbone), Human
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgatcagcagataccccgaccatatgaagcagcacgacttatcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttgcggggcaccccgcccggacagcgcgccggcaccttggctctagactgcttactgcccggg
ccgccctcagtaacagtctccagtcacggccaccgacgcctggccccgccctcggacttcaaggggctagaattcgatcgac
ttataacc caacagaaggctcgagaaggtatattgctgttgcctgtgcagagattattttttaaaaggtcacaatcaacattcattg
ctgtcggtgggttgaactgtgtggacaagctcactgaacaatgaatgcaactgtggccccgcttctcggacttcaaggggctag
aattcgatcgacttcttaacccaacagaaggctcgagaaggtatattgctgttggatctgtctgtcttctgtatataccctgtagatc
cgaatttgtgtaaggaattttgtggtcacaaattcgtatctaggggaatatgtagttgacataaacactccgctctctcggacttca
aggggctagaattcgagacttgtttattgcagcttataatggttacaaataaagcaatagcatcacaaatttcacaaataaagcatt
tttttcactgcattctagttgtggtttgtccaaactcatcaatgtatcttaacgcggccgagatctccactccctctctgcgcgctcgc
tcgctcactgaggccgggcgaccaaaggtcgcccgacgcccgggcMgcccgggcggcctcagtgagcgagcgagcgc
gcagctgcctgcagg
>Seq_81_scAAV-CMV-eGFP-mir-212-5p-mir181b-mir10a (natural pre-miRNAs in
miR-E backbone), Mouse
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgctcgacccccta
aaatgggcaaacattgcaagcaaacagcaaacacacagccctccctgcctgctgaccttggagctggggcagaggtcagag
acctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggca
gcgtaggcgggcgactcagatcccagccagtggacttagcccctgtttgctcctccgataactggggtgaccttggttaatattc
accagcagcctcccccgttgcccctctggatccactgcttaaatacggacgaggacagggccctgtctcctcagcttcaggca
ccaccactgacctgggacagtgaatccggactctaagaggtaccttaattaagccaccatggtgtccaagggcgaggaactgt
tcaccggcgtggtgcccatcctggtggaactggatggcgacgtgaacggccacaagttcagcgtgtccggcgagggcgaa
ggcgacgccacatatggcaagctgaccctgaagttcatctgcaccaccggcaagctgcccgtgccttggcctaccctcgtga
ccacactgacctacggcgtgcagtgatcagcagataccccgaccatatgaagcagcacgacttatcaagagcgccatgcc
cgagggctacgtgcaggaacggaccatcttctttaaggacgacggcaactacaagaccagggccgaagtgaagttcgagg
gcgacaccctcgtgaaccggatcgagctgaagggcatcgacttcaaagaggacggcaacatcctgggccacaagctggag
tacaactacaacagccacaacgtgtacatcatggccgacaagcagaaaaacggcatcaaagtgaacttcaagatccggcaca
acatcgaggacggctccgtgcagctggccgaccactaccagcagaacacccccatcggagatggccccgtgctgctgccc
gacaaccactacctgagcacacagagcgccctgagcaaggaccccaacgagaagcgggaccacatggtgctgctggaatt
tgtgaccgccgctggcatcaccctgggcatggacgagctgtacaaatgaggcgcgcctcgacttcttaacccaacagaaggc
tcgagaaggtatattgctgttggggcagcgcgccggcaccttggctctagactgcttactgcccgggccgccttcagtaacagt
ctccagtcacggccaccgacgcctggcccctcggacttcaaggggctagaattcgatcgacttcttaacccaacagaaggctc
gagaaggtatattgctgttgaggtcacaatcaacattcattgctgtcggtgggttgaactgtgtagaaaagctcactgaacaatg
aatgcaactgtggccctcggacttcaaggggctagaattcgatcgacttcttaacccaacagaaggctcgagaaggtatattgc
tgttggacctgtctgtcttctgtatataccctgtagatccgaatttgtgtaaggaattttgtggtcacaaattcgtatctaggggaata
tgtagttgacataaacactccgctcactcggacttcaaggggctagaattcgagacttgtttattgcagcttataatggttacaaat
aaagcaatagcatcacaaatttcacaaataaagcatttttttcactgcattctagttgtggtttgtccaaactcatcaatgtatcttaac
gcggccgagatctccactccctctctgcgcgctcgctcgctcactgaggccgggcgaccaaaggtcgcccgacgcccggg
ctttgcccgggcggcctcagtgagcgagcgagcgcgcagctgcctgcagg
>Seq ID No. 83 mir-Ren713, neutral control, miR-E backbone
tcgacttataacccaacagaaggctcgagaaggtatattgctgttgacagtgagcgcaggaattataatgcttatctatagtgaa
gccacagatgtatagataagcattataattcctatgcctactgcctcggacttcaaggggctagaattcga
>Seq ID No. 84, mir-181a stem-loop, miR-E context
tcgacttataacccaacagaaggctcgagaaggtatattgctgtttgagttttgaggttgcttcagtgaacattcaacgctgtcgg
tgagtttggaattaaaatcaaaaccatcgaccgttgattgtaccctatggctaaccatcatctactccatcggacttcaaggggct
agaattcga
>Seq ID No. 85, mir-212 stem-loop, miR-E context
tcgacttcttaacccaacagaaggctcgagaaggtatattgctgttcggggcaccccgcccggacagcgcgccggcaccttg
gctctagactgcttactgcccgggccgccctcagtaacagtctccagtcacggccaccgacgcctggccccgcctcggactt
caaggggctagaattcga

1. Viral vector comprising: a capsid and a packaged nucleic acid, wherein the packaged nucleic acid codes for one or more miRNAs, wherein at least one of the one or more miRNAs comprises the miRNA of Seq ID No. 15, Seq ID No. 17, or Seq ID No. 19.

2. Viral vector according to claim 1, wherein the packaged nucleic acid codes for more than one miRNA, wherein said miRNAs comprise the miRNA of Seq ID No. 15 and the miRNA of Seq ID No. 19 and a miRNA of Seq ID No. 18.

3. Viral vector according to claim 1, wherein the packaged nucleic acid codes for more than one miRNA, wherein said miRNAs comprise the miRNA of Seq ID No. 15 and the miRNA of Seq ID No. 17 and a miRNA of Seq ID No. 18.

4. Viral vector according to claim 1, wherein the packaged nucleic acid codes for more than one miRNA, wherein said miRNAs comprise the miRNA of Seq ID No. 15 and the miRNA of Seq ID No. 17 and the miRNA of Seq ID No. 19.

5. Viral vector according to claim 1, wherein the packaged nucleic acid codes for more than one miRNA, wherein said miRNAs comprise the miRNA of Seq ID No. 15 and the miRNA of Seq ID No. 17.

6. Viral vector according to claim 1, wherein the packaged nucleic acid codes for more than one miRNA, wherein said miRNAs comprise the miRNA of Seq ID No. 15 and the miRNA of Seq ID No. 19.

7. (canceled)

8. Viral vector according to claim 1, wherein the packaged nucleic acid codes for more than one miRNA, wherein said miRNAs comprise the miRNA of Seq ID No. 19 and the miRNA of Seq ID No. 17.

9. Viral vector according to claim 1, wherein the packaged nucleic acid codes for more than one miRNA, wherein said miRNAs comprise the miRNA of Seq ID No. 19 and a miRNA of Seq ID No. 18.

10. Viral vector according to claim 1, wherein the packaged nucleic acid codes for a miRNA having the sequence of Seq ID No. 19, and for a miRNA having the sequence of Seq ID No. 18 and for a miRNA having the sequence of Seq ID No. 17.

11. (canceled)

12. Viral vector according to claim 1, comprising: a capsid and a packaged nucleic acid comprising one or more transgene expression cassettes comprising a transgene that codes
for one or more miRNAs selected from the group consisting of the miRNAs of Seq ID Nos. 15, 17, 18 and 19, and
for an RNA that inhibits the function of one or more miRNAs selected form the group consisting of the miRNAs of Seq ID Nos. 1, 2, 3, 4, 5, 6, 7, 8, 9, 10, 11, 12, 13, 14, 16, 34, 35 and 36.

13. Viral vector according to claim 1, comprising: a capsid and a packaged nucleic acid comprising two or more transgene expression cassettes comprising a transgene,
wherein the first expression cassette comprises a first transgene that codes for one or more miRNAs selected from the group consisting of the miRNAs of Seq ID Nos. 15, 17, 18 and 19, and
wherein the second expression cassette comprises a second transgene that codes for an RNA that inhibits the function of one or more miRNAs selected form the group consisting of miRNAs of Seq ID No 1, 2, 3, 4, 5, 6, 7, 8, 9, 10, 11, 12, 13, 14, 16, 34, 35 and 36.

14.-15. (canceled)

16. Viral vector according to claim 12, wherein the transgene expression cassettes comprise a promotor, a transgene and a polyadenylation signal, wherein promotors or the polyadenylation signals are positioned opposed to each other.

17. Viral vector according to claim 1, wherein the vector is a recombinant AAV vector.

18. Viral vector according to claim 1, wherein the vector is a recombinant AAV vector having the AAV-2 serotype.

19. Viral vector according to claim 1, wherein the capsid comprises a first protein that comprises the sequence of Seq ID No. 29 or 30.

20. Viral vector according to claim 1, wherein the capsid comprises a first protein that is 80% identical to a second protein having the sequence of Seq ID No. 82, whereas one or more gaps in the alignment between the first protein and the second are allowed.

21. Viral vector according to claim 1, wherein the capsid comprises a first protein that is 95% identical to a second protein of Seq ID No. 82, whereas a gap in the alignment between the first protein and the second protein is counted as a mismatch.

22. Viral vector according to claim 1, wherein the vector is a recombinant AAV vector having the AAV5 or the AAV6.2 serotype, and wherein the capsid of the recombinant AAV6.2 vector preferably comprises a capsid protein having the sequence of Seq ID No. 82.

23.-25. (canceled)

26. Method of treating a disease selected from the group consisting of PF-ILD, IPF, connective tissue disease (CTD)-associated ILD, rheumatoid arthritis ILD, chronic fibrosing hypersensitivity pneumonitis (HP), idiopathic non-specific interstitial pneumonia (iNSIP), unclassifiable idiopathic interstitial pneumonia (IIP), environmental/occupational lung disease, systemic sclerosis ILD, sarcoidosis, and fibrosarcoma, the method comprising administering to a patient in need thereof a therapeutically active amount of viral vector according to claim 1.

27. (canceled)

28. AAV vector comprising a vector genome that codes for one or more miRNAs selected from the group comprising the miRNA of Seq ID No. 15, the miRNA of Seq ID No. 17, and the miRNA of Seq ID No. 19.

29. AAV vector according to claim 28, wherein said vector genome codes for a miRNA having the sequence of Seq ID No. 15 and for a miRNA having the sequence of Seq ID No. 17 and optionally for a miRNA having the sequence of Seq ID No. 19.

30. AAV vector according to claim 28, wherein said vector genome further codes for an RNA that inhibits the function of one or more miRNAs selected form the group consisting of the miRNAs of Seq ID Nos. 1, 2, 3, 4, 5, 6, 7, 8, 9, 10, 11, 12, 13, 14, 16, 34, 35 and 36.

31. (canceled)

32. A miRNA mimetic for use in a method of prevention and/or treatment of a fibroproliferative disorder, wherein miRNA comprises the sequence of Seq ID No. 15.

33. A miRNA mimetic of miRNA 212-5p for use in a method according to claim 32, wherein the miRNA mimetic is an oligomer of nucleotides that consist of the sequence of Seq ID No. 15, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 15;
the oligomer optionally comprises nucleotide analogues that show the base-pairing behavior at the corresponding position (AU and GC) as determined by the sequence of Seq ID No. 15;
the oligomer is optionally lipid conjugated to facilitate drug delivery.

34. A miRNA mimetic for use in a method according to claim 32, wherein said prevention and/or treatment further comprises the administration of a mimetic of a miRNA having the sequence of Seq ID No. 19 or a mimetic of a miRNA having the sequence of Seq ID No. 18, or a mimetic of a miRNA having the sequence of Seq ID No. 17.

35. A miRNA mimetic for use in a method according to claim 32, wherein said prevention and/or treatment further comprises the administration of a mimetic of a miRNA having the sequence of Seq ID No. 17.

36. A miRNA mimetic for use in a method according to claim 32, wherein said prevention and/or treatment further comprises the administration of a miRNA mimetic of miRNA 181a-5p, and wherein the miRNA mimetic is an oligomer of nucleotides that consists of the sequence of Seq ID No. 17, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 17;
the oligomer optionally comprises nucleotide analogues that show the base-pairing behavior at the corresponding position (AU and GC) as determined by the sequence of Seq ID No. 17;
the oligomer is optionally lipid conjugated to facilitate drug delivery.

37. A miRNA mimetic for use in a method according to claim 32, wherein said prevention and/or treatment further comprises the administration of a mimetic of a miRNA having the sequence of Seq ID No. 19.

38. A miRNA mimetic for use in a method according to claim 32, wherein said prevention and/or treatment further comprises the administration of a miRNA mimetic of miRNA 181b-5p, and wherein the miRNA mimetic is an oligomer of nucleotides that consists of the sequence of Seq ID No. 19, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 19;
the oligomer optionally comprises nucleotide analogues that show the base-pairing behavior at the corresponding position (AU and GC) as determined by the sequence of Seq ID No. 19;
the oligomer is optionally lipid conjugated to facilitate drug delivery.

39. A miRNA mimetic for use in a method according to claim 32, wherein said prevention and/or treatment further comprises the administration of a mimetic of a miRNA having the sequence of Seq ID No. 17 and a mimetic of a miRNA having the sequence of Seq ID No. 19.

40. A miRNA mimetic for use in a method according to claim 32, wherein said prevention and/or treatment further comprises the administration of a mimetic of a miRNA having the sequence of Seq ID No. 18 and a mimetic of a miRNA having the sequence of Seq ID No. 19.

41. A miRNA mimetic according to claim 32, wherein said prevention and/or treatment further comprises the administration of a mimetic of a miRNA having the sequence of Seq ID No. 17 and a mimetic of a miRNA having the sequence of Seq ID No. 18.

42. A miRNA mimetic for use in a method according to claim 32, wherein the fibroproliferative disorder is IPF or PF-ILD.

43. (canceled)

44. Pharmaceutical composition comprising (i) a miRNA mimetic of a miRNA having the sequence of Seq ID No. 15, or (ii) a miRNA mimetic of a miRNA having the sequence of Seq ID No. 17, or (iii) a miRNA mimetic of a miRNA having the sequence of Seq ID No. 18, or (iv) a miRNA mimetic of a miRNA having the sequence of Seq ID No. 19, and a pharmaceutical-acceptable carrier or diluent.

45. Pharmaceutical composition according to claim 44, comprising both a miRNA mimetic of a miRNA having the sequence of Seq ID No. 15 and either a miRNA mimetic of a miRNA having the sequence of Seq ID No. 17 or a miRNA mimetic of a miRNA having the sequence of Seq ID No. 19, and said pharmaceutical-acceptable carrier or diluent.

46. Pharmaceutical composition according to claim 44 comprising
(a) said miRNA mimetic of a miRNA having the sequence of Seq ID No. 15, wherein said miRNA mimetic is a miRNA mimetic of miRNA 212-5p, wherein the miRNA mimetic is an oligomer of nucleotides that consists of the sequence of Seq ID No. 15, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 15;
the oligomer optionally comprises nucleotide analogues that show the base-pairing behavior at the corresponding position (AU and GC) as determined by the sequence of Seq ID No. 15;
the oligomer is optionally lipid conjugated to facilitate drug delivery; and
(b) said miRNA mimetic of a miRNA having the sequence of Seq ID No. 17, wherein said miRNA mimetic is a miRNA mimetic of miRNA 181a-5p, wherein the miRNA mimetic is an oligomer of nucleotides that consists of the sequence of Seq ID No. 17, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 17;
the oligomer optionally comprises nucleotide analogues that show the base-pairing behavior at the corresponding position (AU and GC) as determined by the sequence of Seq ID No. 17,
the oligomer is optionally lipid conjugated to facilitate drug delivery; and
(c) said pharmaceutical-acceptable carrier or diluent.

47. Pharmaceutical composition according to claim 44, comprising
(a) said miRNA mimetic of a miRNA having the sequence of Seq ID No. 15, wherein said miRNA mimetic is a miRNA mimetic of miRNA 212-5p, wherein the miRNA mimetic is an oligomer of nucleotides that consist of the sequence of Seq ID No. 15, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 15;
the oligomer optionally comprises nucleotide analogues that show the base-pairing behavior at the corresponding position (AU and GC) as determined by the sequence of Seq ID No. 15; and
the oligomer is optionally lipid conjugated to facilitate drug delivery, and
(b) said miRNA mimetic of a miRNA having the sequence of Seq ID No. 19, wherein said miRNA mimetic is a miRNA mimetic of miRNA 181b-5p, wherein the miRNA mimetic is an oligomer of nucleotides that consist of the sequence of Seq ID No. 19, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 19;
the oligomer optionally comprises nucleotide analogues that show the base-pairing behavior at the corresponding position (AU and GC) as determined by the sequence of Seq ID No. 19,
the oligomer is optionally lipid conjugated to facilitate drug delivery; and
(c) said pharmaceutical-acceptable carrier or diluent.

48. Method of treating a disease selected from the group consisting of PF-ILD, IPF, connective tissue disease (CTD)-associated ILD, rheumatoid arthritis ILD, chronic fibrosing hypersensitivity pneumonitis (HP), idiopathic non-specific interstitial pneumonia (iNSIP), unclassifiable idiopathic interstitial pneumonia (IIP), environmental/occupational lung disease, systemic sclerosis ILD, sarcoidosis, and fibrosarcoma, the method comprising administering to a patient in need thereof a therapeutically active amount of a pharmaceutical composition according to claim 44.

49. (canceled)
